SEQUENCE LISTING (2 sequences)

SEQ ID NO: 1 - DNA, 20 bp, Artificial Sequence
  Description of sequence  primer FSequence (see p. 44)
ctttcatcaactcgcgatgc

SEQ ID NO: 2 - DNA, 19 bp, Artificial Sequence
  Description of sequence  primer RSequence (see p. 44)
gctcatggtgccaatggag

(19) United States
(12) Patent Application Publication
(10) Pub. No.: US 2011/0151020 A1
(43) Pub. Date: Jun. 23, 2011

(54) ACTIVE SUBSTANCE COMBINATION WITH GEMCITABINE FOR THE TREATMENT OF EPITHELIAL CANCER

(76) Inventors: CHRISTOPHER HEESCHEN, MADRID (ES); MARIA THERESA MÜLLER, MUNCHEN (DE); PATRICK CHRISTIAN HERMANN, MADRID (ES); STEPHAN HUBER, MUNCHEN (DE)

(21) Appl. No.: 12/880,972

(22) Filed: Sep. 13, 2010

Related U.S. Application Data
(63) Continuation-in-part of application No. PCT/EP2009/001795, filed on Mar. 12, 2009.

(30) Foreign Application Priority Data
Mar. 12, 2008 (EP) 08004631.1

(51) Int. Cl. A61K 33/36; A61K 31/7068; A61P 35/00; A61K 38/00; A61K 31/7076

(52) U.S. Cl. 424/623; 514/49; 514/19.3; 514/47

(57) ABSTRACT

The present invention refers to active substance combinations comprising of a nucleoside analog or antimetabolic agent like Gemcitabine, and either a Nodal/Activin inhibitor or a SHH-Inhibitor and an mTOR-inhibitor, medicaments comprising the same and the use of the active substance combinations in the treatment of cancer, especially of epithelial cancer.

### RELATED APPLICATIONS

This application is a continuation-in-part of international Application No. PCT/EP2009/001795, filed Mar. 12, 2009, and published as WO 2009/112266 A1 on Sep. 17, 2009, which claims priority from European application no. 08004631.1, the disclosures of which are incorporated by reference.

### FIELD OF THE INVENTION

The present invention refers to active substance combinations comprising of a nucleoside analog or antimetabolic agent like Gemcitabine, and either a Nodal/Activin inhibitor or a SHH-Inhibitor and an mTOR-inhibitor, medicaments comprising the same and the use of the active substance combinations in the treatment of cancer, especially of epithelial cancer.

### BACKGROUND OF THE INVENTION

Epithelial cancers are among the most frequent causes of death. Especially pancreatic carcinomas are characterized by early metastatic spread and a pronounced resistance to chemotherapy and radiation. Despite extensive research activities in the field of tumour biology, there has hardly been any substantial progress within the past decades regarding therapeutic success. The introduction of the chemotherapeutic agent Gemcitabine improved clinical response by reducing pain and loss of weight. As the median 5-year survival rate (1-4%) and the median survival time (5 months) are very low, the prognosis of patients with pancreatic cancer has remained poor.

Within the last years, it has been shown that stem cells play a decisive role in the development and progression of cancer, and that distinct populations of cells with stem cell properties may be essential for the development and perpetuation of various human cancers, including pancreatic cancer, colon cancer, lung cancer, breast cancer and brain tumour. According to the current consensus definition, a tumour cell that has the ability to self-renew, is exclusively tumorigenic, and is capable of producing the heterogeneous lineages of cancer cells that comprise the tumour fulfils the criteria of a cancer stem cell (CSC).

Only recently the role of CSC in pancreatic cancer and in metastasis has also been defined (Ho, M. M., Ng, A. V., Lam, S., and Hung, J. Y. 2007). Side population in human lung cancer cell lines and tumours is enriched with stem-like cancer cells. Cancer Res 67:4827-4833 and O'Brien, C. A., Pollett, A., Gallinger, S., and Dick, J. E. 2007. A human colon cancer cell capable of initiating tumour growth in immunodeficient mice. Nature 445:106-110). It was further demonstrated that the CSC population contained in several tumour entities is responsible for resistance to therapy (Ho, et al. 2007, see above; Ma, S., Lee, T. K., Zheng, B. J., Chan, K. W., and Guan, X. Y. 2007. CD133⁺ HCC cancer stem cells confer chemoresistance by preferential expression of the Akt/PKB survival pathway. Oncogene; and Phillips, T. M., McBride, W. H., and Pajonk, F. 2006. The response of CD24(−/low)/CD44+ breast cancer-initiating cells to radiation. J Natl Cancer Inst 98:1777-1785). Accordingly, it seems to be of the utmost importance to discover new treatment modalities leading to the elimination of CSC in order to eventually develop protocols for more successful treatment of cancer, especially of pancreatic cancer.

Most desirably, these novel therapies—including new active substance combinations—would provide a more effective treatment modality for cancer patients and therefore would increase the 5-year survival rate of the patients.

Desirably these novel therapies—including new active substance combinations—would allow the reduction of chemotherapeutic agents to be used in that therapy in the light of their well-known side effects.

It would also be desirable if any new active substance combinations would show an additive effect of the active substances, thus again allowing the reduction of any chemotherapeutic agent to be used therein.

Unfortunately, conventional therapy using chemotherapy or radiation seems to have little to no effect on cancer stem cells. As will also be demonstrated below treatment with the anti-metabolite Gemcitabine, the first-line chemotherapeutic agent for the treatment of pancreatic cancer had no detectable effect on CD133+ cancer stem cells. Cell cycle analyses even showed that CSC immediately entered a state of cell divisions and therefore started the repopulation of the tumour immediately after the withdrawal of standard therapy (Hermann, P. C., Huber, S. L., Herrler, T., A., A., Ellwart, J. W., Guba, M., Bruns, C., and Heeschen, C. 2007. Distinct Populations of Cancer Stem Cells Determine Tumour Growth and Metastatic Activity in Human Pancreatic Cancer. Cell Stem Cell 1:313-323). Thus, CD133+ tumour cells are highly enriched for the tumorigenic cancer stem cell fraction following Gemcitabine therapy. Although—as shown below—in vivo therapy with Gemcitabine resulted in local tumour growth in an orthotopic mouse model of xenotransplanted human pancreatic cancer, a significant enrichment of CD133+ cells in the residual tumour tissue could be demonstrated. Consequently, the withdrawal of Gemcitabine will soon result in relapse, in most cases with an even more aggressive phenotype.

The accumulating evidence that standard therapy in many malignancies does not affect the suspected root of the disease, namely the cancer stem cells, emphasizes the urgent need for either developing targeted therapies against these cells or modifying current treatment modalities in order to be able to eliminate these cells (‘chemo-sensitizing’). Considering that according to the CSC hypothesis only CSC are able to reproduce the heterogeneous lineages of cancer cells, attacking a tumour's CSC population seems to be the most promising approach for new developments. If a tumour is depleted of CSC, it loses its exclusive source for progression and metastasis, and should eventually degrade due to the limited life span of more differentiated tumour cells. As shown previously, a single CSC is able to reproduce an entire tumour (Zucchi, I., et al. 2007. The properties of a mammary gland cancer stem cell. PNAS 104: 10476-10481) Therefore, any CSC remaining after supposedly successful therapy would inevitably lead to tumour relapse. This undoubtedly shows that it is of utmost importance to develop novel therapies, which primarily target CSC and lead to their complete elimination.

This object was achieved by the active substance combinations according to the invention as demonstrated by in-vitro—and in-vivo—experiments shown below.

Thus, the present invention relates to an active substance combination comprising
(A) at least one nucleoside analog and/or a further anti-metabolitic agent, preferably capable to interrupt or interfere with DNA replication or synthesis, and
(B) either

(B1) at least one Nodal/Activin inhibitor,

or

(B2) an active substance combination of

(B2a) at least one SHH inhibitor and

(B2b) at least one mTOR inhibitor.

According to this invention a “nucleoside analog” is defined as a synthetic molecule that resembles a naturally occurring nucleoside, but that lacks a bond site needed to link it to an adjacent nucleotide. In a more narrow definition the “nucleoside analog” is at the same time an antimetabolite and preferably as an antineoplastic agent. Anti-metabolites may resemble purine or pyrimidine (e.g., of of a naturally occurring nucleoside) but prevent these purines or pyrimidines from becoming incorporated in to DNA during the “S” phase (of the cell cycle). This interference usually terminates DNA synthesis and replication and may lead to apoptosis of the cell. A “nucleoside analog” used according to the present invention is therefore preferably an analog of a naturally occurring nucleoside, more preferably a (structurally similar) nucleoside, wherein the nucleoside analog as defined above is different enough to ensure that the resultant DNA or RNA is non-functional, when incorporated into DNA or RNA during DNA or RNA synthesis. Typically, nucleoside analogs comprising nucleobase modifications confer, among other things, different base pairing and base stacking proprieties, while nucleoside analogs comprising phosphate-sugar backbone modifications typically affect the properties of the chain. Preferably, a nucleoside analog as defined above inhibits or terminates DNA replication (or RNA synthesis) in normal human DNA replication, and optionally DNA replication by reverse transcriptase. Most preferably, a nucleoside analog as defined above inhibits or terminates DNA replication (or RNA synthesis) in normal human DNA replication, wherein DNA replication by reverse transcriptase is not or only in part affected. Nucleoside analogs may be identified using a simple proliferation test, e.g., using human cells such as HeLa cells and a nucleoside analog as defined above, wherein a significant reduction of cells typically indicates a termination of DNA synthesis and apoptosis of said cells. Typically, such nucleoside analogs are preferred, which exhibit at least 50% of the activity of gemcitabine, more preferably at least 60% of the activity of gemcitabine, even more preferably 70%, 80% or 90% of the activity of gemcitabine, most preferably 95%, 96%, 97%, 98%, 99% or even 100% of the activity of gemcitabine. The activity of gemcitabine may be defined as its capability to inhibit or terminate DNA replication (or RNA synthesis) in normal human DNA replication.

A nucleoside analog as defined above may be selected from a naturally occurring nucleoside, wherein one or more naturally occurring functional moieties or groups thereof, such as hydrogen groups, hydroxy groups, methyl groups, amino groups, etc., have been substituted by non-naturally occurring moieties. Such substitutions may be carried out either in the ribose (sugar), in the phosphate backbone or in the naturally occurring nucleobase. Preferably, non-naturally occurring moieties may include chemical substituents or groups, e.g., chemical moieties, such as —CN, —NC, methyl groups, amino or imino groups, atoms such as halogenes, including as fluorine, chlorine, bromine or iodine, etc.

A nucleoside analog may furthermore comprise a (structurally similar) nucleoside, wherein the nucleoside analogue resembles the structure of the naturally occurring nucleoside.

Nucleoside analogs used according to the present invention may preferably include, inter alia:
pyrimidine analogs, including, gemcitabine, 5-Fluoruracil, Capecitabine, Cytarabine (Ara-C), Floxuridine, etc.;
purine analogs, including Azathioprine, 6-Mercaptopurine, 6-Thioguanine, Fludarabine, Pentostatin, etc.;
Purine antimetabolites, including Fludarabine, etc.

The preferred example of a nucleoside analog for this invention is Gemcitabine (IUPAC name: 4-amino-1-[3,3-difluoro-4-hydroxy-5-(hydroxymethyl)tetrahydrofuran-2-yl]-1H-pyrimidin-2-one). Gemcitabine is a pyrimidine analog, marketed as Gemzar™, in which the hydrogen atoms on the 2′ carbons of deoxycytidine are replaced by fluorine atoms.

Fluorouracil (5-FU or f5U) (IUPAC name: 5-fluoro-1H-pyrimidine-2,4-dione) is a pyrimidine analog, which is used as a drug in the treatment of cancer. It principally acts as a thymidylate synthase inhibitor. Interrupting the action of this enzyme blocks synthesis of the pyrimidine thymidine, which is a nucleotide required for DNA replication. Thymidylate synthase methylates deoxyuridine monophoshate (dUMP) into thymidine monophosphate (dTMP).

Capecitabine (IUPAC name: pentyl[1-(3,4-dihydroxy-5-methyl-tetrahydrofuran-2-yl)-5-fluoro-2-oxo-1H-pyrimidin-4-yl]aminomethanoate) is a pyrimidine analog, which acts as a prodrug, that is enzymatically converted to 5-fluorouracil in the tumor. There, it inhibits DNA synthesis and slows growth of tumor tissue. The activation of capecitabine follows a pathway with three enzymatic steps and two intermediary metabolites, 5′-deoxy-5-fluorocytidine (5′-DFCR) and 5′-deoxy-5-fluorouridine (5′-DFUR), to form 5-fluorouracil. Capecitabine is marketed under the trade name Xeloda.

Cytarabine, or cytosine arabinoside, is an antimetabolic agent with the chemical name of 1-arabinofuranosylcytosine (IUPAC name: 4-amino-1-[(2R,3S,4R,5R)-3,4-dihydroxy-5-(hydroxymethyl)oxolan-2-yl]pyrimidin-2-one, also abbreviated Ara-C). Its mode of action is due to its rapid conversion into cytosine arabinoside triphosphate, which damages DNA when the cell cycle holds in the S Phase (synthesis of DNA) or interrupts DNA synthesis. Rapidly dividing cells, which require DNA replication for mitoses, are therefore most affected. Cytosine arabinoside also inhibits both DNA and RNA polymerases and nucleotide reductase enzymes needed for DNA synthesis.

Floxuridine (FUDR) (IUPAC name: 5-Fluoro-1-[4-hydroxy-5-(hydroxymethyl)-tetrahydrofuran-2-yl]-1H-pyrimidine-2,4-dione) is a pyrimidine analog, which is used as a drug in the treatment of cancer.

Azathioprine (IUPAC name: 6-[(1-methyl-4-nitro-1H-imidazol-5-yl)sulfanyl]-7H-purine) is a purine analog and a purine synthesis inhibitor, inhibiting the proliferation of cells.

Mercaptopurine (also called 6-Mercaptopurine, 6-MP or its brand name Purinethol) (IUPAC name: 3,7-dihydropurine-6-thione) is converted to the corresponding ribonucleotide. 6-MP ribonucleotide inhibits purine nucleotide synthesis and metabolism. This alters the synthesis and function of RNA and DNA. Mercaptopurine interferes with nucleotide interconversion and glycoprotein synthesis.

Thioguanine (IUPAC name: 2-amino-7H-purine-6-thiol) is a purine/guanine analog and is transformed inside the cell into 6-thioguanilyic acid (TGMP). TGMP interferes by pseudofeedback interference with purine biosynthesis with the synthesis of guanine nucleotides. It is further incorporated of thioguanine nucleotides into both RNA and DNA but the end-result is inducing cell cycle arrest and apoptosis.

Fludarabine (IUPAC name: [(2R,3R,4S,5R)-5-(6-amino-2-fluoro-purin-9-yl)-3,4-dihydroxy-oxolan-2-yl]methoxyphosphonic acid) is both a purine analog and a purine antimetabolite. It inhibits DNA synthesis by interfering with ribonucleotide reductase and DNA polymerase. It is active against both dividing and resting cells.

Pentostatin (deoxycoformycin) (IUPAC name: (8R)-3-(2-deoxy-D-erythro-pentofuranosyl)-3,4,7,8-tetrahydroimidazo[4,5-d][1,3]diazepin-8-ol) is a purine analog and mimics the nucleoside adenosine and thus inhibits the enzyme adenosine deaminase, thereby interfering with the DNA processing and synthesis

Additionally or alternatively to a nucleoside analog as defined above, the active substance combination(s) as defined herein may contain as a further component (A), a further a further anti-metabolitic agent, i.e., a further compound, which is capable to stop or interrupt DNA synthesis when the cell cycle holds in the S Phase (synthesis of DNA). Such a compound may be selected from chemotherapeutic agents, including, without being limited thereto:
Anthracyclines, including Daunorubicin, Doxorubicin (Adriamycin), Epirubicin, Idarubicin, etc.
Folate analogs, including methothrexate, etc.;
Ribonucleotide reductase inhibitors, including hydroxyurea, etc.

Such further anti-metabolitic agents are preferred, which exhibit at least 50% of the activity of gemcitabine, more preferably at least 60% of the activity of gemcitabine, even more preferably 70%, 80% or 90% of the activity of gemcitabine, most preferably 95%, 96%, 97%, 98%, 99% or even 100% of the activity of gemcitabine. The activity of gemcitabine may be defined as its capability to inhibit or terminate DNA replication (or RNA synthesis) in normal human DNA replication.

Many regulatory functions in embryonic as well as adult stem cells are mediated by the Sonic Hedgehog (SHH) pathway. Dysregulations in this pathway are usually lethal in early embryonic stages. Mutations in the SHH pathway have been identified in a large variety of malignant tumours. Hedgehog is the extracellular component of the pathway and activates intracellular signals after binding to its specific receptor “Patched” (Ptch), a protein located on the cellular wall. After binding of Hedgehog to Patched a protein called “Smoothened” (SMO) becomes activated and thus induces transcription of target genes of the Hedgehog pathway. In the absence of Hedgehog the activity of Smoothened is suppressed by Patched and thus the target genes of the Hedgehog pathway are not expressed. A scheme illustrating the Sonic Hedgehog Pathway scheme can be found in FIG. 41.

Accordingly “SHH-Inhibitors” are defined as compounds targeting components of the hedge-hog signalling pathway, thus inhibiting its activity. Examples of SHH-inhibitors include Cyclopamine, Cyclopamine-KAAD, Jervine, SANT-1 and CUR 61414, all of them antagonists binding to SMO; Forskolin, an cAMP enhancer; as well as arsenic Trioxide(ATO) binding Gli, or the hedgehog antagonist CUR-0199691.

Cyclopamine (11-deoxojervine) is a natural occurring steroidal jerveratrum alkaloid influencing the balance between active and inactive SMO and is freely available through chemical suppliers.

Cyclopamine-KAAD (3-Keto-N-(aminoethyl-aminocaproyl-dihydrocinnamoyl) Cyclopamine) is a variant of Cyclopamine, also influencing the balance between active and inactive SMO and is freely available through chemical suppliers.

Jervine is another natural occurring steroidal alkaloid from veratrum against a SMO antagonist and is freely available through chemical suppliers.

CUR 61414 is also a small molecule SHH-Inhibitor developed by CURIS Inc., USA.

SANT-1 (N-[(3,5-dimethyl-1-phenyl-1H-pyrazol-4-yl)methylene]-4-(phenylmethyl)-1-piperazinamine) is a potent inhibitor of SHH being an antagonist of SMO activity, and is freely available through chemical suppliers.

Forskolin is a natural occurring labdane diterpene commonly used to raise the level of cAMP and is also freely available from chemical suppliers.

Arsenic Trioxide (ATO) is a well-know derivative of arsenic available through medical suppliers.

mTOR (mammalian target of Rapamycin) is a serine/threonine kinase from the superfamily of the Phosphatidylinositole-3-kinase (PI-3K) like kinases. It is involved in signalling of proliferatory impulses and the regulation of cellular homeostasis. mTOR is the target gene in a complex pathway, which is influenced by growth factors as well as intracellular energy levels and local supplies os oxygen. Inhibition of mTOR leads to downregulation of translation of several target genes of mTOR. Several mTOR inhibitors have already been approved as immunosuppressants following organ transplantation.

Accordingly “mTOR-Inhibitors” are defined as compounds binding and inhibiting the serine/threonine kinase mTOR. Examples of well-known mTOR-inhibitors include Rapamycin, Temsirolimus (CCI-779), Everolimus (RAD 001), Deforolimus (AP 23573) and TAFA 93, most of them easily available through commercial suppliers. A scheme illustrating the mTOR pathway can be found in FIG. 42.

In the context of the present invention, a “Nodal/Activin-Inhibitor” (also termed “Nodal-Inhibitor”) is preferably understood as an inhibitor inhibiting Nodal and/or Activin signalling. Nodal as well as Activin are both members of the Transforming Growth Factor (TGF)-β superfamily and play an essential role in embryonic development, particularly for maintaining the pluripotency of embryonic stem cells. Nodal and Activin signaling is both mediated through the receptors Activin receptor Like Kinase (ALK)-4 and -7. Nodal activates the Smad 2/3 signalling pathway via ALK4 and ALK7. While this pathway is pro-apoptotic in adult cells, Nodal signalling transduction in an embryonic context results in elevated proliferation and invasiveness with subsequent ectopic cell and organ growth. Nodal was also identified as key molecule for tumorigenicity and especially invasiveness of malignant melanoma (Topczewska, J. M., et al. 2006. Embryonic and tumorigenic pathways converge via Nodal signalling: role in melanoma aggressiveness. Nat Med. 12;8:925-932). Specifically, Nodal signaling has been found to be active not only in embryonic stem cells but also involved in the maintenance of an aggressive phenotype in melanoma and breast cancer cells. Furthermore, inhibition of Nodal signaling using the specific antagonist Lefty has been shown to reduce tumorigenicity in melanoma cell lines, underlining its potential to target stemness in malignancies. An important co-receptor for Nodal is Cripto-1. Based on these findings the present inventors surprisingly found that the influence of Nodal and Activin on the tumorigenic cell compartment of pancreatic cancer and its potential as a therapeutic target in combination therapy is of utmost importance. A scheme illustrating the Nodal signalling pathway can be found in FIG. 43.

Accordingly “Nodal/Activin-Inhibitors” (also termed “Nodal-Inhibitors”) are defined as compounds inhibiting the Nodal pathway and/or preferably the Activin pathway, either by inhibiting the ALK receptors, especially ALK 4 or 7, or by being Nodal antagonists or activin antagonists. Such “Nodal/Activin-Inhibitors” include both inhibitors of Nodal and/or inhibitors of Activin, as both inhibitors are effective in the inventive context. Examples of such “Nodal/Activin-Inhibitors” include SB431542 (a specific inhibitor of the receptors for Nodal and Activin) the Coco-Protein, the Nicalin-Protein, the Nomo-Protein, Folistatin or Lefty.

SB431542 is a known inhibitor of Nodal and of Activin acting on the TGFb family receptors ALK4, 5 and 7 and is available through commercial sources.

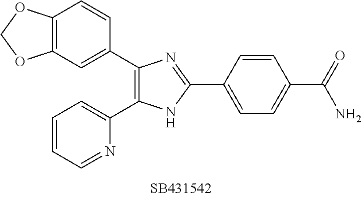

The Coco-Protein (also described as 51-B6) is described inter alia by Bell et al. (2003) Development 130,1381-1389. It is related to Accession No. NP 001092196.

The Nicalin-Protein is described/mentioned inter alia by Haffner et al. (2004) EMBO 15; 3041-3050 and Hafner et al. (2007), J. Biol. Chem. 282(14); 10632-8. It is related to Accession No NP 064555

The Nomo-Protein/s (also known as pM5) is described/mentioned inter alia by Haffner et al. (2004) EMBO 15; 3041-3050 and Hafner et al. (2007), J. Biol. Chem. 282(14); 10632-8. It/they are related to Accession Nos. AAH65535, NP 001004067, NP 001004060, and NP 775885.

Follistatin is a single chain autocrine glycoprotein found to be ubiquitous within the body of nearly all higher animals that is the product of a single gene. It was initially isolated from follicular fluid and was identified as a protein fraction that inhibited Follicle-stimulating hormone (FSH) secretion from the anterior pituitary, and so was known as FSH-suppressing protein (FSP). Since then its primary function has been determined to be the binding and bioneutralization agent of members of the TGF-beta superfamily, with primary focus on Activin, which enhances secretion of FSH in the anterior pituitary.

Lefty proteins, particularly Lefty1, are extracellular antagonists of Nodal. Lefty proteins are involved in embryogenesis and left-right patterning, e.g., assigning differences between the left and right sides, including heart and lung positioning. They specifically regulate the degree of left-right asymmetry during vertebrate development by controlling the spatiotemporal influence of the Nodal protein. Mutations in these genes cause incorrect positioning of these organs (e.g., situs invertis). Known Lefty proteins include Lefty 1 and 2. Lefty1 in the ventral midline prevents the Cerebrus (paracrine factor or “Caronte”) signal from passing to the right side of the embryo. The role of Lefty1 is to restrict the expression of Lefty2 and Nodal to the left side, and to prevent Lefty2 or Nodal to encode a signal for ‘leftness.’ Lefty2 serves as a feedback inhibitor to restrict the range of nodal signaling during establishment of the left-right axis.

In one preferred embodiment of the active substance combination according to the invention the nucleoside analog is Gemcitabine or is selected from pyrimidine analogs, including, gemcitabine, 5-Fluoruracil, Capecitabine, Cytarabine (Ara-C), or Floxuridine; or from purine analogs, including Azathioprine, 6-Mercaptopurine, 6-Thioguanine, Fludarabine, or Pentostatin; or from Purine antimetabolites, including Fludarabine.

In one preferred embodiment of the active substance combination contains as a further component (A), a further anti-metabolitic agent selected from Anthracyclines, including Daunorubicin, Doxorubicin (Adriamycin), Epirubicin, or Idarubicin; or from Folate analogs, including methothrexate; or from Ribonucleotide reductase inhibitors, including hydroxyurea.

According to another preferred embodiment, the active substance combination may contain a combination of these nucleoside analogs and/or of the further anti-metabolitic agents, typically at least one of these nucleoside analogs and/or of these further anti-metabolitic agents, e.g., at least two, three, or even more of these nucleoside analogs and/or of these further anti-metabolitic agents.

In another preferred embodiment of the active substance combination according to the invention the Nodal inhibitor or Nodal/Activin-Inhibitor is selected from SB431542, Coco-Protein, Nicalin-Protein or Nomo-Protein.

In a further preferred embodiment of the active substance combination according to the invention the SHH-Inhibitor is selected from Cyclopamine, Cyclopamine-KAAD, Jervine, CUR 61414, Forskolin, SANT-1, Arsenic Trioxide (ATO), or CUR-0199691.

In yet one further preferred embodiment of the active substance combination according to the invention the mTOR-Inhibitor is selected from Rapamycin, Temsirolimus (CCI-779), Everolimus (RAD 001), Deforolimus (AP 23573) or TAFA 93.

Any of the above preferred embodiments may be combined as suitable.

Another highly preferred embodiment of the invention refers to an Active Substance Combination according to the invention comprising

(A) Gemcitabine
and

(B) either
(B1) at least one Nodal/Activin-Inhibitor, preferably selected from SB431542, Coco-Protein, Nicalin-Protein, Nomo-Protein, Folistatin or Lefty, more preferably being B431542;
   or
(B2) an active substance combination of
   (B2a) at least one SHH inhibitor, preferably selected from Cyclopamine, Cyclopamine-KAAD, Jervine, CUR 61414, Forskolin, SANT-1, Arsenic Trioxide, or CUR-0199691, more preferably being Cyclopamine;
and
   (B2b) at least one mTOR inhibitor, preferably selected from Rapamycin, Temsirolimus (CCI-779), Everolimus (RAD 001), Deforolimus (AP 23573) or TAFA 93, more preferably being Rapamycin.

In the above defined exemplary Active Substance Combination, the nucleoside analog gemcitabine may be replaced by any of the nucleoside analogs as defined herein or a combination of these nucleoside analogs as defined above. Alternatively, in the above-defined exemplary Active Substance Combination, the nucleoside analog gemcitabine may be replaced by any of the further anti-metabolitic agents as defined above or a combination of these further anti-metabolitic agents as defined above.

One other preferred embodiment of the invention refers to an Active Substance Combination (1) according to the invention comprising
(A) Gemcitabine
   and
(B1) at least one Nodal/Activin-Inhibitor, preferably selected from SB431542, Coco-Protein, Nicalin-Protein, Nomo-Protein, Folistatin or Lefty, more preferably being SB431542.
   Preferably this Active Substance Combination (1) is selected from a combination of
Gemcitabine and SB431542,
Gemcitabine and Coco-Protein,
Gemcitabine and Nicalin-Protein,
Gemcitabine and Nomo-Protein,
Gemcitabine and Folistatin, or
Gemcitabine and Lefty.

In one embodiment the Active Substance Combinations (1) listed above are consisting of the active substances listed in each combination.

One embodiment of the invention refers to an Active Substance Combination (1) according to the invention consisting of
(A) Gemcitabine
   and
(B1) at least one Nodal/Activin-Inhibitor, preferably selected from SB431542, Coco-Protein, Nicalin-Protein, Nomo-Protein, Folistatin or Lefty, more preferably being SB431542

Preferably in the Active Substance Combination (1) the molecular ratio of Gemcitabine: (B1) Nodal/Activin-Inhibitor is selected from a ratio of 1:0.0001-1.0, e.g., the ratio may be selected from a ratio of 1:0.0001-1.0, a ratio of 1:0.0005-1.0, from a ratio of 1:0.001-1.0, from a ratio of 1:0.005-1.0, from a ratio of 1:0.01-1.0, from a ratio of 1:0.05-1.0, from a ratio of 1:0.1-1.0, from a ratio of 1:0.5-1.0, or from a ratio of 1:0.0001-1.0, or may be selected from a ratio of 1:0.0001-0.75, from a ratio of 1:0.0001-0.75, from a ratio of 1:0.0001-0.5, from a ratio of 1:0.0001-0.1, from a ratio of 1:0.0001-0.05, from a ratio of 1:0.0001-0.01, from a ratio of 1:0.0001-0.005, from a ratio of 1:0.0001-0.001, or from a ratio of 1:0.0001-0.0005.

In the above defined exemplary Active Substance Combination (1) or definitions related thereto, the nucleoside analog gemcitabine may be replaced by any of the nucleoside analogs as defined herein or a combination of these nucleoside analogs as defined above. Alternatively, in the above defined exemplary Active Substance Combination (1) or definitions related thereto, the nucleoside analog gemcitabine may be replaced by any of the further anti-metabolite agents as defined above or a combination of these further anti-metabolite agents as defined above.

One other preferred embodiment of the invention refers to an Active Substance Combination (2) according to the invention comprising
(A) Gemcitabine
and
(B2a) at least one SHH inhibitor, preferably selected from Cyclopamine, Cyclopamine-KAAD, Jerkin, CUR 61414, Forskolin, SANT-1, Arsenic Trioxide, or CUR-0199691, more preferably being Cyclopamine;
   and
(B2b) at least one mTOR inhibitor, preferably selected from Rapamycin , Temsirolimus (CCI-779), Everolimus (RAD 001), Deforolimus (AP 23573) or TAFA 93, more preferably being Rapamycin.
   Preferably the Active Substance Combination (2) is selected from a combination of
Gemcitabine, Rapamycin and Cyclopamine,
Gemcitabine, Rapamycin and Cyclopamine-KAAD,
Gemcitabine, Rapamycin and Jervine,
Gemcitabine, Rapamycin and CUR 61414,
Gemcitabine, Rapamycin and Forskolin,
Gemcitabine, Rapamycin and SANT-1,
Gemcitabine, Rapamycin and Arsenic Trioxide,
Gemcitabine, Rapamycin and CUR-0199691,
Gemcitabine, Temsirolimus (CCI-779) and Cyclopamine,
Gemcitabine, Temsirolimus (CCI-779) and Cyclopamine-KAAD,
Gemcitabine, Temsirolimus (CCI-779) and Jervine,
Gemcitabine, Temsirolimus (CCI-779) and CUR 61414,
Gemcitabine, Temsirolimus (CCI-779) and Forskolin,
Gemcitabine, Temsirolimus (CCI-779) and SANT-1,
Gemcitabine, Temsirolimus (CCI-779) and Arsenic Trioxide,
Gemcitabine, Temsirolimus (CCI-779) and CUR-0199691,
Gemcitabine, Everolimus (RAD 001) and Cyclopamine,
Gemcitabine, Everolimus (RAD 001) and Cyclopamine-KAAD,
Gemcitabine, Everolimus (RAD 001) and Jervine,
Gemcitabine, Everolimus (RAD 001) and CUR 61414,
Gemcitabine, Everolimus (RAD 001) and Forskolin,
Gemcitabine, Everolimus (RAD 001) and SANT-1,
Gemcitabine, Everolimus (RAD 001) and Arsenic Trioxide,
Gemcitabine, Everolimus (RAD 001) and CUR-0199691,
Gemcitabine, Deforolimus (AP 23573) and Cyclopamine,
Gemcitabine, Deforolimus (AP 23573) and Cyclopamine-KAAD,
Gemcitabine, Deforolimus (AP 23573) and Jervine,
Gemcitabine, Deforolimus (AP 23573) and CUR 61414,
Gemcitabine, Deforolimus (AP 23573) and Forskolin,
Gemcitabine, Deforolimus (AP 23573) and SANT-1,
Gemcitabine, Deforolimus (AP 23573) and Arsenic Trioxide,
Gemcitabine, Deforolimus (AP 23573) and CUR-0199691,
Gemcitabine, TAFA 93 and Cyclopamine,
Gemcitabine, TAFA 93 and Cyclopamine-KAAD,
Gemcitabine, TAFA 93 and Jervine,
Gemcitabine, TAFA 93 and CUR 61414,
Gemcitabine, TAFA 93 and Forskolin,
Gemcitabine, TAFA 93 and SANT-1,
Gemcitabine, TAFA 93 and Arsenic Trioxide, or
Gemcitabine, TAFA 93 and CUR-0199691.

In one embodiment the Active Substance Combinations (2) listed above are consisting of the active substances listed in each combination.

One embodiment of the invention refers to an Active Substance Combination (2) according to the invention consisting of
(A) Gemcitabine
   and
(B2a) at least one SHH inhibitor, preferably selected from Cyclopamine, Cyclopamine-KAAD, Jervine, CUR 61414, Forskolin, SANT-1, Arsenic Trioxide, or CUR-0199691, more preferably being Cyclopamine;
   and
(B2b) at least one mTOR inhibitor, preferably selected from Rapamycin , Temsirolimus (CCI-779), Everolimus (RAD 001), Deforolimus (AP 23573) or TAFA 93, more preferably being Rapamycin.

Preferably in this Active Substance Combination (2) the molecular ratio of Gemcitabine: (B2a) SHH-Inhibitor: (B2b) mTor-Inhibitor is selected from a ratio of 1:0.001-1.1:0.0001-0.01, e.g., may be selected from a ratio of 1:0.001-1.1:0.0001-0.01, a ratio of 1:0.01-1.1:0.0001-0.01, a ratio of 1:0.1-1.1:0.0001-0.01, a ratio of 1:0.001-1.1:0.001-0.01, a ratio of 1:0.01-1.1:0.001, or a ratio of 1:0.1-1.1:0.001-0.01.

In the above defined exemplary Active Substance Combination (2) or definitions related thereto, the nucleoside analog gemcitabine may be replaced by any of the nucleoside analogs as defined herein or a combination of these nucleoside analogs as defined above. Alternatively, in the above defined exemplary Active Substance Combination (2) or definitions related thereto, the nucleoside analog gemcitabine may be replaced by any of the further anti-metabolitic agents as defined above or a combination of these further anti-metabolitic agents as defined above.

Also included as a further aspect of this invention are Active Substance Combinations (X) comprising
(A) Gemcitabine
   and
(B2a) at least one SHH inhibitor, preferably selected from Cyclopamine, Cyclopamine-KAAD, Jervine, CUR 61414, Forskolin, SANT-1 or Arsenic Trioxide, more preferably being Cyclopamine.
   Preferably this Active Substance Combination (X) is selected from a combination of
Gemcitabine and Cyclopamine,
Gemcitabine and Cyclopamine-KAAD,
Gemcitabine and Jervine,
Gemcitabine and CUR 61414,
Gemcitabine and Forskolin,
Gemcitabine and SANT-1,
Gemcitabine and Arsenic Trioxide, or
Gemcitabine and CUR-0199691.

Preferably in this Active Substance Combination (X) the molecular ratio of Gemcitabine: SHH-Inhibitor is selected from 1:0.001-1.0, e.g., the ratio may be selected from a ratio of 1:0.001-1.0, from a ratio of 1:0.005-1.0, from a ratio of 1:0.01-1.0, from a ratio of 1:0.05-1.0, from a ratio of 1:0.1-1.0, from a ratio of 1:0.5-1.0, or from a ratio of 1:0.001-1.0, or may be selected from a ratio of 1:0.0001-0.75, from a ratio of 1:0.001-0.75, from a ratio of 1:0.001-0.5, from a ratio of 1:0.001-0.1, from a ratio of 1:0.001-0.05, from a ratio of 1:0.001-0.01, or from a ratio of 1:0.001-0.005.

In the above defined exemplary Active Substance Combination (X) or definitions related thereto, the nucleoside analog gemcitabine may be replaced by any of the nucleoside analogs as defined herein or a combination of these nucleoside analogs as defined above. Alternatively, in the above defined exemplary Active Substance Combination (X) or definitions related thereto, the nucleoside analog gemcitabine may be replaced by any of the further anti-metabolitic agents as defined above or a combination of these further anti-metabolitic agents as defined above.

Also included as a further aspect of this invention are Active Substance Combinations (Y) comprising
(A) Gemcitabine
   and
(B2b) at least one mTOR inhibitor, preferably selected from Rapamycin, Temsirolimus (CCI-779), Everolimus (RAD 001), Deforolimus (AP 23573) or TAFA 93, more preferably being Rapamycin.
   Preferably this Active Substance Combination (Y) is selected from a combination of
Gemcitabine and Rapamycin,
Gemcitabine and Temsirolimus (CCI-779),
Gemcitabine and Everolimus (RAD 001),
Gemcitabine and Deforolimus (AP 23573), or
Gemcitabine and TAFA 93.

Preferably in this Active Substance Combination (Y) the molecular ratio of Gemcitabine: mTor-Inhibitor is selected from 1:0.0001-0.01, e.g., the ratio may be selected from a ratio of 1:0.0001-0.01, from a ratio of 1:0.0005-0.01, from a ratio of 1:0.001-0.01, or from a ratio of 1:0.005-0.01, or may be selected from a ratio of 1:0.0001-0.0005, from a ratio of 1:0.0001-0.001, or from a ratio of 1:0.0001-0.005.

In the above defined exemplary Active Substance Combination (Y) or definitions related thereto, the nucleoside analog gemcitabine may be replaced by any of the nucleoside analogs as defined herein or a combination of these nucleoside analogs as defined above. Alternatively, in the above defined exemplary Active Substance Combination (Y) or definitions related thereto, the nucleoside analog gemcitabine may be replaced by any of the further anti-metabolitic agents as defined above or a combination of these further anti-metabolitic agents as defined above.

Another aspect of the invention refers to a medicament comprising an active substance combination according to the invention (as described above) and optionally at least one or more physiologically acceptable excipients. Specifically this refers to medicaments comprising an active substance combination (1) according to the invention or to medicaments comprising an active substance combination (2) according to the invention. It also refers to medicaments comprising an active substance combination (X) or (Y) according to the invention.

Another aspect of the invention refers to the use of an active substance combination according to invention (as described above) for the treatment of cancer, preferably for the treatment of epithelial tumours or for the treatment of pancreatic cancer, ovarian cancer, bladder cancer, colon cancer, breast cancer, leukemia, lung cancer, or brain tumour, more preferably for the treatment of epithelial cancer or for the treatment of pancreatic cancer, colon cancer, breast cancer, leukemia, or non small cell lung cancer (adeno carcinoma). Specifically this use refers to active substance combination (1) according to the invention or this use refers to active substance combination (2) according to the invention. It also refers to the use of active substance combinations (X) or (Y) according to the invention.

Another aspect of the invention refers to the use of an active substance combination according to invention (as described above) for the production of a medicament for the treatment of cancer, preferably for the treatment of epithelial tumours or for the treatment of pancreatic cancer, ovarian cancer, bladder cancer, colon cancer, breast cancer, leukemia, lung cancer, or brain tumour, more preferably for the treatment of epithelial cancer or for the treatment of pancreatic cancer, colon cancer, breast cancer, leukemia, or non small cell lung cancer (adeno carcinoma). Specifically this use refers to active substance combination (1) according to the invention or this use refers to active substance combination (2) according to the invention. It also refers to the use of active substance combinations (X) or (Y) according to the invention.

A further aspect of the invention refers to the use of an active substance combination according to the invention as described above in the production of a medicament for the chemotherapeutic treatment of cancer. Specifically this use refers to active substance combination (1) according to the invention or this use refers to active substance combination (2) according to the invention. It also refers to the use of active substance combinations (X) or (Y) according to the invention.

Another aspect of the invention refers to the use of an active substance combination according to the invention as described above for chemotherapy, especially in relation to cancer. Specifically this use refers to active substance combination (1) according to the invention or this use refers to active substance combination (2) according to the invention. It also refers to the use of active substance combinations (X) or (Y) according to the invention.

“Chemotherapy” in the sense of this invention is defined as the use of a chemotherapeutic drug or active substance combination for the treatment of cancer or tumours or malign neoplasia respectively.

The various uses according to the invention described above are preferably conducted by using the active substance combination according to the invention as described above in form of a medicament or pharmaceutical formulation comprising the active substance combination according to the invention. Specifically this refers also to active substance combinations (1) according to the invention or to active substance combinations (2) according to the invention. It also refers to active substance combinations (X) or (Y) according to the invention.

According to the various embodiments or aspects of this invention, the active substance combinations or the pharmaceutical compositions or medicaments comprising them, may be administered in unit dosage form, intestinally, enterally, parenterally or topically, orally, subcutaneously, intranasally, by inhalation, by oral absorption, intravenously, intramuscularly, percutaneously, intraperitoneally, rectally, intravaginally, transdermally, sublingually, buccally, orally transmucosally. Administrative dosage forms may include the following: tablets, capsules, dragees, lozenges, patches, pastilles, gels, pastes, drops, aerosols, pills, powders, liquors, suspensions, emulsions, granules, ointments, creams, suppositories, freeze-dried injections, injectable compositions, in food supplements, nutritional and food bars, syrups, drinks, liquids, cordials etc, which could be regular preparation, delayed-released preparation, controlled-released preparation and various micro-granule delivery system, in food supplements, nutritional and food bars, syrups, drinks, liquids, cordials. In case of tablet, various carriers known in the art may be used, e.g., diluent and resorbent such as starch, dextrin, calcium sulfate, kaolin, microcrystalline cellulose, aluminium silicate, etc; wetting agent and adhesives such as water, glycerin, polyethylene glycol, ethanol, propanol, starch mucilage, dextrin, syrup, honey, glucose solution, acacia, gelatin, carboxymethylcellulose sodium, shellac, methylcellulose, potassium phosphate, polyvinylpyrrolidone, etc; disintegrating agent, such as dried starch, alginate, agar powder, laminaran, sodium bicarbonate and citric acid, calcium carbonate, polyoxyethylene sorbitol aliphatic ester, lauryl sodium sulfate, methylcellulose, ethylcellulose, lactose, sucrose, maltose, mannitol, fructose, various disaccharides and polysaccharides etc; disintegration inhibiting agent, such as sucrose, tristearin, cacao butter, hydrogenated oil, etc; absorption accelerator, such as quaternary ammonium salt, lauryl sodium sulfate, etc; lubricant, such as talc, silica, corn starch, stearate, boric acid, fluid wax, polyethylene, etc. The tablet may be further formulated into coated tablet, e.g., sugar-coated tablet, film-coated tablet, enteric-coated tablet, or double-layer tablet and multi-layer tablet. In the case of pill, various carriers known in the art may be used, e.g., diluent and resorbent, such as glucose, lactose, starch, cacao butter, hydrogenated vegetable oil, polyvinylpyrrolidone, kaolin, talc, etc; adhesives, such as acacia, bassora gum, gelatin, ethanol, honey, liquid sugar, rice paste or flour paste, etc; disintegrating agent, such as agar powder, dried starch, alginate, lauryl sodium sulfate, methylcellulose, ethylcellulose. In case of suppository, various carriers known in the art may be used, e.g., polyethylene, lecithin, cacao butter, higher alcohols, esters of higher alcohols, gelatin, semi-synthetic glyceride, etc. In the case of capsule, it may be prepared by mixing said active substance combinations as active ingredient with the above mentioned carriers, followed by placing the mixture into a hard gelatin capsule or soft capsule. Also, said active substance combinations may be applied in the following dosage forms: microcapsules, suspension in an aqueous phase, hard capsule, or injection. In the case of injection, such as liquor, emulsion, freeze-dried injection, and suspension, all the diluents common in the art may be used, e.g., water, ethanol, polyethylene glycol, propylene glycol, oxyethylated isostearyl alcohol, polyoxidated isostearyl alcohol, polyoxyethylene sorbitol aliphatic ester, etc. In addition, in order to obtain isotonic injection, a suitable amount of sodium chloride, glucose or glycerin may be added into the preparation, as well as regular cosolvent, buffer, pH adjusting agent, etc. In addition, coloring agent, antiseptic, perfume, correctives, food sweetening agent or other materials may be added to the pharmaceutical preparation if necessary. Specifically these above mentioned medicaments or pharmaceutical formulations refer to active substance combinations (1) according to the invention or to active substance combinations (2) according to the invention. They also refer to active substance combinations (X) or (Y) according to the invention.

In certain embodiments a formulation or pharmaceutical composition according to the invention contains the active substance combination according to the invention as well as optionally at least one auxiliary material and/or additive and/or optionally another active ingredient. Specifically this refers also to active substance combinations (1) according to the invention or to active substance combinations (2) according to the invention. It also refers to active substance combinations (X) or (Y) according to the invention.

In a very preferred embodiment of the medicament according to the invention or of the uses (using a medicament or pharmaceutical formulation comprising an active substance combination according to the invention) according to the invention the medicament or pharmaceutical formulation is in the form of an injectable liquid, or a physiologically acceptable injectable liquid like a physiological saline solution comprising the active substance combination, to be used for intravenous application. Most preferably the active substance composition is dissolved in 0.9% sodium chloride in water without further excipients or additives, desirably with a maximum content of 40 mg/ml of the nucleoside analog (preferably Gemcitabine). In another very preferred embodiment of the medicament according to the invention or of the uses (using a medicament or pharmaceutical formulation comprising an active substance combination according to the invention) according to the invention the medicament or pharmaceutical formulation is in the form of a dry powder that can be reconstituted with injectable liquid, or with a physiologically acceptable injectable liquid like a physiological saline solution to comprise the active substance combination, to be used for intravenous application. Most preferably the active substance composition is ready to be dissolved in 0.9% sodium chloride in water without further excipients or additives, desirably up to a maximum content of 40 mg/ml of the nucleoside analog (preferably Gemcitabine). Specifically this refers also to active substance combinations (1) according to the invention or to active substance combinations (2) according to the invention. It also refers to active substance combinations (X) or (Y) according to the invention.

A further aspect of this invention also refers to a method of treating a neoplasm or cancer in a mammal in need thereof, which comprises providing to said mammal an effective amount of an active substance combination according to the invention. In a desirable embodiment of this method of treating a mammal the different substances of the active substance combination according to the invention as described above are applied either together or administered as part of the same composition, or may be administered separately, at the same or at separate times, in the same therapeutic regimen. This therapeutic regimen may be a chemotherapeutic treatment of a neoplasm or cancer in a mammal. In a preferred embodiment the chemotherapeutic regimen may include as an additional step also the treatment with a platin derivative chemotherapeutic like cisplatin. Specifically all of the above aspects relating to a method of treating a neoplasm in a mammal refer also to active substance combinations (1) according to the invention or to active substance combinations (2) according to the invention. They also refer to active substance combinations (X) or (Y) according to the invention.

The method of treating a mammal according to the invention described above is in some embodiments conducted by applying the active substance combination according to the invention as described above in form of one medicament or pharmaceutical formulation comprising the active substance combination according to the invention. In other specific embodiments of the method of treating a mammal according to the invention the method is conducted by applying the substances of the active substance combination according to the invention separately in form of medicaments or pharmaceutical formulations comprising separate substances of the active substance combination according to the invention. Possibilities of appropriate pharmaceutical formulations and medicaments are described above and also well-known in the art. Especially preferred is a medicament or pharmaceutical formulation in the form of one or more physiological saline solutions, comprising the active substance combination or one or more of the separate substances of the active substance combination with a maximum content of 40 mg/ml of the nucleoside analog (preferably Gemcitabine). Again specifically this entire paragraph refers also to active substance combinations (1) according to the invention or to active substance combinations (2) according to the invention. It also refers to active substance combinations (X) or (Y) according to the invention.

In a preferred embodiment of the method of treating a neoplasm in a mammal according to the invention the neoplasm or cancer is an epithelial tumour or a pancreatic cancer, ovarian cancer, bladder cancer, colon cancer, breast cancer, leukemia, lung cancer, or a brain tumour, more preferably is epithelial cancer or pancreatic cancer, colon cancer, breast cancer, leukemia, or non small cell lung cancer (adeno carcinoma). In another preferred embodiment of the method of treating a neoplasm in a mammal according to the invention the mammal is a human, female or male, an adult or a child.

### FIGURES

The figures in the following section describing results of pharmacological trials are merely illustrative and the invention cannot be considered in any way as being restricted to these figures.

FIGS. 1-8: Triple active substance combination of Gemcitabine, the mTOR-Inhibitor Rapamycin and the SHH-inhibitor Cyclopamine

FIGS. 1A-1C: show the content of cancer stem cells (measured as “CD 133-content”) in the flow cytometry in tumour cell lines after 48 hours of in-vitro treatment in control sample, a sample additionally comprising Gemcitabine and a further sample, additionally comprising Gemcitabine, a SHH inhibitor and an mTOR inhibitor (CRG=combination of Cyclopamine (SHH inhibitor), Rapamycin (mTOR inhibitor) and Gemcitabine). As can be seen in the representative flow cytometry plots, Gemcitabine even increased the number of CD133⁺ cancer stem cells when compared to CRG or the control, whereas CRG significantly reduced the number of CD133⁺ cancer stem cells.

FIG. 2: shows the content of cancer stem cells (measured as “CD 133-content”) in the flow cytometry in tumour cell lines after 48 hours of in-vitro treatment. As can be seen, none of the investigated molecules (Gemcitabine, Rapamycin (mTOR inhibitor) and Cyclopamine (SHH inhibitor)) was capable of significantly reducing the number of CD133⁺ cancer stem cells when used separately. However, when the triple combination CRG (Cyclopamine, Rapamycin and Gemcitabine) was applied an almost complete elimination of CD133⁺ cancer stem cells could be accomplished and almost none of these cells were detectable in flow cytometry.

FIGS. 3A-3D: show in FIG. 3A the in vitro migratory of tumour cells after 48 hours of in-vitro treatment. As can be seen in combined therapy using all three substances (100 ng/ml Gemcitabine, 100 ng/ml Rapamycin (mTOR inhibitor), 10 μM Cyclopamine (SHH inhibitor)) drastically reduced the invasive capacity in this in vitro assay opposed to control or Gemcitabine alone. The experimental setup and the in vivo metastatic activity of tumour cells after 48 hours of in-vitro treatment is shown in FIG. 3B, assessing the validity of the assay by in vivo investigation of the metastatic activity of the treated cells following in vitro pre-treatment (see FIGS. 3C and 3D; white arrows indicate metastatic lesions). Gemcitabine was administered using the commercially available drug Gemzar™.

FIGS. 4A-4D: shows the “CD133 content” in the flow cytometry in primary cell lines from patients with pancreatic cancer after 48 hours of in-vitro treatment. FIGS. 4A-4D show the findings shown in FIGS. 1 to 3 in tumour cell lines, which were reproduced in primary patient tissue with viability tested with propidium iodide. As can be seen, FIG. 4B illustrates the treatment with Gemcitabine alone, FIG. 4C illustrates the viability of Propidium iodide, and FIG. 4D illustrates in vitro treatment with the combined therapy of 100 ng/ml Gemcitabine, 100 ng/ml Rapamycin (mTOR inhibitor), and 10 μM Cyclopamine (SHH inhibitor) led to the elimination of CD 133⁺ cancer stem cells.

FIGS. 5A-5B: depicts the tumorigenicity of tumour cells after in-vitro pre-treatment. The experimental setup is shown initially. In vitro pre-treatment using the combination of all three substances (100 ng/ml Gemcitabine, 100 ng/ml Rapamycin (mTOR inhibitor), and 10 μM Cyclopamine (SHH inhibitor)) led to a complete reversal of tumorigenicity opposed to control or separate treatment with the substances of the active substance combination or even with a double combination of Rapamycin and Cyclopamine.

FIGS. 6A-6B: mg/kg by oral gavages twice daily and Rapamycin was orally administered via the drinking water (5 mg/kg). As can be seen in FIG. 6B, no palpable tumour could be found in 80% of mice treated with the triple combination mentioned above.

FIGS. 7A-7B: depicts the tumour size in a mouse model after in-vivo treatment in the experiment shown in FIG. 6. As can be seen in FIG. 7B, tumour size was significantly smaller compared to control (P<0.05 Gemcitabine vs. Control and P<0.05 Targeted treatment vs. control). Importantly, while standard therapy with Gemcitabine resulted in a prolongation of median survival by 22 days (FIG. 8), combination treatment translated into a prolonged tumour- and metastasis-free survival of animals compared to control (FIG. 9). Intriguingly, the majority of the animals survived the extended follow-up period of 100 days.

FIG. 8: shows in a Kaplan-Meier-Analysis the incident free survival of mice after in-vivo treatment (survival gain by standard therapy) in the experiment shown in FIGS. 6 and 7. Standard therapy with Gemcitabine resulted in a prolongation of median survival by 22 days.

FIG. 9: likewise shows in a Kaplan-Meier-Analysis the incident free survival of mice after in-vivo treatment (survival gain by combination therapy as compared to standard therapy) in the experiment describes tumour incidents in a mouse model after in-vivo treatment, wherein tumour-bearing mice received either no treatment, or Gemcitabine alone, or in combination with Cyclopamine (SHH inhibitor) and Rapamycin (mTOR inhibitor). Gemcitabine was administered twice a week by intraperitoneal injections at 125 mg/kg BW. Cyclopamine was used as shown in FIGS. 6, 7 and 8. As can be seen, combination treatment with Gemcitabine, Cyclopamine (SHH inhibitor) and Rapamycin (mTOR inhibitor) led to a prolonged tumour- and metastasis-free survival of animals compared to control. Intriguingly, the majority of the animals survived the extended follow-up period of 100 days.

FIGS. 10-17: Double active substance combination of Gemcitabine and the Nodal/Activin inhibitor SB431542

FIGS. 10A-10C: shows the content of cancer stem cells (measured as “CD 133-content”) in the flow cytometry in tumour cell lines after 48 hours of in-vitro treatment. Separate treatment with 5 μM SB431542 alone (a Nodal/Activin inhibitor) already resulted in a significant reduction in CD133⁺ cancer stem cells in the experiments, whereas single treatment with 100 ng/ml Gemcitabine alone, FIG. 10B, did not. With treatment with the active substance combination of 100 ng/ml Gemcitabine and 5 μM SB431542, FIG. 10C, a complete elimination of CD133⁺ cancer stem cells could be achieved in vitro. FIG. 10A is a control.

FIG. 11: depicts the “CD133 content” in the flow cytometry in tumour cell lines after 48 hours of in-vitro treatment. As already shown in FIG. 10 separate treatment with 5 μM SB431542 alone (a Nodal/Activin inhibitor) already resulted in a significant reduction in CD133⁺ cancer stem cells in the experiments, whereas single treatment with 100 ng/ml Gemcitabine alone did not. With treatment with the active substance combination of 100 ng/ml Gemcitabine and 5 μM SB431542 a complete elimination of CD133⁺ cancer stem cells could be achieved in vitro. Use of an ALK5 inhibitor (SB-505124) or an ALK5 inhibitor (SB-505124) and Gemcitabine did not lead to significant reduction in CD133⁺ cancer stem cells. P<0.05 vs. inhibitor alone (using Mann-Whitney U test).

FIG. 12: shows the migratory activity in tumour cell lines after 48 hours of in-vitro treatment. The transmigratory activity as an important functional marker for the invasiveness of these cells was almost completely inhibited after combination therapy with the double combination of Gemcitabine and SB431542 (a Nodal/Activin inhibitor) opposed to control or single treatment with Gemcitabine.

FIGS. 13A-13D: depicts the “CD133 content” in the flow cytometry in fresh primary tumour cells from patients with pancreatic cancer after 48 hours of in-vitro treatment with viability tested with propidium iodide, FIG. 13C. As can be seen, in vitro treatment with the combined therapy of 100 ng/ml Gemcitabine and 5 μM SB431542 (a Nodal/Activin inhibitor) led to elimination of CD 133+ cancer stem cells FIG. 13D vs. Gemcitabine alone, FIG. 13B.

FIGS. 14A-14B: shows the tumorigenicity of tumour cells after in-vitro pre-treatment. The experiment was carried out after transplantation of cells pretreated with different sets of treatments in vitro in an orthotopic mouse model of pancreatic cancer. In-vitro pre-treatment with either 100 ng/ml Gemcitabine, or 5 μM SB431542 alone or the double active substance combination of Gemcitabine and SB431542 (a Nodal/Activin inhibitor) led to a complete reversal of tumorigenicity with the double combination and close to no effect of the substances alone.

FIGS. 15A-15B: depicts tumour incidents in a mouse model after in-vivo treatment, wherein seven days after orthotopic implantation of tumour cells, therapy with either Gemcitabine alone or in combination with targeted Nodal/Activin inhibition by SB431542 was initiated. None of the investigated mice treated with combination therapy showed evidence for tumour formation.

FIGS. 16A-16B: shows the tumour size in a mouse model after in-vivo treatment, wherein seven days after orthotopic implantation of tumour cells, therapy with either Gemcitabine alone or in combination with targeted Nodal/Activin inhibition by SB431542 was initiated. Contrary to the results in FIG. 15, tumour incidence was unaffected by Gemcitabine monotherapy, which merely resulted in reduction of tumour size.

FIG. 17: shows in a Kaplan-Meier-Analysis the incident free survival of mice after in-vivo treatment with Gemcitabine and Gemcitabine in combination with Nodal/Activin inhibitor SB431542. The long-time tumour- and metastasis-free survival of the animals receiving combination therapy was striking as compared to Gemcitabine alone treated mice.

FIGS. 18-26: Further results for double active and triple active substance combinations

FIGS. 18A-18A6: shows results of the in vitro evaluation of anti-cancer stem cell agents. In this experiment, potentially effective substances for the targeted elimination of CD133⁺ pancreatic cancer stem cells (CSC) were screened by means of flow cytometry following 48 hours of therapy. As can be seen, a marked enrichment of CD133⁺ cells following Gemcitabine therapy was observed in contrast to the control or further components administered (Cyclopamine (Cyclo), Cyclopamine (Cyclo) and Rapamycin (Rapa), Cyclopamine (Cyclo) and (Gemcitabine (G)), Rapamycin (Rapa) and Gemcitabine (G)). Best results were obtained for the inventive combination CRG (Cyclopamine, Rapamycin and Gemcitabine).

FIGS. 18B-18B4: shows results of the in vitro evaluation of anti-cancer stem cell agents. To assess whether mTOR signalling is indeed active in CSC histological analyses for the phosphorylation of p70s6-kinase were performed, a downstream target of mTOR that has been shown to be a reliable marker for the activity of the mTOR-pathway. As can be seen, mTOR signalling was active only in a small subset of cells including CD133+ CSC (FIG. 18B1; upper panel). Following mTOR inhibition by Rapamycin, phosphorylation of p-70-s6-kinase in CSC was profoundly reduced (FIG. 18B2; lower panel).

FIGS. 18C1-18C5: shows results of the in vitro evaluation of anti-cancer stem cell agents. For the identification of a subpopulation of cells enriched for CSC, pancreatic CSCs were clonally expanded as CSC-enriched spheres. Then floating spheres were treated in ultra low adhesion 6-well plates. After completion of the treatment, five randomly selected high-power fields were analyzed. As can be seen, Gemcitabine single-agent therapy resulted in a marked relative increase of CD133⁺ cells, consistent with a marked chemo-resistance of CD133⁺ cells while Cyclopamine or Rapamycin alone resulted in the reduction of tumour spheres with the resulting single cells eventually dying when kept in these specific stem cell conditions. Most importantly, CRG (Cyclopamine, Rapamycin and Gemcitabine) combination therapy demonstrated the strongest potential for tumoursphere depletion.

FIGS. 18D1-18D4: shows results of the in vitro evaluation of anti-cancer stem cell agents as described for FIG. 18C. In FIG. 18C, side populations (SP) were screened with different agents (verpamil, gemicitabine and CRG (Cyclopamine, Rapamycin, Gemcitabine and a control) under UV-A treatment. As can be seen, side population cells were unaffected by exposure to Gemcitabine whereas CRG triple therapy virtually depleted all SP cells.

FIGS. 18E1-1-18E3-3: shows results of the inhibition of SHH pathway in an experiment to eliminate metastatic activity. For this experiment, freshly isolated primary pancreatic cancer cells were subjected to treatment with an isocontrol (iso-PE), a control, gemicitabine, CRG (Cyclopamine, Rapamycin, and Gemcitabine). Consistent with the treatment effects observed for L3.6pl cells, Gemcitabine monotherapy tended to relatively enrich for the CSC fraction while combination therapy with CRG virtually abolished both CD133⁺ and CD24⁺CD44⁺EpCAM⁺ CSC populations, respectively.

FIG. 19A: shows a migration test of pancreatic cancer cells in a modified Boyden chamber assay using SDF-1 as the migratory stimulus. As can be seen, Gemcitabine, Rapamycin, both alone, and their combination (RG) were unable to significantly reduce the migratory activity. In contrast, Cyclopamine alone already showed a strong reduction, but only combination with Gemcitabine resulted in complete abrogation of functional capacity in vitro.

FIGS. 19B1-19B6: shows flow cytometry results of the migration test of migrating cancer stem cells. As can be seen, treatment with Gemcitabine enriches for metastatic CSC, also named migrating CSC, which are characterized by co-expression of CD133 and CXCR4, whereas only CG (Cyclopamine, and Gemcitabine; CG) and CRG (Cyclopamine, Rapamycin, and Gemcitabine; CRG), respectively, resulted in a complete elimination of this CD133⁺ CXCR4⁺ CSC subpopulation, providing a rationale for further evaluating these treatment modalities with respect to their anti-metastatic effect in vivo. RG is Rapamycin and Gemcitabine.

FIGS. 19C1-19C2 FIG. 19C: shows results of a histological analysis of a systemic infusion assay, wherein pretreated and Qtracker-labeled cells were systemically infused and seeded cells were tracked using near infrared scanning of the lungs as the primary target organ. While all mice receiving Gemcitabine-pretreated cells showed evidence for metastasis, metastatic spread tended to be reduced in Cyclopamine-pretreated cells Importantly, combination of Gemcitabine and Cyclopamine further and significantly reduced metastatic activity while triple CRG therapy resulted in complete loss of metastatic activity in vivo.

FIG. 20A: shows the experimental setup for a test on loss of tumorigenicity following in vitro pretreatment. Identical numbers of L3.6pl pancreatic cancer cells are either exposed to Gemcitabine alone, one of the stem cell pathway inhibitors alone, a combination of the inhibitors, or a combination of all three treatments. After 4 days of pretreatment, the surviving cells are orthotopically implanted into the pancreas, which receive no further in vivo treatment. Tumorigenicity is then first determined by PET scans on day 30 and by final macroscopic and microscopic histological evaluation on day 35.

FIGS. 20B-20C: shows determination of tumorigenicity by PET scans on day 30 (FIGS. 20B, 20C; left panel) and the final macroscopic and microscopic histological evaluation on day 35 (FIGS. 20B, 20C; right panel). The combination therapy with Cyclopamine and Rapamycin showed a trend to a lower tumour take rate, which is consistent with above in vitro findings demonstrating no complete abrogation of the CSC population but a significant reduction and already a synergistic effect. The CRG combination therapy resulted in complete abrogation of tumorigenicity, clearly involving a synergistic effect.

FIG. 20D: shows the statistical evaluation of the results as shown in FIG. 20B (see above). Surprisingly, neither Cyclopamine nor Rapamycin (mTOR inhibitor) monotherapy significantly affected tumour take rates as compared to Gemcitabine (SHH inhibitor)

FIG. 21A: shows the experimental setup of an experiment to test the identified combination therapy in a clinically most relevant setting. Therefore, a model of established orthotopic pancreatic cancer was used. One week (7 days) after cell implantation, the tumour take rate was confirmed and tumour-bearing mice were randomized for treatment. Cyclopamine and Rapamycin were administered for only two weeks (between day 7 and 21) to minimize potential stem cell-associated side effects. Gemcitabine was given for a prolonged period of 11 weeks in analogy to clinical practice. Tumour volume was non-invasively measured on day 42, and was controlled via MR imaging on day 49. Based on these positive results, the experiment was continued until day 100.

FIG. 21B: shows the determination of the tumour volume by non-invasively measuring on day 42 using a calliper. CRG significantly decreased tumor size, whereas gemicitabine still showed a considerable tumor size.

FIG. 21C: shows the determination of the tumour volume by control via MR imaging on day 49. CRG significantly decreased tumor size, whereas gemicitabine still showed a considerable tumor size.

FIG. 21D: shows the results of a white blood cell counts. As can be seen, these white blood cell counts showed no evidence for undesired effects of the stem cell inhibitors, particularly the combination CRG, on the hematopoietic system.

FIG. 21E: shows the tumour take rate on day 100. Intriguingly, tumour take rate on day 100 was dramatically reduced in the CRG group as compared to Gemcitabine alone. All control animals bore large, life-limiting tumours and succumbed within one month after tumour implantation. Gemcitabine alone significantly prolonged survival of the animals due to inhibition of tumour growth. However, the animals' median survival was still severely limited with 57 days due to the 100% tumour take rate. Intriguingly, for the CRG group, long-term survival was significantly better compared to Gemcitabine alone.

FIG. 21F: shows a Kaplan-Mayer analysis of the data shown in FIG. 21E, confirming these results. Only one CRG animal carried a detectable tumour and had to be prematurely sacrificed on day 44. One death on day 8 was related to the oral feeding procedure and therefore censored. The other two deaths in the CRG group were also unrelated to tumour growth rather than related to local infections (n=1) or remained obscure without apparent pathologies during necropsy (n=1). In summary, out of 11 animals, 63% of the CRG animals reached the 100-day follow-up time point whereas none of the animals in the Gemcitabine alone group survived beyond day 71. As a consequence, CRG not only significantly prolonged lifetime but also aspparently abrogated cancer stem cells (CSC).

FIGS. 22A-22B: shows results of targeting the tumorigenic population in Pancreatic Cancer by the inhibition of Activin/Nodal Signaling. In an experiment using cells in culture subjected to extracellular antagonists of Nodal (Lefty) (and Activin A (Follistatin)) Lefty decreased the CD133 positive population to a significant extent, FIG. 22B vs. Control, FIG. 22A.

FIG. 22C: depicts results of targeting the tumorigenic population in Pancreatic Cancer by the inhibition of Activin/Nodal Signaling with Lefty and Folistatin. Both Lefty as a Nodal inhibitor and Folistatin as an inhibitor of Activin signaling resulted in a reduction of CD133 positive CSC that was similar to the reduction seen for Lefty (see also FIG. 23). Even more importantly, like treatment with SB431542, an Activin/Nodal inhibitor, the treatment effect was by far more effective in depleting CSCs when cytostatic therapy was added.

FIG. 23: depicts further results of targeting the tumorigenic population in Pancreatic Cancer by the inhibition of Activin/Nodal Signaling with Lefty and Folistatin. Both Lefty as a Nodal inhibitor and Folistatin as an inhibitor of Activin signaling resulted in a reduction of CD133 positive CSC that was similar to the reduction seen for Lefty (see also FIG. 22B).

FIGS. 24A-24D: shows the results of a treatment of cells with SB505124, an inhibitor of ALK-5 is depicted in FIG. 24C, a preferential inhibitor of ALK-5 with lower affinity to ALK-4 and ALK-7, the receptor for TGF-β to clarify whether there is a significant contribution of TGF-β to these effects. However, neither alone nor in combination with Gemcitabine an effect of SB505124 on the CD133 content of L3.6pl cells was observed. (The graph, FIG. 24D, shows the CD133+ CSC content (% of control) for the inhibitors.) The structure of compound SB431542, an inhibitor of ALK-4, -5, and -7 is depicted in FIG. 24B.

FIG. 25: depicts the content of CD24/CD44 double positive cells in an AsPc1 cell line after a 48 hour treatment, as Cancer Stem Cells (CSC) in Pancreatic Cancer have also been identified by combined expression of CD44 and CD24. Whereas treatment with Gemcitabine alone did not lead to any effect, treatment with Gemcitabine and SB431542 led to a significant reduction of CD24/CD44 double positive cells in an AsPc1 cell line.

FIGS. 26A-26G: shows the results of a combined therapy with SB431542 and Gemcitabine. FIG. 26A is a control. As can be seen, combined therapy with SB431542 and Gemcitabine permanently affects Cancer Stem Cells, FIG. 26E. At 24 hours, FIG. 26F, the CD133 content of cells treated with the small molecule inhibitor alone already started to approach control level while it was not altered as compared to the base level in cells treated with Gemcitabine and SB431542. Even after 48 h, FIG. 26G no rebound in the CD133 content was observed after combined treatment, as opposed to SMI-treatment only which even resulted in an enrichment for CD133 compared to base level. As the precise fate of CD133-positive cells during treatment with antagonists of Activin or Nodal was to be assessed, the cell cycle of these cells was investigated using a BrDU flow kit. While treatment with SB431542, FIG. 26B, resulted only in a modest induction of apoptosis in this subset of cells, the combined treatment with Gemcitabine accounted for massive apoptosis. The results 24 hours and 48 hours after removal of treatment with SB431542 are depicted in FIG. 26C and FIG. 26D, respectively.

FIGS. 27A-D: show that an innovative in vitro culture system to functionally enrich for pancreatic cancer stem cells was established. Compared to standard culture conditions (adherent, FIG. 27A), anchorage-independent and serum-free culture (plus additional specific factors to enhance self-renewal of cancer stem cells) of primary cancer cells leads to the functional enrichment for cancer stem cells independent of surface markers (Spheres, FIG. 27B). These spheres contain more differentiated, cytokeratin 19 positive cells in the outer rim, while undifferentiated cells mostly reside in the center of the spheres (lower right panel, FIG. 27D).

FIGS. 28A-D: shows Nodal signaling components overexpressed in hPaCSC, and that expression of Nodal, its cofactor (cripto) and Activin is very low under sphere conditions. Enrichment for cancer stem cells in sphere conditions results in very high expression of nodal, cripto and activin (80 to 100× higher). (hPaCSC=primary human pancreatic cancer stem cells).

FIGS. 29A-29B: shows that inhibition of Nodal/Activin signaling using the small molecule inhibitor SB431542, which blocks Alk4/7 (Nodal/Activin) and Alk5 (TGF-beta, low affinity). The SB505124, an inhibitor of the Alk5 receptor was used to demonstrate that the inhibitory effects of SB431542 were exclusively mediated through Alk4/7.

FIGS. 30A-30C: shows that treatment of cells with recombinant Nodal in a dose-dependent manner results in increased phosphorylation of downstream Smad2 (Experimental setup: adherent/spheres). Pretreatment of the cells with the Alk4/7 inhibitor SB431542 abrogated this increase in pSmad2 indicating that nodal acts through Alk4/7.

FIGS. 31A-D: show a Gain of function by Nodal stimulation—Sphere derived cells (185s) bear a higher colony forming capacity as compared to adherent cells (185) as evidence for an enrichment in cancer stem cells (in a soft agar assay 14d). Spheres derived from primary tumors (185 s) respond to nodal treatment with a further increase in colony formation.

FIGS. 32A-32C FIG. 32: shows that the loss of function by blockade of the Alk4/7 receptor (binding both Nodal and Activin)—Addition of the Alk4/7 receptor inhibitor SB431542 in a dose-dependent manner reduced sphere formation in primary human pancreatic cancer cells. Treatment did not affect cell viability as evidenced by dapi staining (right panel, FIG. 32C).

FIG. 33: In vivo treatment with the Alk4/7 inhibitor SB431542. This illustrates Nodal/Activin inhibition to eliminate primary human pancreatic CSC in vivo. Tumor tissue from patient with pancreatic cancer was implanted into immunocompromised mice. Treatment was started on day 7 after implantation. Mice were randomized to gemcitabine alone or gemcitabine plus SB431542. Tumor growth was assessed on day 28.

FIG. 34: shows an example of a tumor treated with SB431542. This illustrates Nodal/Activin inhibition to eliminate primary human pancreatic CSC in vivo. Displayed is a tumor with strong response to gemcitabine (debulking) However, addition of SB431542 results in a further regression of the tumors Importantly, the cancer stem cell content was significantly reduced indicating targeting of the cancer stem cell by SB431542 (data not shown).

FIGS. 35A-B: shows a safety study for SB431542. The addition of SB431542 did not result in severe side effects as evidenced by lack of change in body weight and white blood cell counts (WBC). This illustrates Nodal/Activin inhibition in vivo.

FIG. 36A-C: shows in vivo treatment of primary pancreatic cancer tumors using the triple combination treatment using an hedgehog inhibitor (cyclopamine or CUR199691) and the mTOR inhibitor rapamycine on top of gemcitabine treatment (standard care). Tumor tissue from patient with pancreatic cancer was implanted into immunocompromised mice. Treatment was started on day 21 after implantation. Mice were randomized to gemcitabine alone or combination therapy. Tumor growth was assessed up to 100 days. While tumors in the gemcitabine group demonstrated virtually unrestricted growth, tumors in the triple combination treatment groups regressed and, most importantly and most prominently, after withdrawal of all therapies in mice treated with the hedgehog inhibitor CUR199691 and the mTOR inhibitor rapamycine on top of gemcitabine treatment did not relapse as evidence for cure from pancreatic cancer.

FIG. 37: shows a safety study for triple combination treatment. The addition of one hedgehog inhibitor (cyclopamine or CUR199691) and the mTOR inhibitor rapamycine did not result in severe side effects as evidenced by lack of change in body weight.

FIG. 38: shows a safety study for triple combination treatment. The addition of a hedgehog inhibitor (cyclopamine or CUR199691) and the mTOR inhibitor rapamycine did not result in severe side effects as evidenced by lack of change in the white blood cell count (WBC).

FIGS. 39A-39D3: show the in vivo triple therapy results in tumor regression in primary human pancreatic cancer. Depiction of the experimental setup, FIG. 39A. Tumor volume during 49 days of follow-up for fast growing (left panel, FIG. 39B1) and slower growing tumors (right panel, FIG. 39B2). Survival times for the 3 treatment groups are illustrated as Kaplan-Meier survival curves (gemcitabine, n=12; CycloRG, n=13; CurRG, n=14; both P<0.001 vs. gemcitabine), FIG. 39C. Phenotyping of in vivo treated tumors on day 40 following implantation according to allocated treatment, FIG. 39D1, Gemcitabine; FIG. D2, Gemcitabine, Rapa, and Cyclopamine; and FIG. D3, Gemcitabine, Rapa and CUR199691. The cells were first gated for exclusion of 7AAD (not shown) and then for expression of Ep-CAM, followed by double staining or CD133/CD44 and CD44/CD24, respectively.

FIG. 40: shows that Alk4/7 binds nodal (requires the presence of the co-receptor cripto-1, which is also expressed in cancer stem cells) and activin. Specific inhibition of nodal by lefty and activin by folistatin, respectively, resulted in a reduction of CD133 positive cancer stem cells. The combination of lefty and folistatin showed additive activity. In combination with gemcitabine, the CD133 positive cancer stem cells were virtually eliminated. These data indicated that both nodal and lefty activate the Alk4/7 receptors in cancer stem cells and that their combined blockade is most effective in eliminating epithelial cancer stem cells.

FIG. 41 is an illustration of the Sonic Hedgehog Pathway scheme.

FIG. 42 is a scheme illustrating the mTOR pathway.

FIG. 43 is a scheme illustrating the Nodal signalling pathway.

### EXAMPLES

The examples in the following section describing pharmacological trials are merely illustrative and the invention cannot be considered in any way as being restricted to these examples.

### Example 1

### Triple Active Substance Combination of Gemcitabine, the mTOR-Inhibitor Rapamycin and the SHH-inhibitor Cyclopamine

### Example 1.1

### In-Vitro: Content of CSC by Flow Cytometry in Tumour Cell Lines

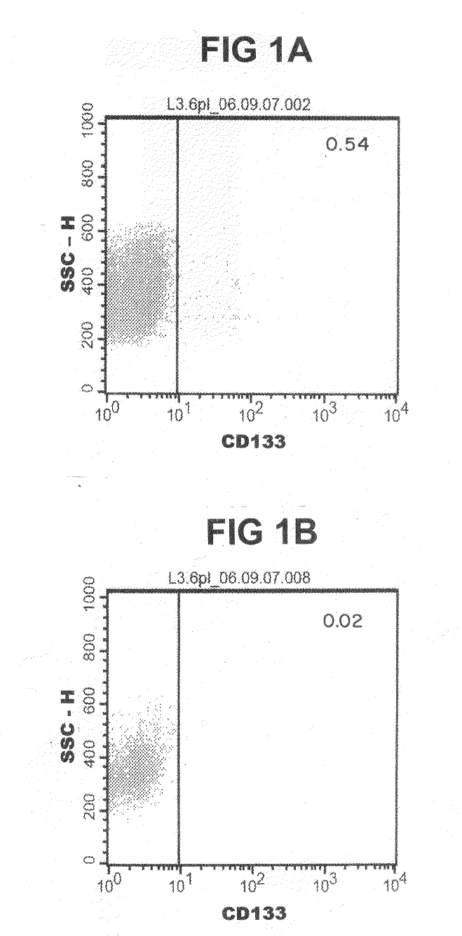

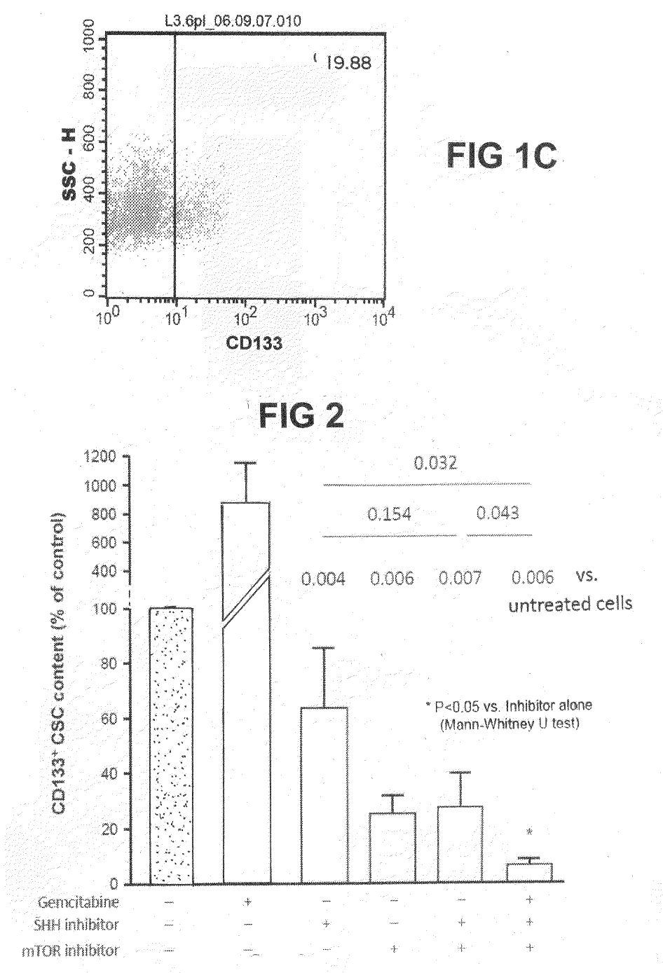

As a first step the content of cancer stem cells (measured as “CD 133-content”) in the whole population of tumour cells was measured by flow cytometry (FIGS. 1A-1C). The tumour cells then were either not treated or treated for 48 hours with 100 ng/ml Gemcitabine, 100 ng/ml Rapamycin, 10 μM Cyclopamine or a triple combination of these inhibitors in the above given concentration. None of the investigated molecules (Gemcitabine, Rapamycin and Cyclopamine) was capable of significantly reducing the number of CD133⁺ cancer stem cells when used separately (FIG. 2). However, when the triple combination was applied an almost complete elimination of CD133⁺ cancer stem cells could be accomplished and almost none of these cells were detectable in flow cytometry (FIGS. 1A-1C and FIG. 2).

### Example 1.2

### In-Vitro: Transmigratory Activity of Tumour Cell Lines

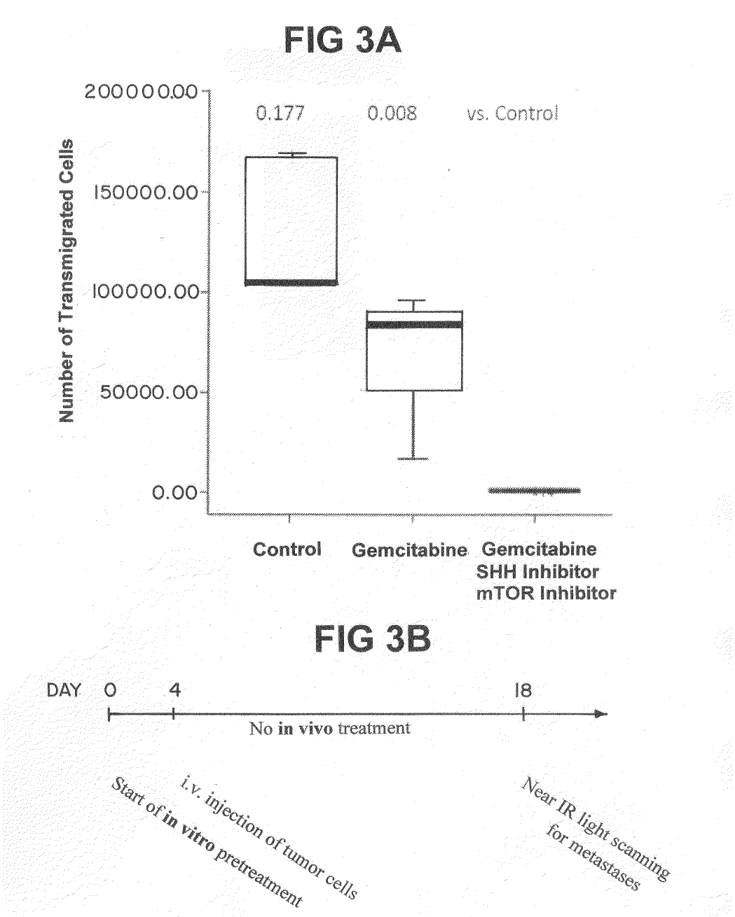

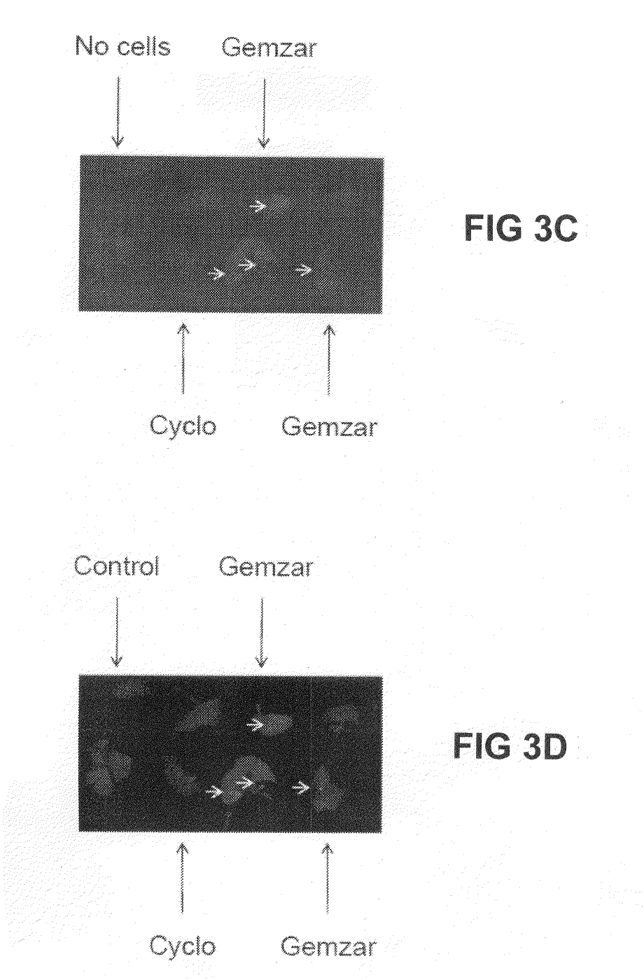

The transmigratory activity of cells is an important functional assay representing the invasive capacity of cells. The combined therapy using all three substances (100 ng/ml Gemcitabine, 100 ng/ml Rapamycin, 10 μM Cyclopamine) drastically reduced the invasive capacity in this in vitro assay opposed to control or Gemcitabine alone (FIGS. 3A-3D). The validity of the assay was assessed by in vivo investigation of the metastatic activity of the treated cells following in vitro pretreatment (3C and 3D; white arrows indicate metastatic lesions).

### Example 1.3

### In-Vitro: Content of CSC by Flow Cytometry in Primary Tissue

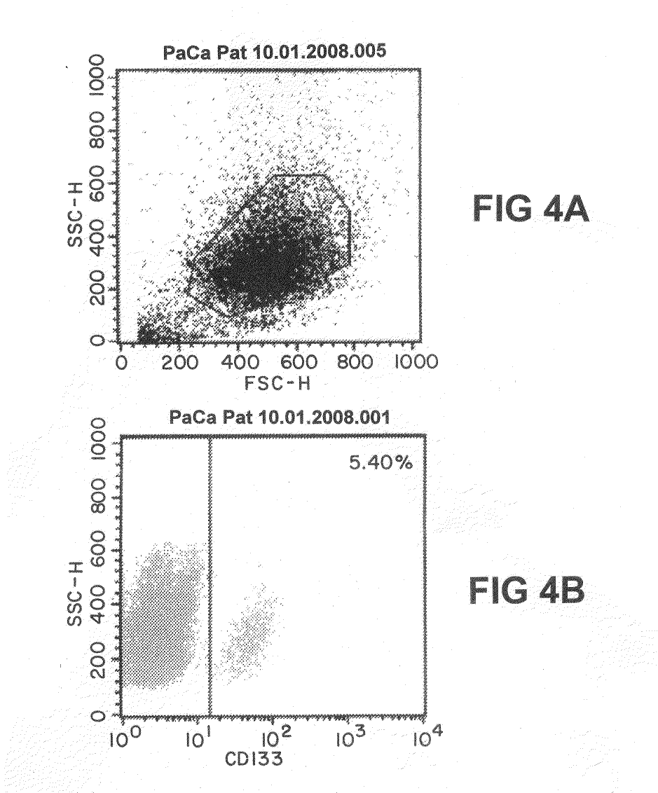

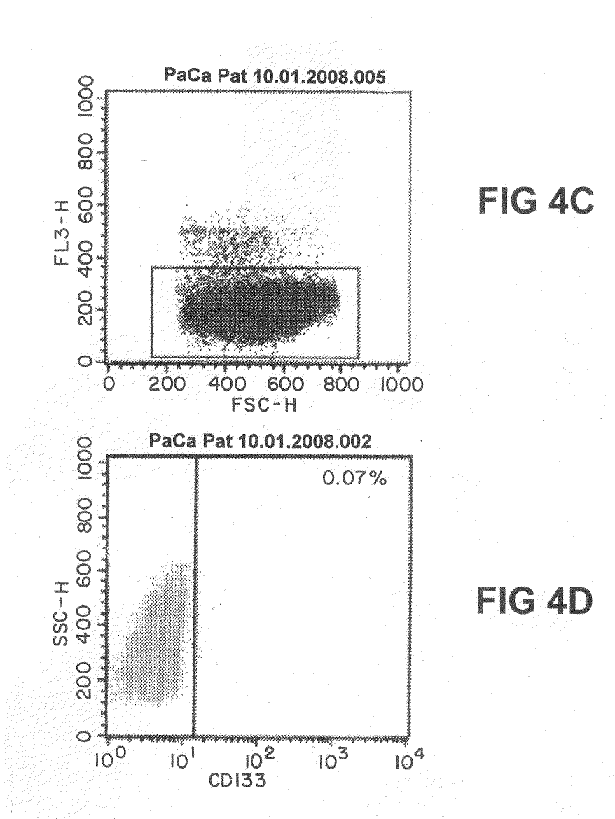

The findings from Examples 1.1 and 1.2 in tumour cell lines were reproduced in primary patient tissue with viability tested with propidium iodide (FIGS. 4A-4D). In vitro treatment with the combined therapy of 100 ng/ml Gemcitabine, 100 ng/ml Rapamycin, and 10 μM Cyclopamine led to the elimination of CD 133⁺ cancer stem cells.

### Example 1.4

### In-Vivo: Tumorigenicity of Tumour Cells After Pretreatment

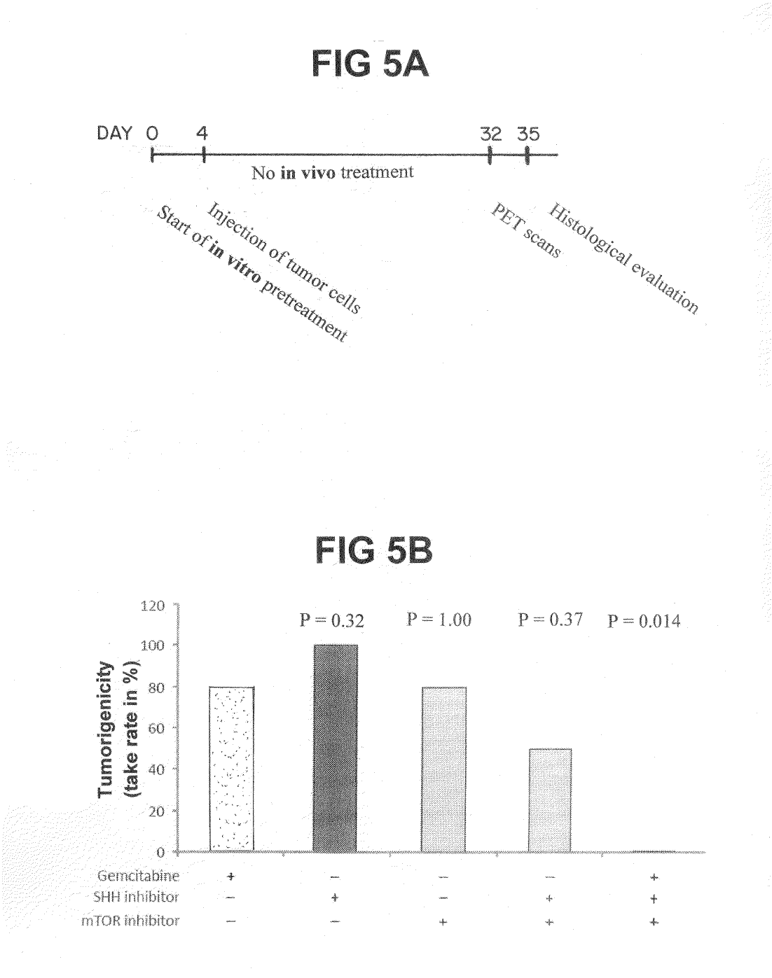

Following a common practice for the definition of cancer stem cells a further experiment was done to demonstrate any potential loss of tumorigenicity after transplantation of cells pretreated with different sets of treatments in vitro. In vitro pre-treatment using the combination of all three substances (100 ng/ml Gemcitabine, 100 ng/ml Rapamycin, and 10 μM Cyclopamine) led to a complete reversal of tumorigenicity (FIGS. 5A-5B) opposed to control or separate treatment with the substances of the active substance combination or even with a double combination of Rapamycin and Cyclopamine. Cultivated pancreatic cancer cells were exposed to either Gemcitabine alone (representing clinical standard therapy), one of the stem cell pathway inhibitors alone, combination of inhibitors, or a combination of Cyclopamine, Rapamycin, and Gemcitabine in the above given respective concentrations. After 96 hours of pretreatment, the remaining viable cells were orthotopically implanted into the pancreas of mice, which received no further treatment in vivo.

### Example 1.5

### In-Vivo: Tumorigenicity of Tumour Cells After Pretreatment

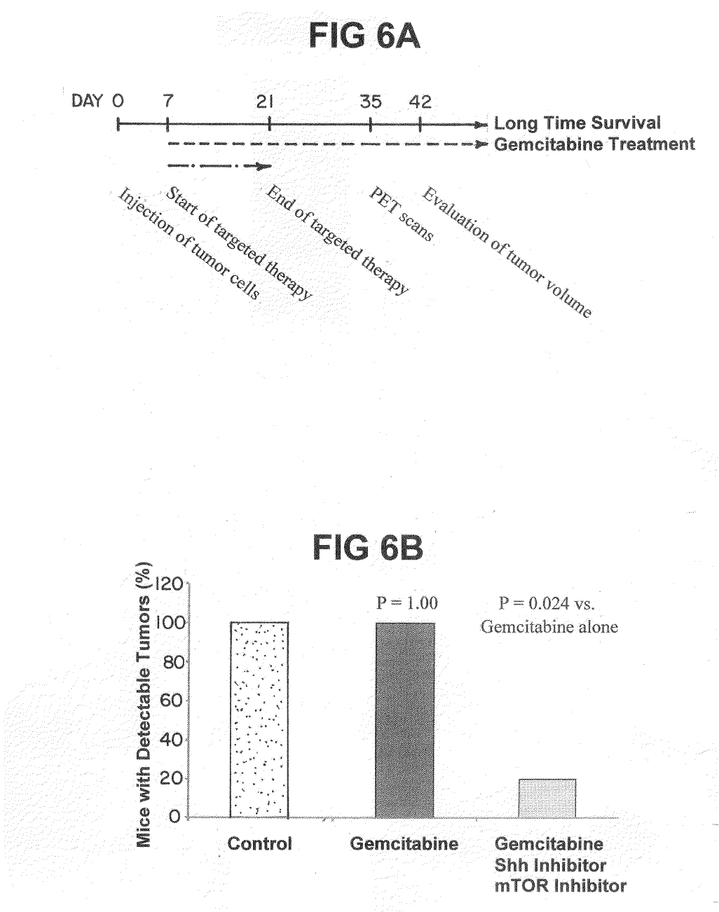

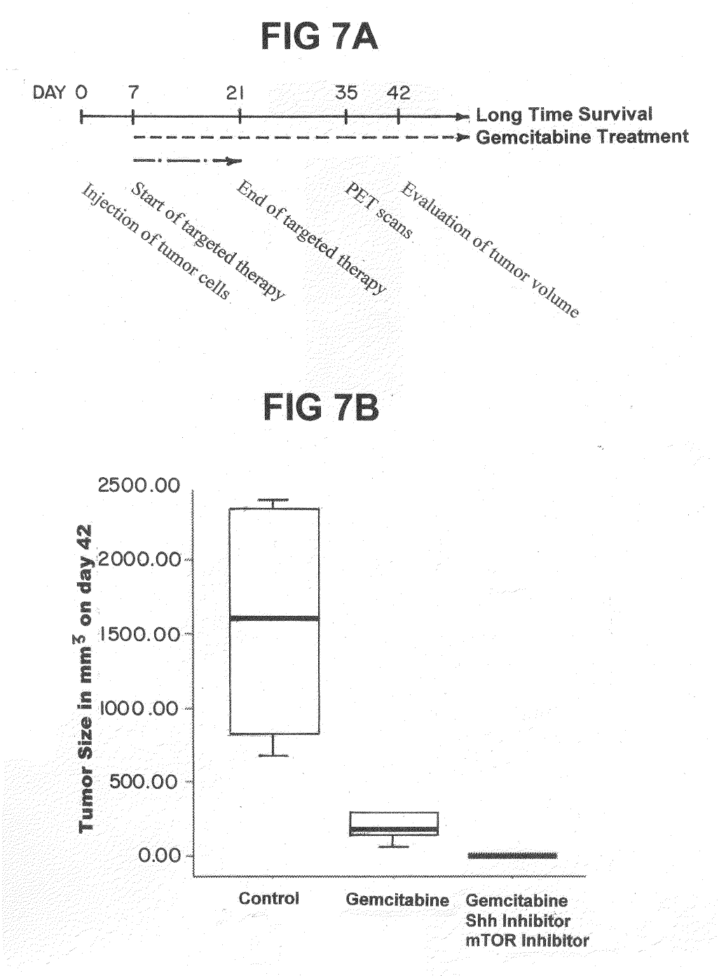

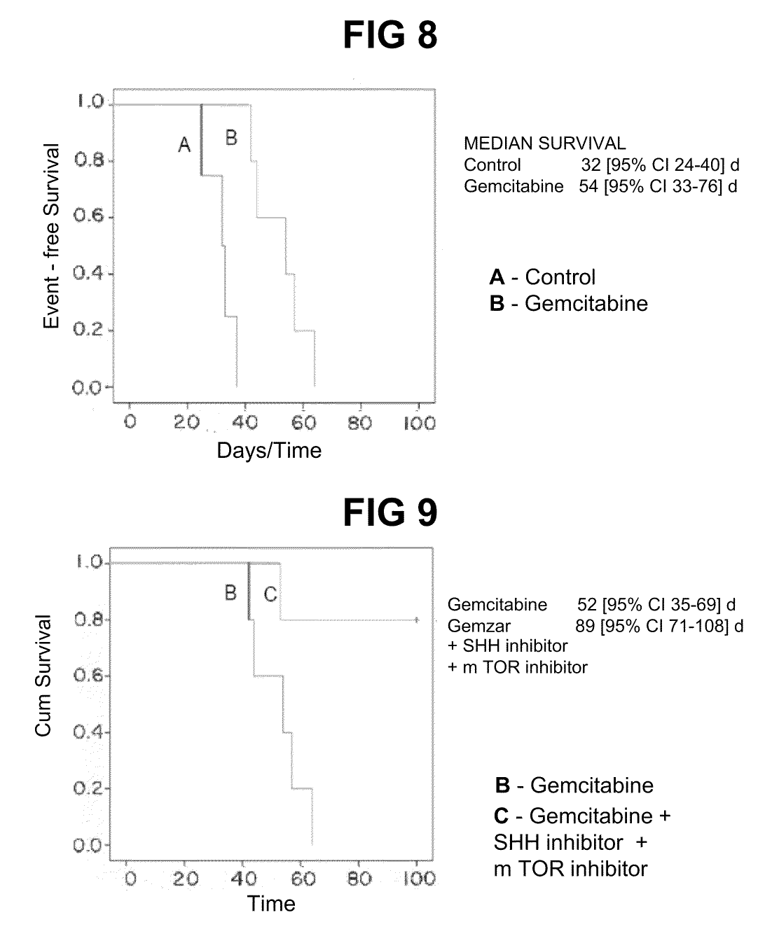

One week after cell implantation, successful tumour implantation was confirmed and tumour-bearing mice were randomized into three groups receiving either no treatment, or Gemcitabine alone, or in combination with Cyclopamine and Rapamycin. Gemcitabine was administered twice a week by intraperitoneal injections at 125 mg/kg BW. Cyclopamine was used as 25 mg/kg by oral gavages twice daily and Rapamycin was orally administered via the drinking water (5 mg/kg). Mice were treated with Gemcitabine for the entire duration of the study and simultaneously received Cyclopamine and Rapamycin for 14 days. No palpable tumour could be found in 80% of mice treated with the triple combination mentioned above (FIGS. 6A-6B) and tumour size was significantly smaller compared to control (FIGS. 7A-7B) Importantly, while standard therapy with Gemcitabine resulted in a prolongation of median survival by 22 days (FIG. 8), combination treatment translated into a prolonged tumour- and metastasis-free survival of animals compared to control (FIG. 9). Intriguingly, the majority of the animals survived the extended follow-up period of 100 days.

### Example 2

### Double Active Substance Combination of Gemcitabine and the Nodal/Activin Inhibitor SB431542

### Example 2.1

### In-Vitro: Content of CSC by Flow Cytometry in Tumour Cell Lines

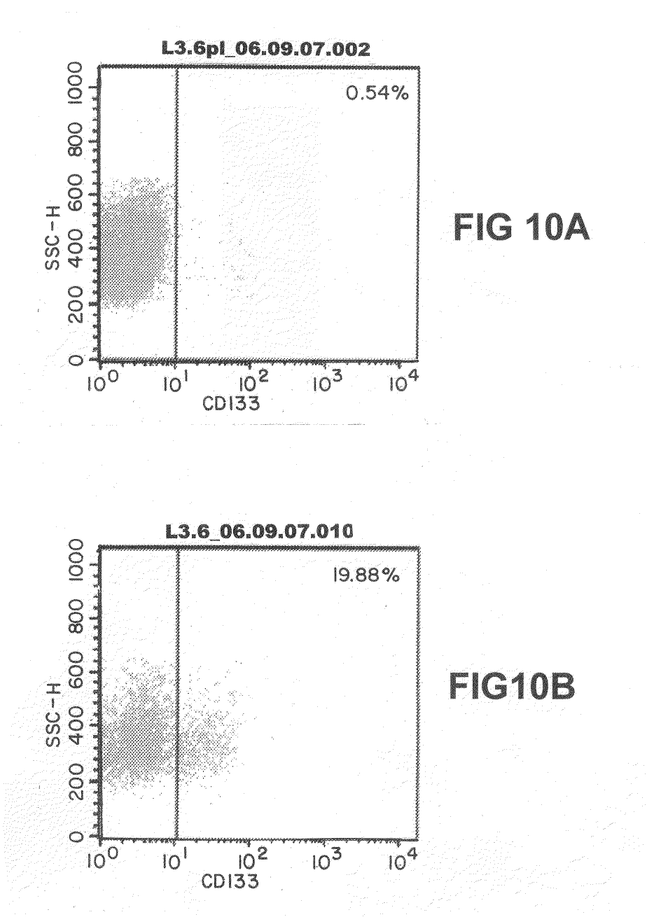

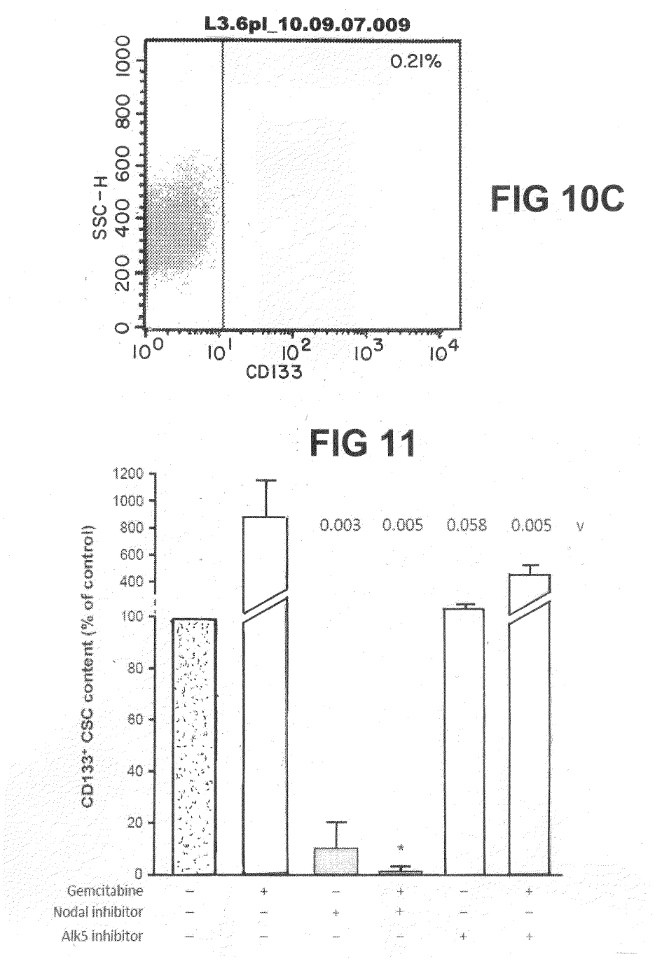

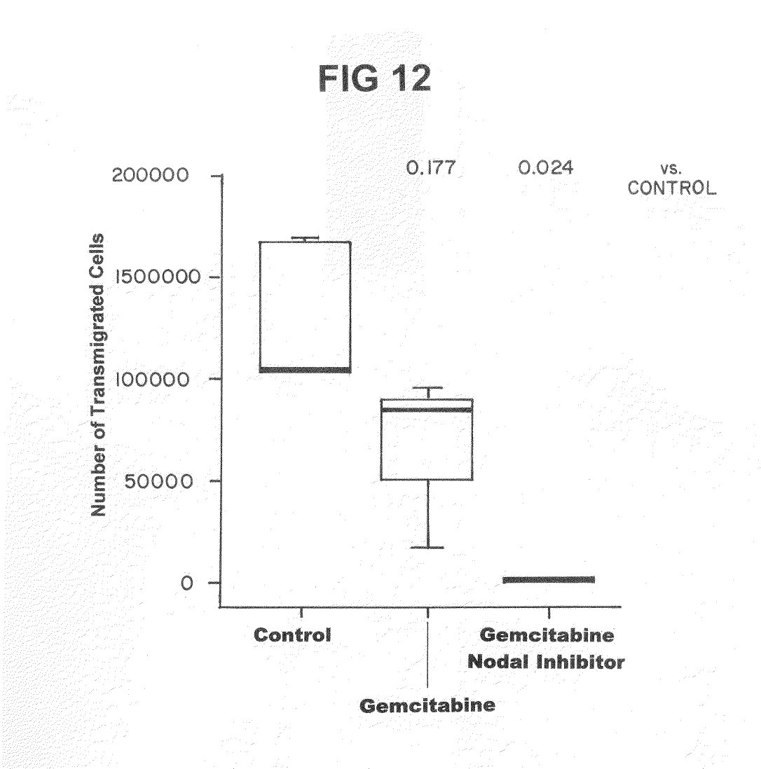

As a first step the content of cancer stem cells (measured as “CD 133-content”) within the entire tumour cell population was measured by flow cytometry (FIGS. 10A-10C). Separate treatment with 5 μM SB431542 alone (the sole blockade of the Nodal/Activin signalling pathway as monotherapy) already resulted in a significant reduction in CD133⁺ cancer stem cells in our experiments (FIG. 11), whereas single treatment with 100 ng/ml Gemcitabine alone did not. With treatment with the active substance combination of 100 ng/ml Gemcitabine and 5 μM SB431542 a complete elimination of CD133⁺ cancer stem cells could be achieved in vitro. Additionally, the transmigratory activity as an important functional marker for the invasiveness of these cells was almost completely inhibited after combination therapy with this double combination of Gemcitabine and SB431542 (FIG. 12) opposed to control or single treatment with Gemcitabine.

### Example 2.2

### In-Vitro: Content of CSC by Flow Cytometry in Primary Tissue

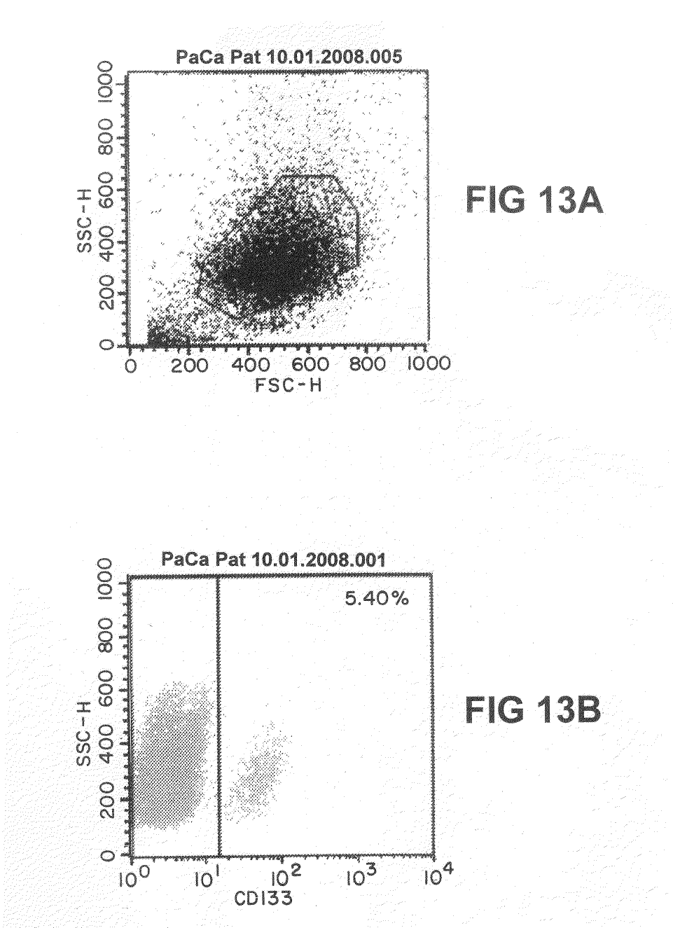

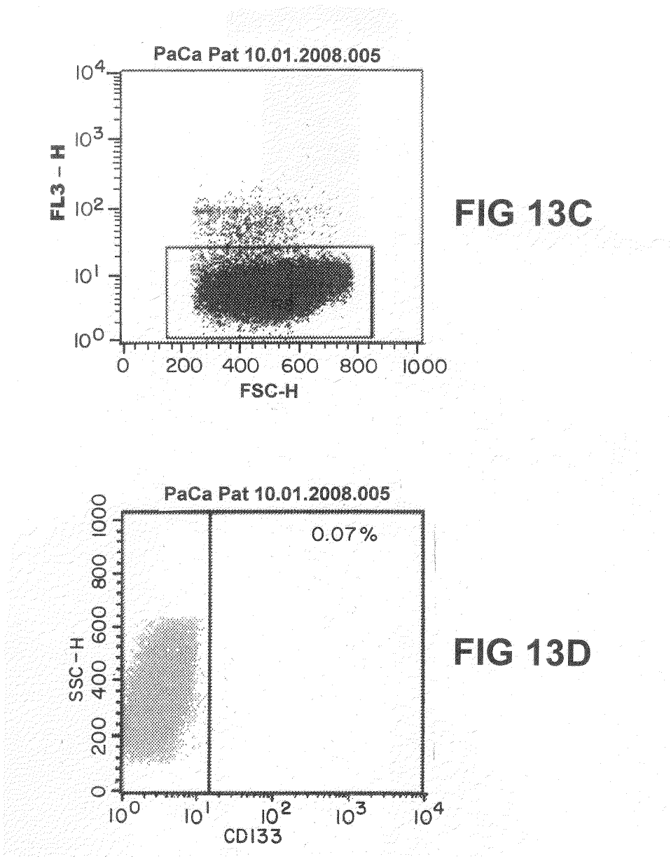

The findings from Examples 2.1 in tumour cell lines were reproduced in fresh primary tumour cells from patients with pancreatic cancer with viability tested with propidium iodide (FIGS. 13A-13D). In vitro treatment with the combined therapy of 100 ng/ml Gemcitabine and 5 μM SB431542 led to the elimination of CD 133⁺ cancer stem cells.

### Example 23

### In-Vivo: Tumorigenicity of Tumour Cells After Pretreatment

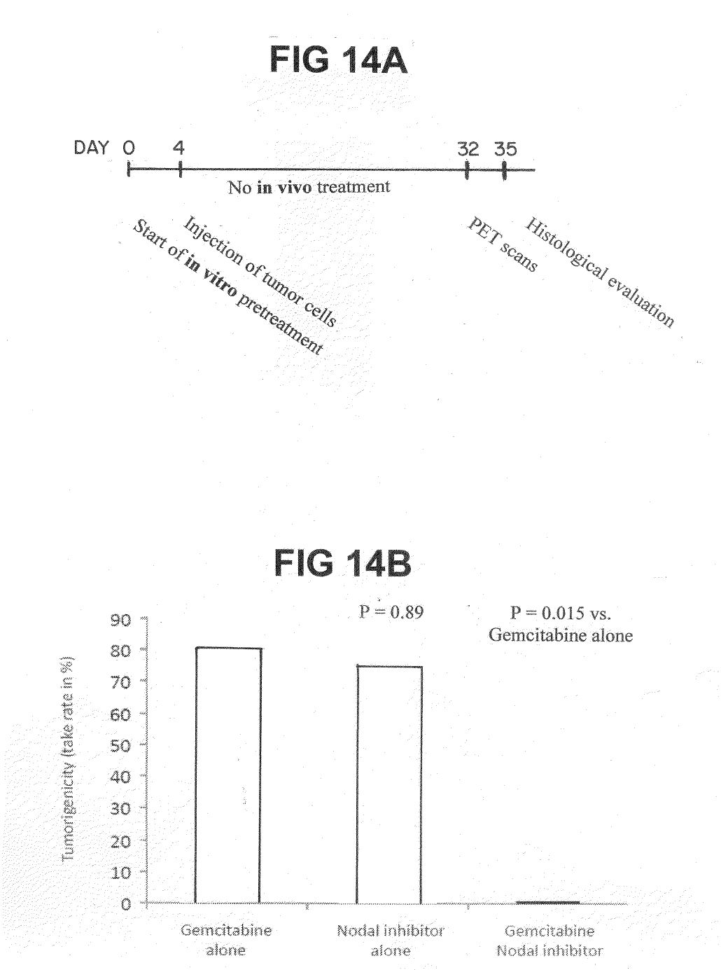

Following a common practice for the definition of cancer stem cells a further experiment was performed to demonstrate any potential loss of tumorigenicity after transplantation of cells pretreated with different sets of treatments in vitro in an orthotopic mouse model of pancreatic cancer. In-vitro pre-treatment with either 100 ng/ml Gemcitabine, or 5 μM SB431542 alone or the double active substance combination of Gemcitabine and SB431542 led to a complete reversal of tumorigenicity with the double combination and close to no effect of the substances alone (FIGS. 14A-14B). Cultivated pancreatic cancer cells were exposed to either Gemcitabine alone representing clinical standard therapy, SB431542 alone, or a combination of Gemcitabine and SB431542 in the above given respective concentrations. After 96 hours of pretreatment, the remaining viable cells were orthotopically implanted into the pancreas of mice, which received no further treatment in vivo.

### Example 2.4

### In-Vivo: Tumorigenicity of Tumour Cells

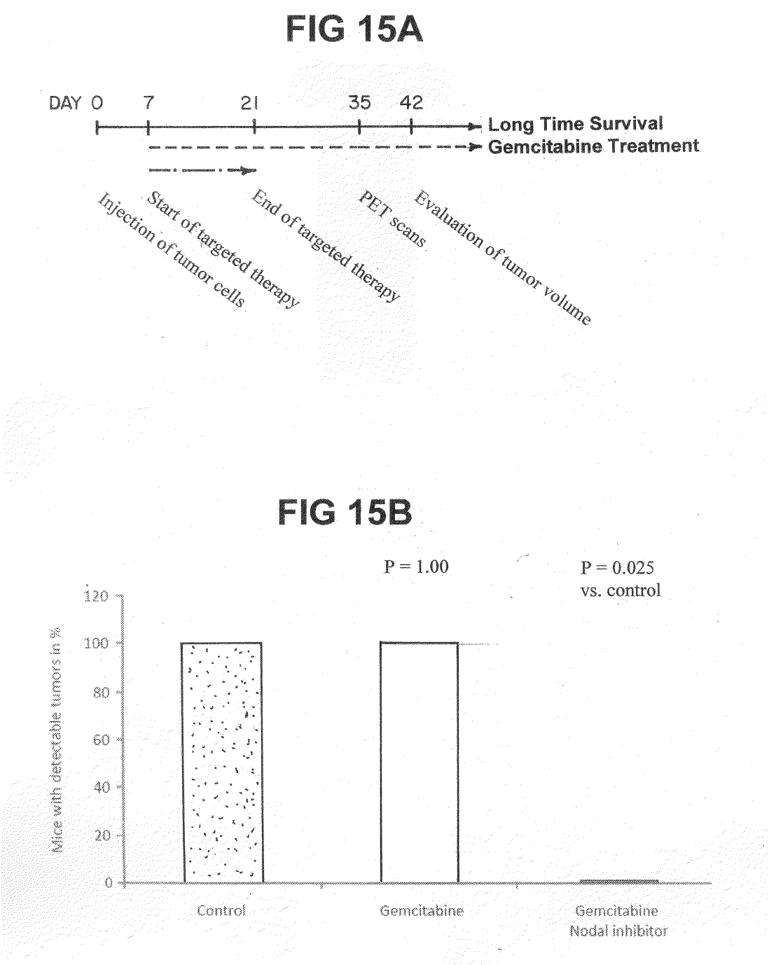

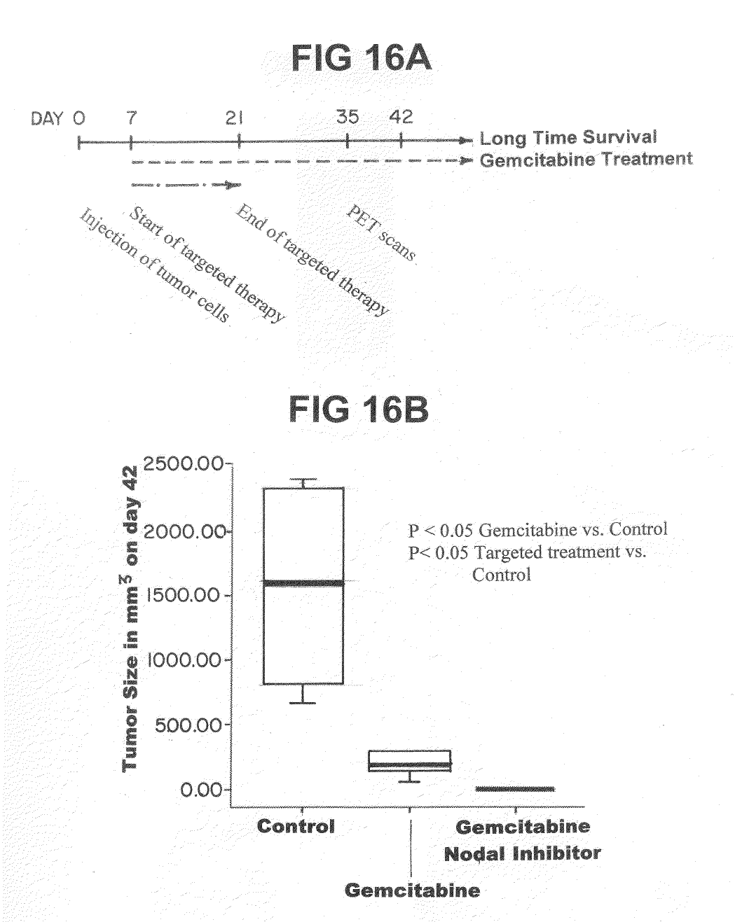

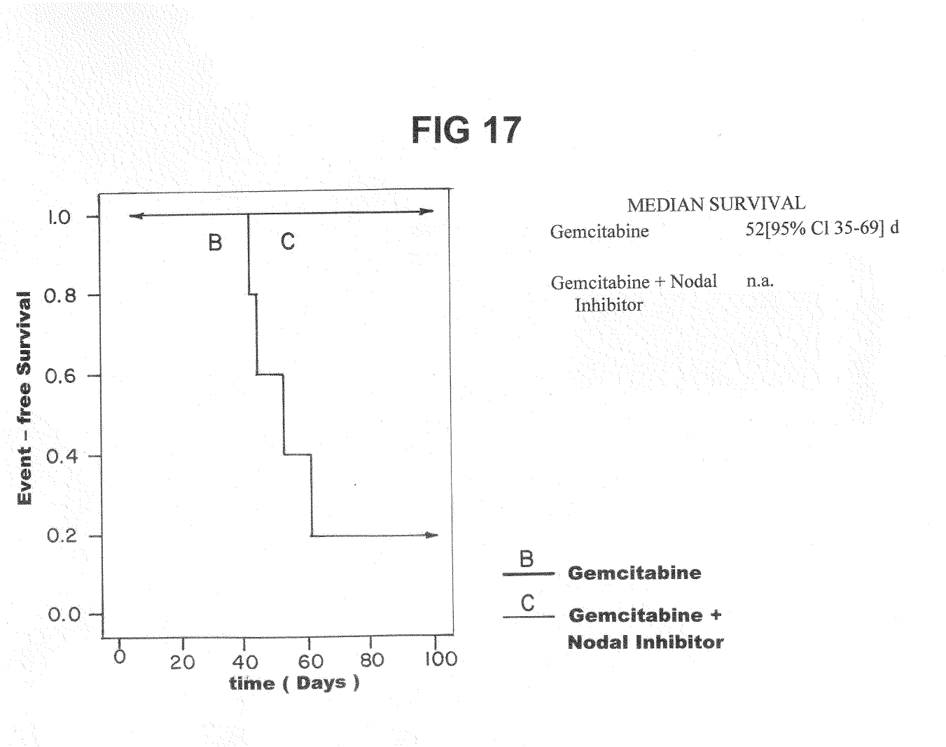

Seven days after orthotopic implantation of tumour cells, therapy with either Gemcitabine alone or in combination with targeted Nodal/Activin inhibition by SB431542 was initiated. The double combination therapy was stopped after 14 days of treatment, the single Gemcitabine treatment was continued until sacrifice of the animals. 35 days after tumour cell treatment, PET scans were done and evaluation of tumour volume was performed on day 42. None of the investigated mice treated with combination therapy showed evidence for tumour formation (FIGS. 15A-15B). Contrarily, tumour incidence was unaffected by Gemcitabine monotherapy, which merely resulted in reduction of tumour size (FIGS. 16A-16B). The long-time tumour- and metastasis-free survival of the animals receiving combination therapy was striking as compared to Gemcitabine alone treated mice (FIG. 17).

### Example 3

### Detailed Experiments Using Double Active and Triple Active Substance Combinations

### Example 3.1

### In Vitro Evaluation of Anti-Cancer Stem Cell Agents

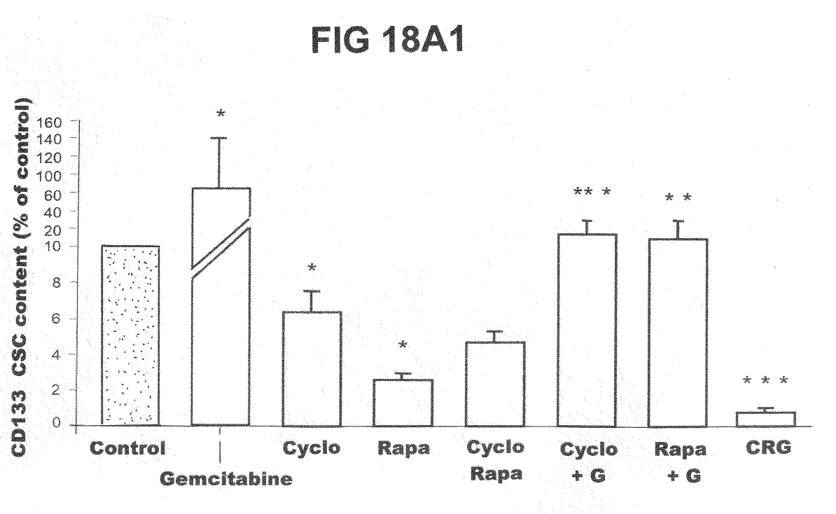

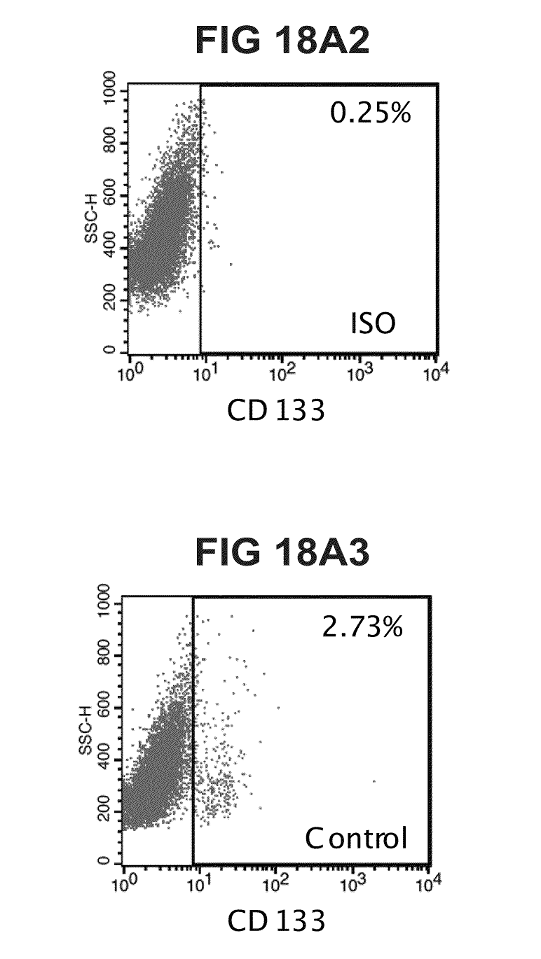

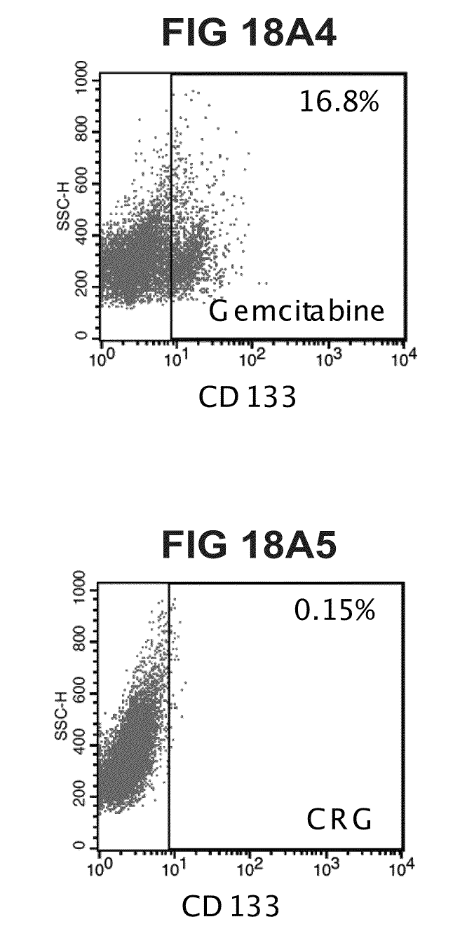

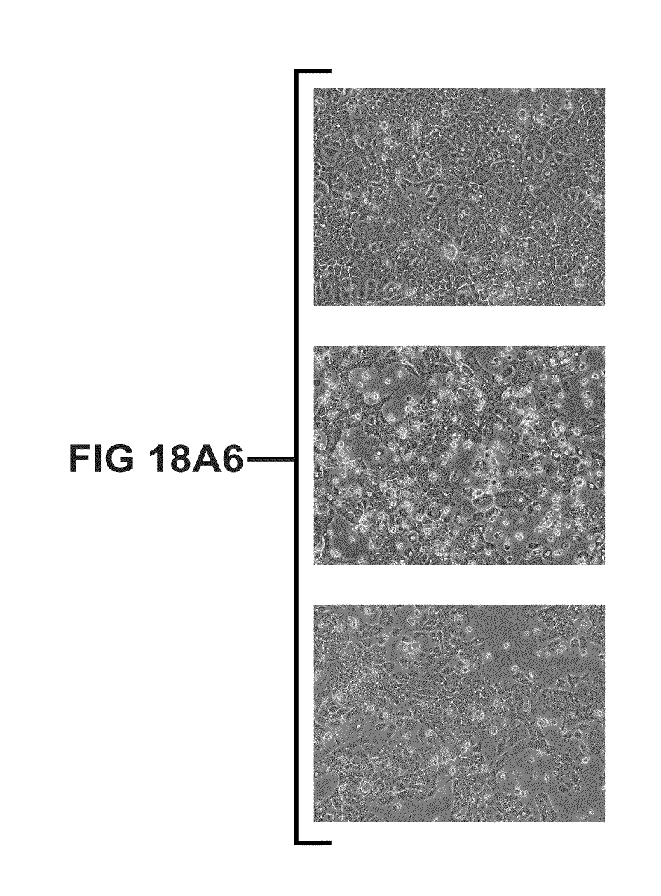

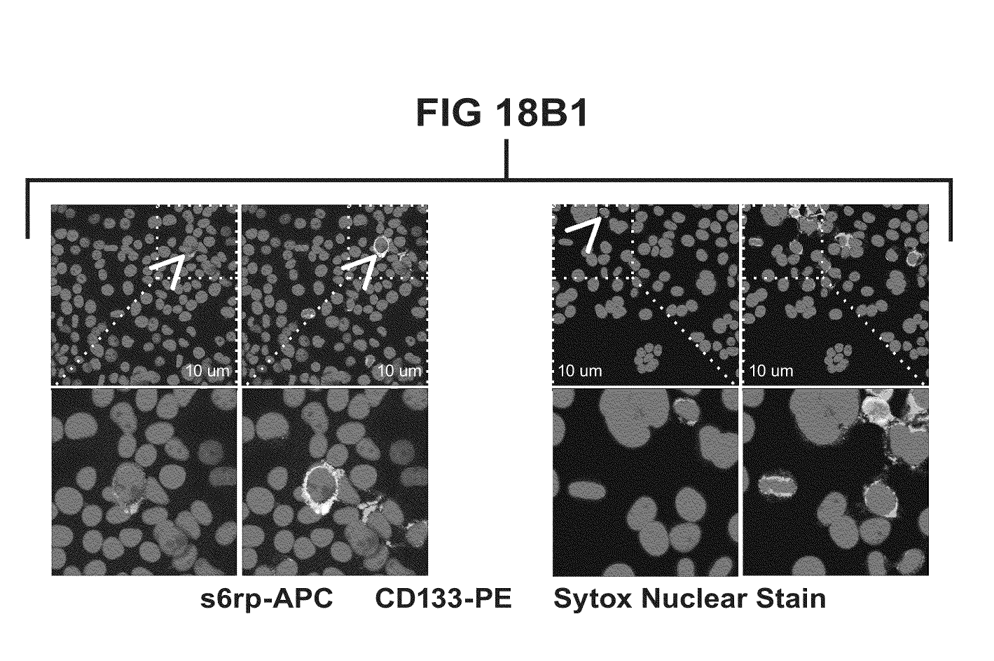

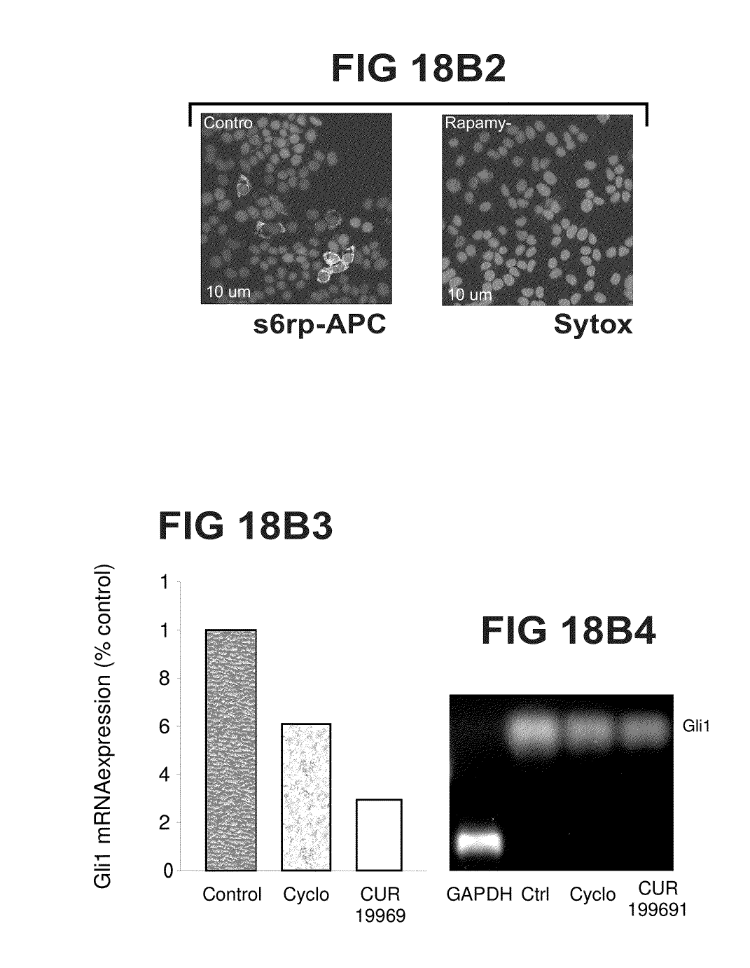

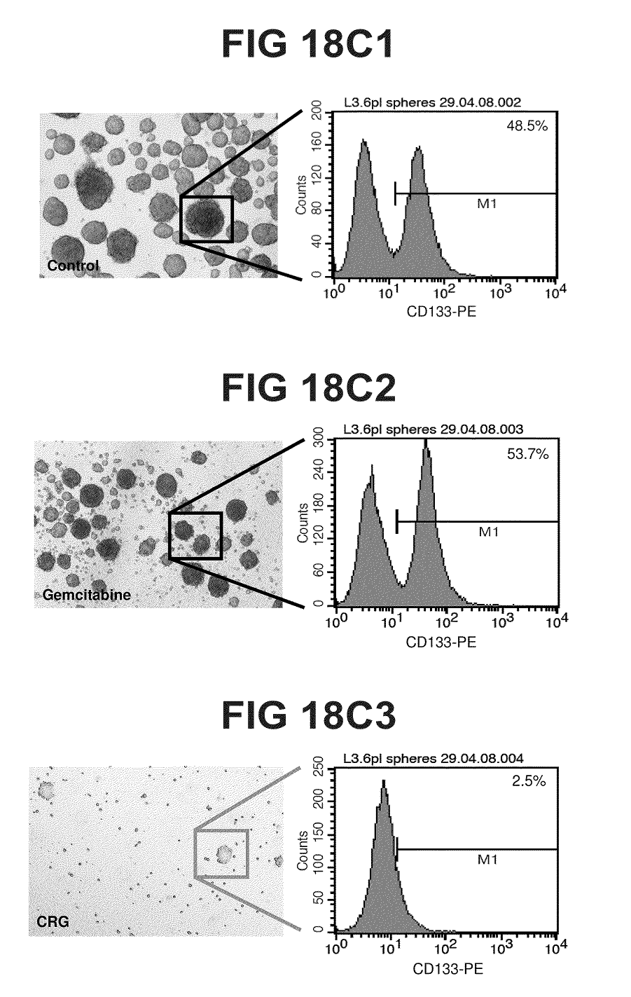

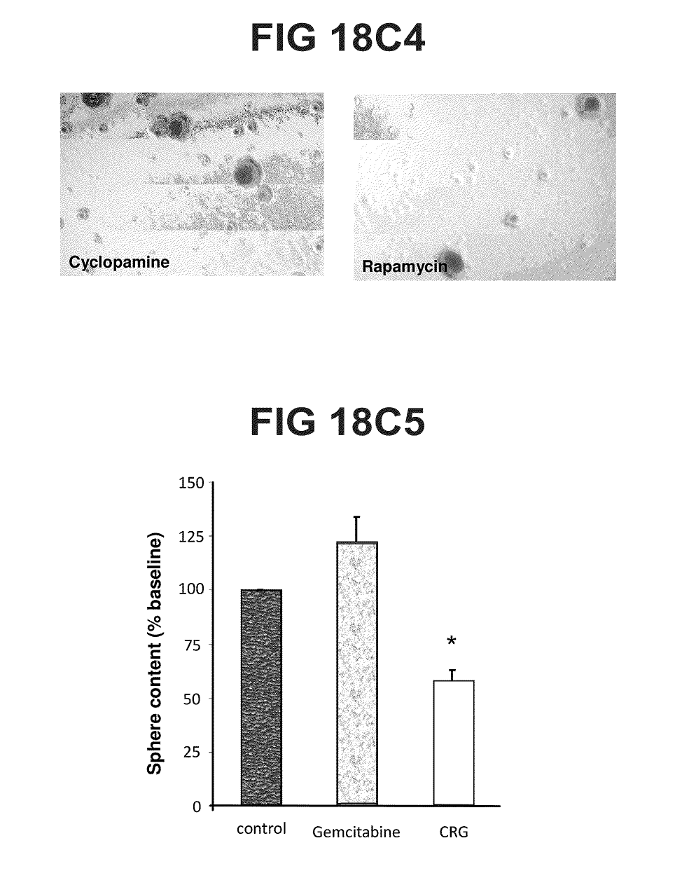

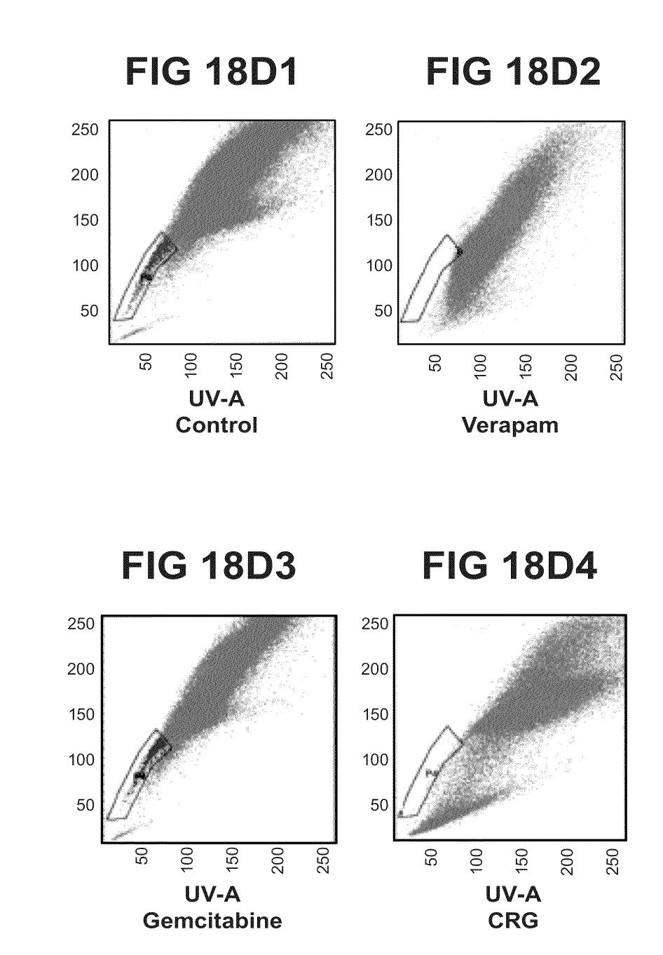

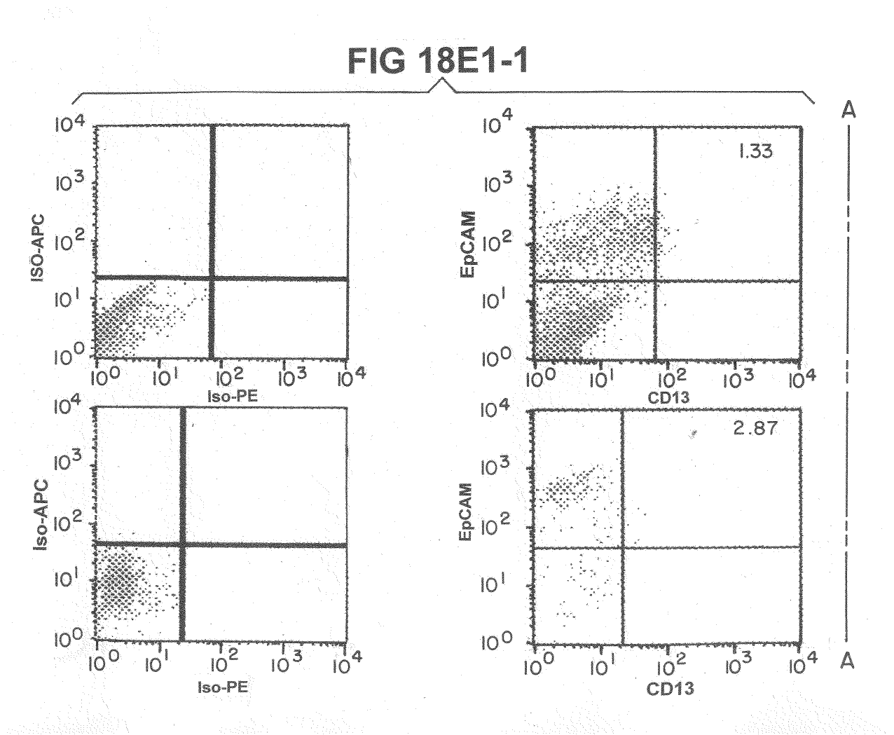

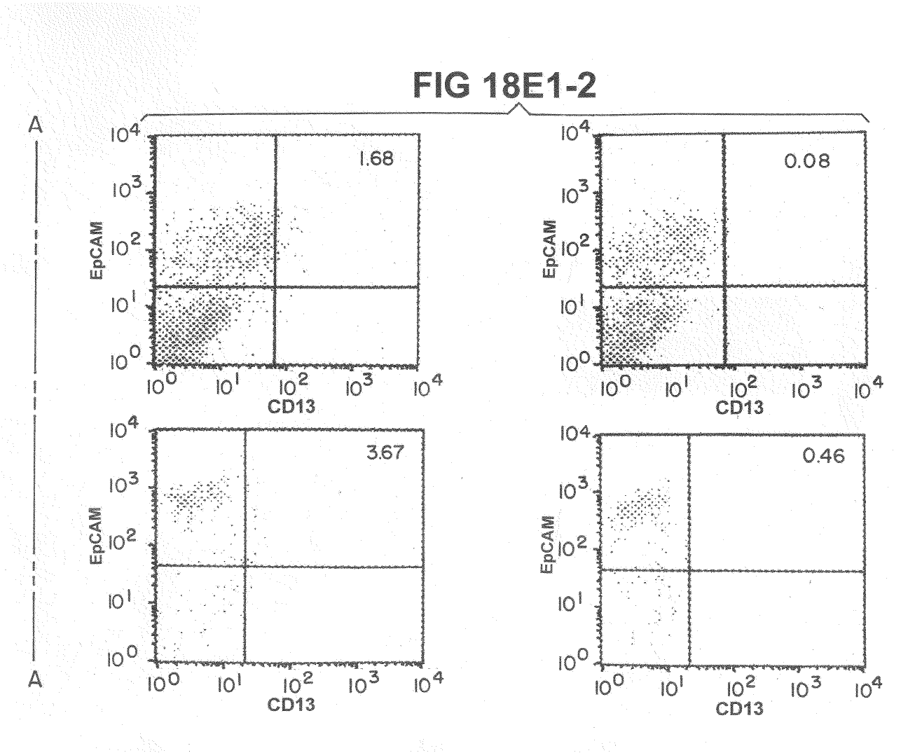

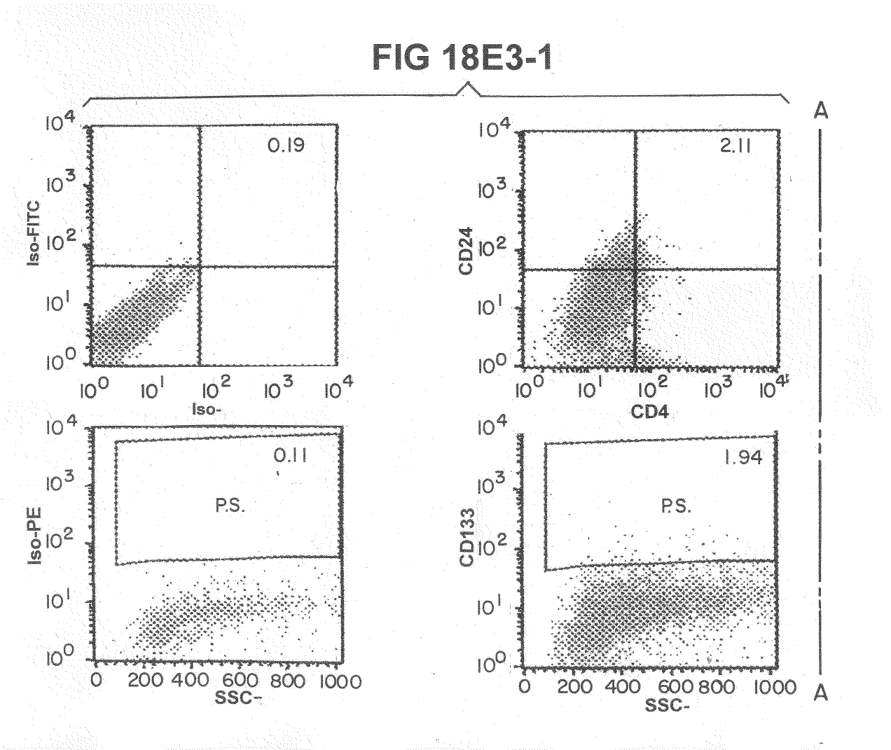

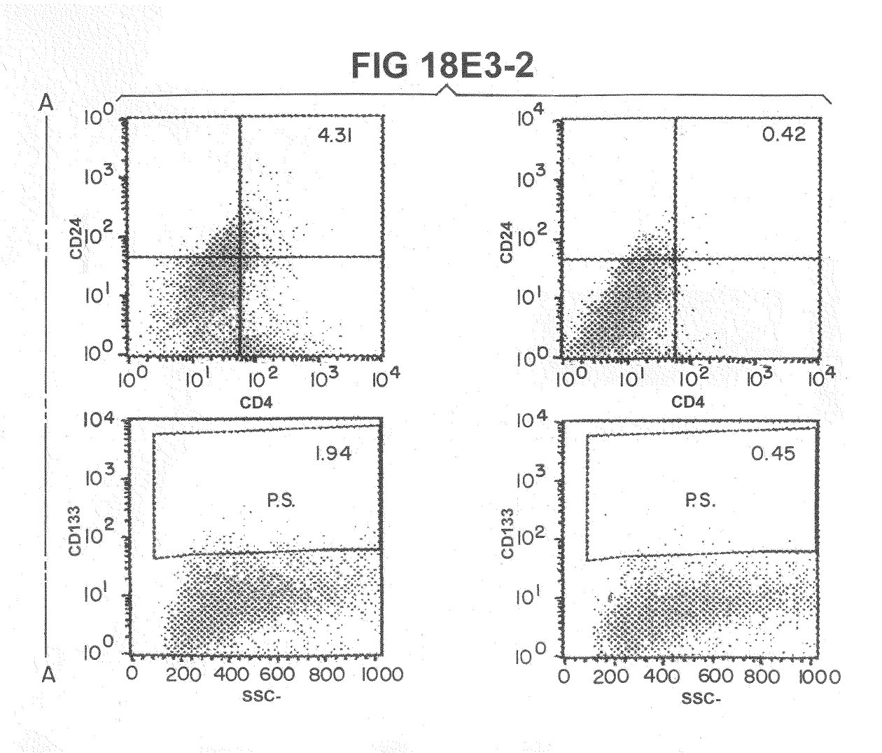

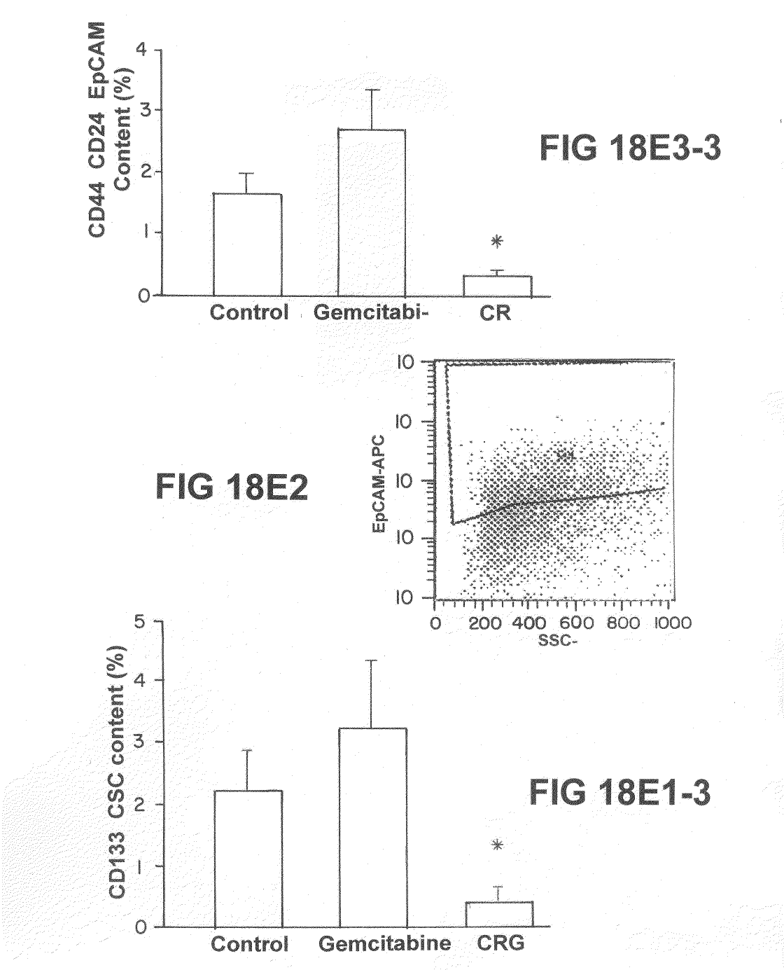

In this experiment, potentially effective substances for the targeted elimination of CD133⁺ pancreatic cancer stem cells (CSC) were screened by means of flow cytometry following 48 hours of therapy. Consistent with the previous reports, a marked enrichment of CD133⁺ cells following Gemcitabine therapy was observed (FIGS. 18A1-18A6). As a Gemcitabine concentration of 100 ng/ml was the lowest concentration capable of achieving a strong relative enrichment for CD133⁺ cells through depletion of CD133⁻ cells, this concentration was chosen for subsequent experiments. A single-agent therapy with the SHH inhibitor Cyclopamine and the mTOR inhibitor Rapamycin already resulted in a moderate, albeit significant decrease in the CD133⁺ cell content (FIG. 18A1).

To assess whether mTOR signalling is indeed active in CSC histological analyses for the phosphorylation of p70s6-kinase were performed, a downstream target of mTOR that has been shown to be a reliable marker for the activity of the mTOR-pathway. Interestingly, mTOR signalling was active only in a small subset of cells including CD133⁺ CSC (FIG. 18B1; upper panel). Following mTOR inhibition by Rapamycin, phosphorylation of p-70-s6-kinase in CSC was profoundly reduced (FIG. 18B2; lower panel).

The results for Cyclopamine were also well in line with data for aldehyde dehydrogenase as a CSC marker demonstrating a reduction to ⅓ of the original CSC population following Cyclopamine treatment. Consistently, a 40% decrease in hedgehog activation (FIGS. 18B3-18B4) was demonstrated here. Reasoning whether a stronger and more specific inhibition of hedgehog signalling by the small molecule inhibitor CUR199691 alone (60% reduction in Gli expression; 10 μM; Genentech, South San Francisco, Calif.) might be more potent in eliminating the CSC population, the inventors surprisingly did not achieve any further inhibition of the CSC content (55% CD133 content for Cyclopamine alone versus 56% for CUR199691 alone).

Therefore, since a considerable percentage of CSC remained detectable even for the combination of the inhibitors with Gemcitabine, the efficacy of a combined treatment regimen (Cyclopamine, Rapamycin, and Gemcitabine; CRG) was then evaluated. Intriguingly, CRG treatment virtually eliminated all CD133⁺ CSC (FIGS. 18A2-18A5). Interestingly, a marked difference in the gross number of floating cells between Gemcitabine alone and CRG was not observed (FIG. 18A6). While Gemcitabine had a significant effect on the overall survival of cells, CRG did not seem to have an additional effect on the general cell population. Consistently, investigation of the pancreatic cancer cell line AsPC providing the opportunity to evaluate their cancer stem cell population using a different set of surface antigens, namely CD24 and CD44, provided similar results. To further extend these in vitro data, other techniques were also utilized that have previously been used for the identification of subpopulation of cells enriched for CSC. First, pancreatic CSC were clonally expanded as CSC-enriched spheres (FIGS. 18C1-18C5). Floating spheres were treated in ultra low adhesion 6-well plates. After completion of the treatment, five randomly selected high-power fields were analyzed. Gemcitabine single-agent therapy resulted in a marked relative increase of CD133⁺ cells, consistent with a marked chemo-resistance of CD133⁺ cells while Cyclopamine or Rapamycin alone resulted in the reduction of tumour spheres with the resulting single cells eventually dying when kept in these specific stem cell conditions. Most importantly, CRG combination therapy demonstrated the strongest potential for tumorsphere depletion. Moreover, side population (SP) cells were unaffected by exposure to Gemcitabine whereas CRG triple therapy virtually depleted all SP cells (FIGS. 18D1-18D4).

### Example 3.2

### Treatment Effects on Primary Pancreatic Cancer Cells

The above experiments were performed using an established human pancreatic cancer cell line, which was previously demonstrated to be an excellent model of pancreatic stem cells. In order to more conclusively demonstrate the clinical significance of the above findings, additional experiments were performed with freshly isolated primary pancreatic cancer cells. Consistent with the treatment effects observed for L3.6pl cells, Gemcitabine monotherapy tended to relatively enrich for the CSC fraction while combination therapy with CRG virtually abolished both CD133⁺ and CD24⁺CD44⁺EpCAM⁺ CSC populations, respectively (FIGS. 18E1-1-18E3-3). Of note, a heterogeneity with respect to treatment response was recorded: some patients showed a remarkably strong effect on the CSC content comparable to the effect observed in L3.6pl cells, others showed a more modest effect; an effect that correlated with the response to Gemcitabine as evidenced by the relative enrichment of the CSC fraction in the individual patient.

### Example 3.3

### Inhibition of SHH Eliminates Metastatic Activity

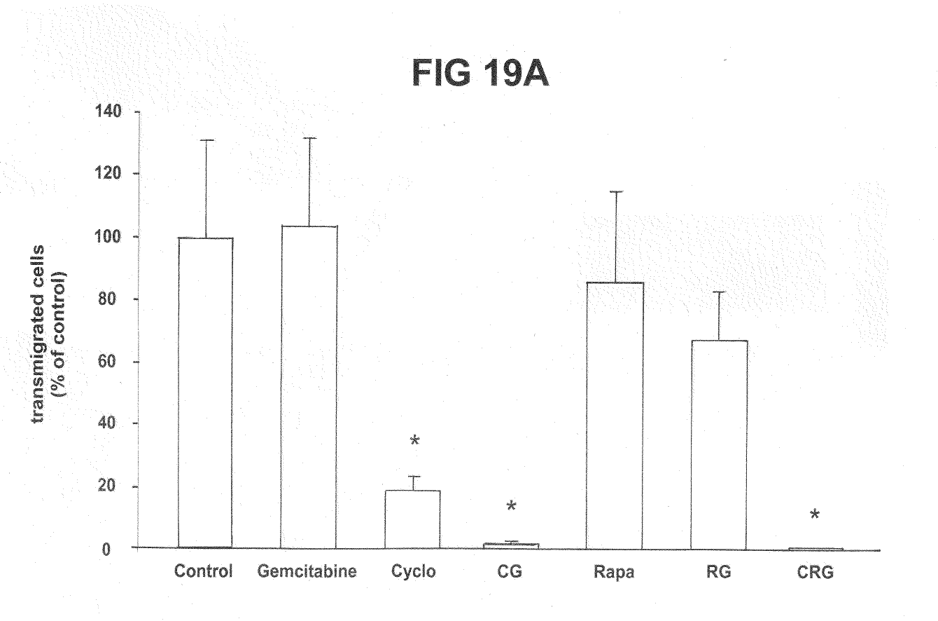

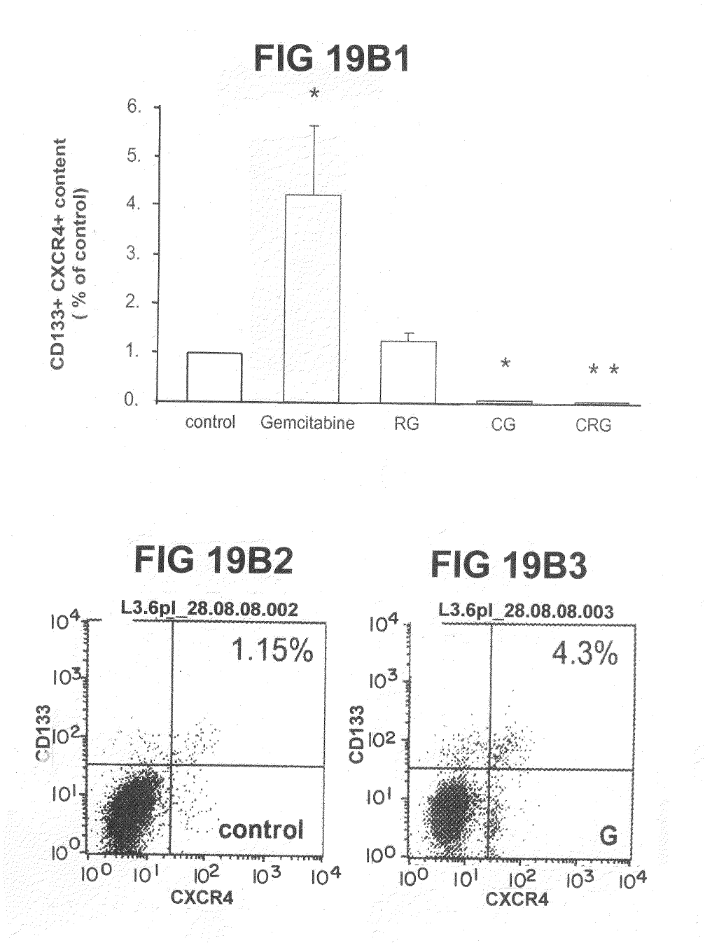

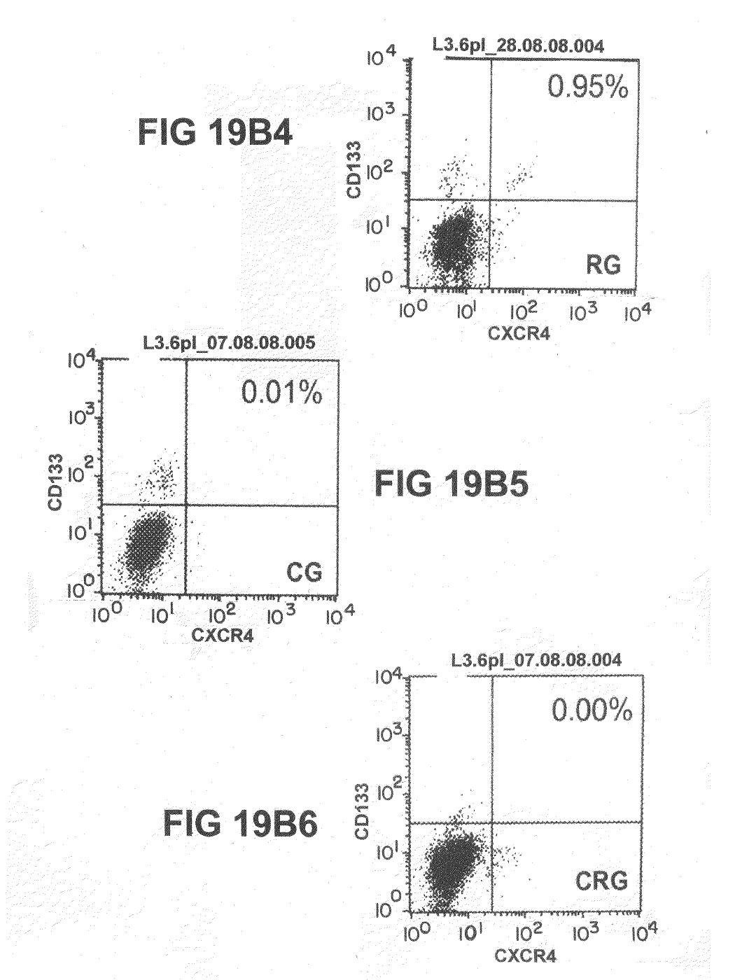

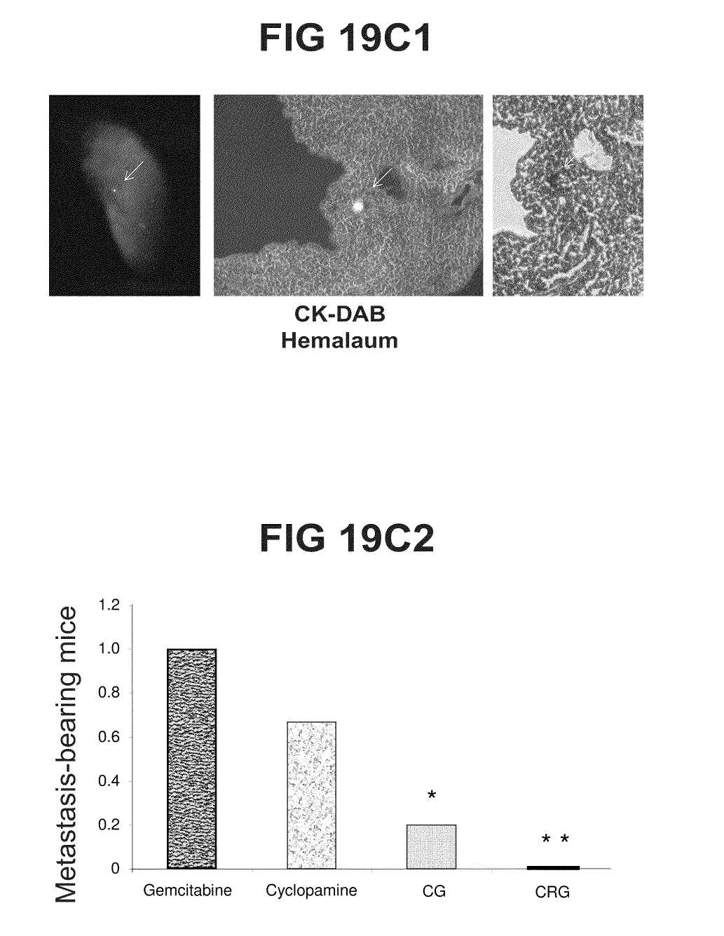

Recently, the inventors have shown that the chemokine receptor CXCR4 and its specific lig- and Stromal-Derived Factor-1 (SDF-1) plays a pivotal role in metastasis of pancreatic CSC (see Hermann et al., 2007, supra). In a modified Boyden chamber assay using SDF-1 as the migratory stimulus, Gemcitabine, Rapamycin, both alone, and their combination were unable to significantly reduce the migratory activity. In contrast, Cyclopamine alone already showed a strong reduction, but only combination with Gemcitabine resulted in complete abrogation of functional capacity in vitro (FIG. 19A). In line with these observations, flow cytometry demonstrated that treatment with Gemcitabine (middle panel) enriches for metastatic CSC, also named migrating CSC, which are characterized by co-expression of CD133 and CXCR4, whereas only CG (Cyclopamine, and Gemcitabine; CG) and CRG (Cyclopamine, Rapamycin, and Gemcitabine; CRG), respectively, resulted in a complete elimination of this CD133⁺ CXCR4⁺ CSC subpopulation, providing a rationale for further evaluating these treatment modalities with respect to their anti-metastatic effect in vivo (FIGS. 19B1-19B6). Pretreated and Qtracker-labeled cells were systemically infused. Seeded cells were tracked using near infrared scanning of the lungs as the primary target organ. Positive signals were confirmed by histological analysis. While all mice receiving Gemcitabine-pretreated cells showed evidence for metastasis, metastatic spread tended to be reduced in Cyclopamine-pretreated cells Importantly, combination of Gemcitabine and Cyclopamine further and significantly reduced metastatic activity while triple CRG therapy resulted in complete loss of metastatic activity in vivo (FIGS. 19C1-19C2).

### Example 3.4

### Loss of Tumorigenicity Following In Vitro Pre-Treatment

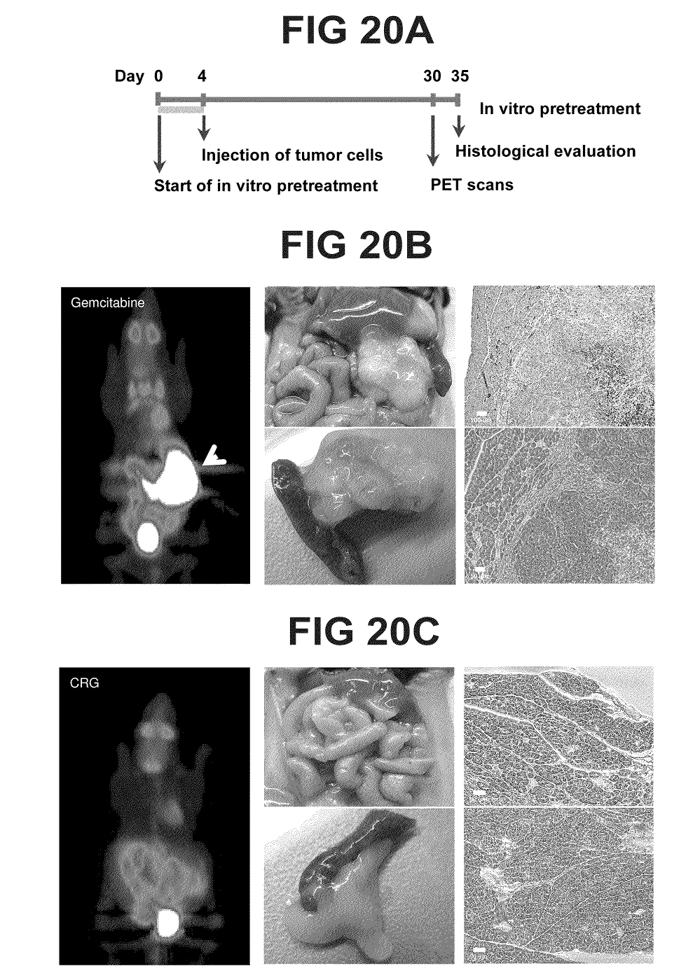

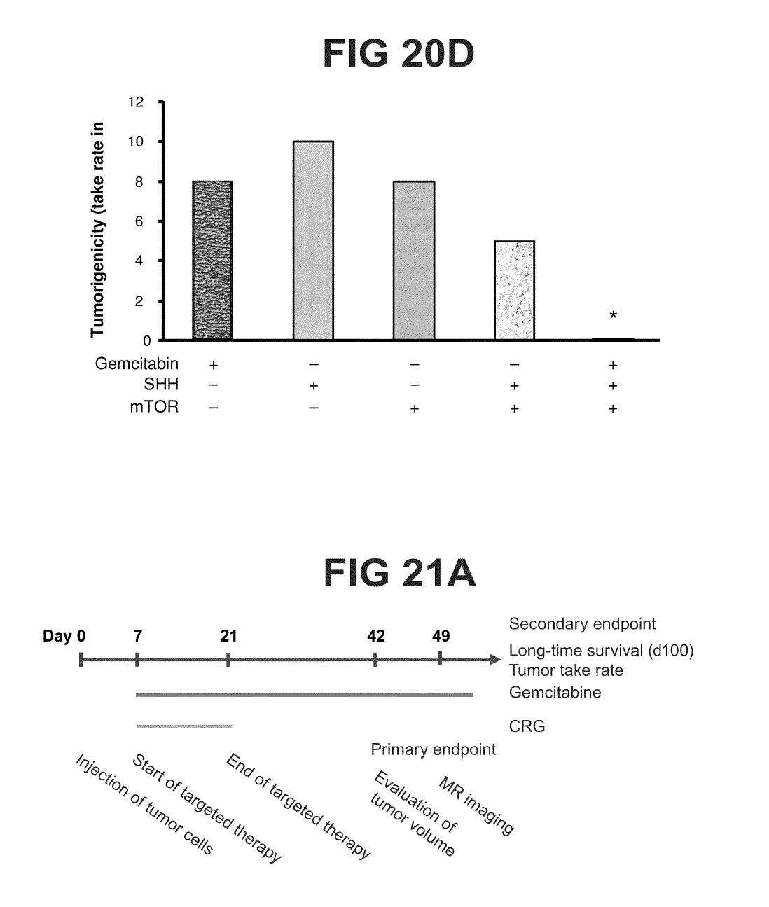

A prerequisite of CSC is their ability to form tumours in secondary recipients. Due to the limited number of cancer (stem) cells that can be obtained from primary tissues, it is virtually impossible to reproducibly perform in vivo treatment experiments with fresh patient-derived cells. Therefore, all subsequent in vivo experiments were performed with the established L3.6pl pancreatic cancer cells. The design of the experiment is illustrated in FIG. 20A. Based on above in vitro results, identical numbers of L3.6pl pancreatic cancer cells were exposed to either Gemcitabine alone, one of the stem cell pathway inhibitors alone, a combination of the inhibitors, or a combination of all three treatments. After 4 days of pretreatment, the surviving cells were orthotopically implanted into the pancreas, which received no further in vivo treatment. Tumorigenicity was first determined by PET scans on day 30 (FIGS. 20B, 20C; left panel) and by final macroscopic and microscopic histological evaluation on day 35 (FIGS. 20B, 20C; right panel). Surprisingly, neither Cyclopamine nor Rapamycin monotherapy significantly affected tumour take rates as compared to Gemcitabine (FIG. 20D). The combination therapy with Cyclopamine and Rapamycin showed a trend to a lower tumour take rate, which is consistent with above in vitro findings demonstrating no complete abrogation of the CSC population but a significant reduction and already a synergistic effect. The CRG combination therapy resulted in complete abrogation of tumorigenicity, clearly involving a synergistic effect.

### Example 3.5

### Targeted In Vivo Treatment Results in Enhanced Long-Term Tumour-Free Survival

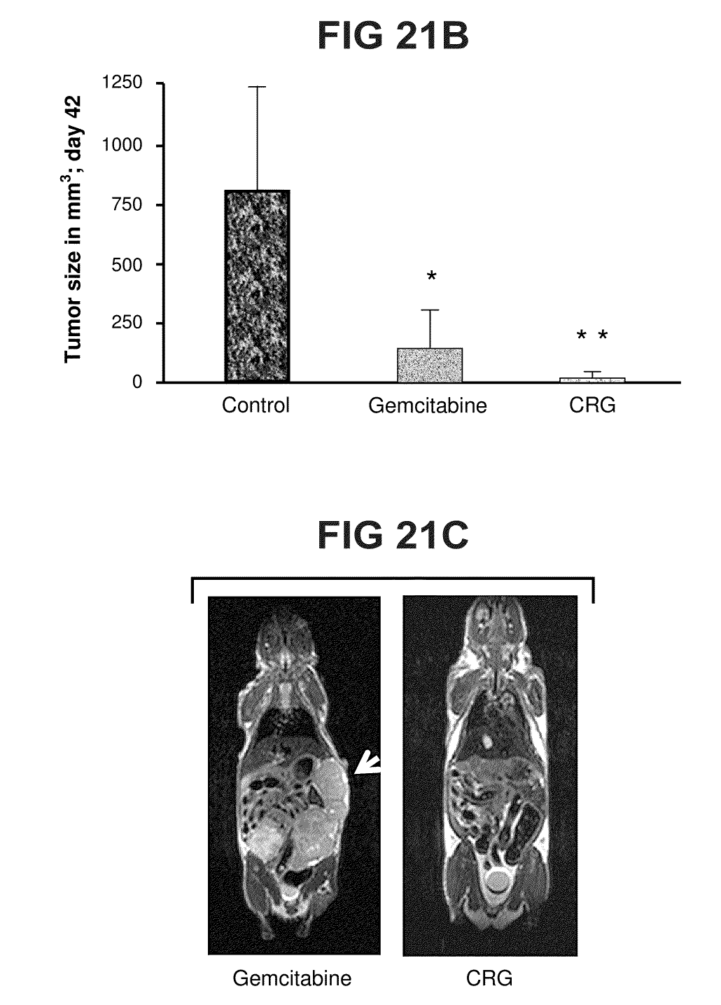

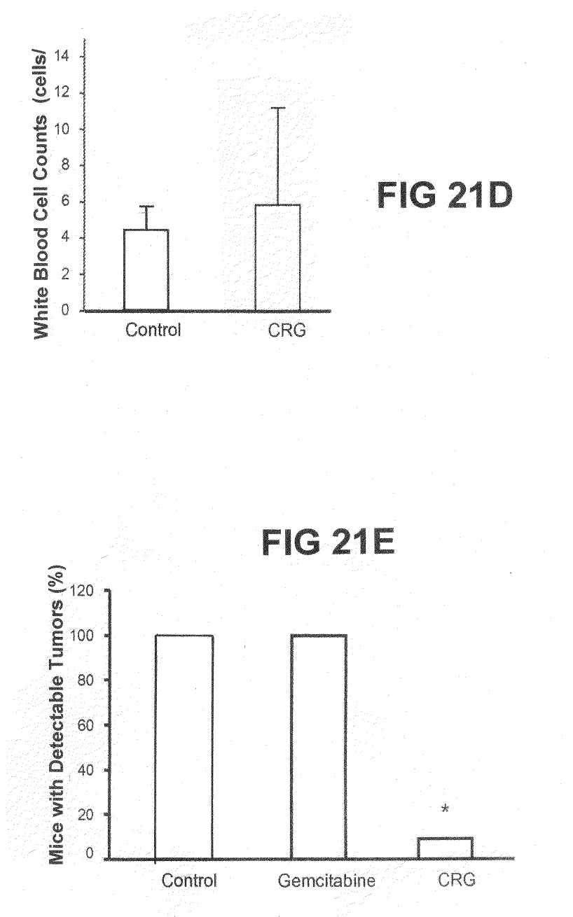

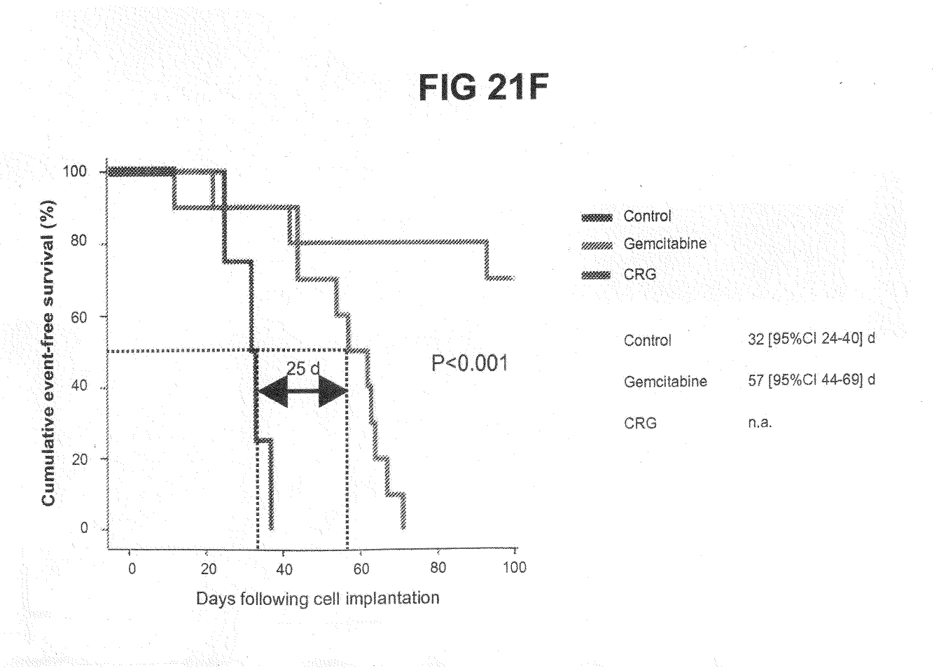

In order to test the identified combination therapy in a clinically most relevant setting, a model of established orthotopic pancreatic cancer was used in this experiment. One week after cell implantation, the tumour take rate was confirmed and tumour-bearing mice were randomized for treatment. The detailed experimental setup is depicted in FIG. 21A. Cyclopamine and Rapamycin were administered for only two weeks to minimize potential stem cell-associated side effects. Gemcitabine was given for a prolonged period of 11 weeks in analogy to clinical practice. Tumour volume was non-invasively measured on day 42 using a caliper (FIG. 21B), and was controlled via MR imaging on day 49 (FIG. 21C). Based on these positive results, the experiment was continued until day 100. White blood cell counts showed no evidence for undesired effects of the stem cell inhibitors on the hematopoietic system (FIG. 21D). Intriguingly, tumour take rate on day 100 was dramatically reduced in the CRG group as compared to Gemcitabine alone (FIG. 21E). All control animals bore large, life-limiting tumours and succumbed within one month after tumour implantation. Gemcitabine alone significantly prolonged survival of the animals due to inhibition of tumour growth. However, the animals' median survival was still severely limited with 57 days due to the 100% tumour take rate. Intriguingly, for the CRG group, long-term survival was significantly better compared to Gemcitabine alone (FIG. 21F). Only one CRG animal carried a detectable tumour and had to be prematurely sacrificed on day 44. One death on day 8 was related to the oral feeding procedure and therefore censored. The other two deaths in the CRG group were also unrelated to tumour growth rather than related to local infections (n=1) or remained obscure without apparent pathologies during necropsy (n=1). In summary, out of 11 animals, 63% of the CRG animals reached the 100-day follow-up time point whereas none of the animals in the Gemcitabine alone group survived beyond day 71.

### Example 3.6

### Discussion of the Results of Example 3

Considering the still devastating prognosis of patients with pancreatic cancer the development of novel therapeutic strategies is a prerequisite to eventually achieve a better outcome. Previous studies demonstrated that pancreatic cancers contain a rare population of undifferentiated cells that express CD133, are exclusively tumorigenic, and, most importantly, are highly resistant to chemotherapy. Treatment of pancreatic cancer with the standard chemotherapeutic agent Gemcitabine is not capable of eliminating CSC, but rather leads to a relative increase in their numbers indicating its preferential effect on more differentiated and rapidly proliferating tumour cells. Therefore, although more differentiated cells represent the bulk of the tumour, their elimination will not lead to the eradication of the tumorigenic potential of the tumour as that is limited to the cancer stem cell population. According to the present invention it was demonstrated for the first time that inhibition of either the SHH pathway or of mTOR signalling together with Gemcitabine leads to a significant reduction of pancreatic CSC and exclusive inhibition of the SHH pathway and mTOR signalling, i.e., the combined inhibition of both pathways, together with Gemcitabine is capable of significantly depleting the pancreatic CSC pool. Indeed, in vitro combination therapy resulted in total abrogation of the tumorigenic potential of the cells as evidenced by subsequent in vivo transplantation studies. Most importantly, however, in vivo treatment of established orthotopic pancreatic tumours using this triple therapy (and also of the double therapy) significantly enhanced long-term event free survival during long-term follow-up.

The surface antigen CD133 has been used for the identification of a subpopulation of pancreatic cancer cells that is highly enriched for tumour-promoting CSC both in primary pancreatic cancer cells as well as in the in vivo passaged pancreatic cancer cell line L3.6pl Importantly, various different CD133 antibodies are commercially available, which vary considerably with respect to targeted epitopes and binding characteristics. For the present studies, the present inventors used a Miltenyi monoclonal antibody recognizing a glycosylated extracellular epitope (AC133) while others have used different monoclonal and polyclonal antibodies against CD133, respectively, in pancreatic cancer. Apart from other methodological differences in sample acquisition and processing that may also have to be considered, the use of these different antibodies can translate into significantly different findings. Two studies using different CD133 antibodies for the histological assessment of pancreatic cancer have led to opposing results with respect to the prognostic information that can be derived from the expression pattern of CD133 (see Immervoll et al., (2008), BMC Cancer 8, 48; and Maeda et al., (2008), Br J Cancer 98, 1389-1397).

Using flow cytometry, the present inventors have thoroughly characterized the CD 133 expression patterns of primary pancreatic cancer tissues and in vivo passaged L3.6pl cells. Using this marker, the data obtained strongly support the cancer stem cell concept not only for fresh primary cancer cells but also for this specific pancreatic cancer cell line. Li and colleagues (2007) have used CD44 and CD24 to successfully enrich for tumour-promoting pancreatic CSC (see Li et al., (2007), Cancer Res 67, 1030-1037.) It is important to note that none of these markers should be universally applied to existing pancreatic cancer cell lines without in vivo validation. Indeed, the combination of CD44 and CD24 cannot be used to enrich for a CSC population in most pancreatic cancer cell lines as virtually all cells express both markers. By means of exception, ASPC cells demonstrate only a very small fraction of CD44⁺ CD24⁺ cells, which can also be successfully targeted by the presented CRG triple therapy. These data suggest that the combination of CD44 CD24 could also be useful for the identification of CSC in selected pancreatic cancer cell lines. Of course, experiments in cancer cell lines need to be studied due to the limited number and size of available tissue specimens from patients with pancreatic cancer but should be regarded rather as hypothesis generating than hypothesis proving. The ultimate test remains the analysis of fresh primary human samples. Intriguingly, CRG triple therapy significantly reduced both CD133⁺ cells as well as CD44⁺ CD24⁺ cells. Considerable overlap between the two populations defined by different marker sets has been observed.

This heterogeneity for pancreatic cell lines is actually also reflected by a heterogeneity of fresh patient derived samples with respect to the expression of markers that have been used for the enrichment of CSC. Indeed, the present inventors have observed that a small subset of patients is either negative for CD44, CD133, or both. This heterogeneity may be related to insufficient expression of these markers, modulation of the expression pattern during the necessary digesting process following harvesting of the primary tissue, or may actually indicate that the CSC hypothesis is not a universal model that can be applied to all individual patients' samples. Indeed, whether an individual tumour follows the CSC model or not may depend on whether the initializing mutation occurred in the stem cell compartment or in more differentiated progenitor cells.

Stimulated by the possible crucial role for activated SHH signalling in pancreatic cancer (stem) cells the present inventors focused the initial investigations on this pathway. SHH mediates its biological effects via inhibition of the transmembrane receptor Patched. While Patched exerts inhibitory effects on Smoothened in the absence of SHH, binding of SHH to Patched results in activation of Smoothened with subsequent transcription of the SHH target genes among the Gli protein family. Inhibition of hedgehog signalling by Cyclopamine has been shown to attenuate pancreatic cancer growth in vitro and in vivo (Thayer et al., 2003, Nature 425, 851-856.) Unfortunately, although these data suggest that this pathway plays a critical role in the promotion of pancreatic cancer, SHH inhibition alone did not result in a complete abrogation of the tumorigenic activity. Indeed, previous studies already suggested that SHH inhibition has a more preferential effect on cells responsible for invasion and metastatic seeding (Feldmann et al., 2007, Cancer Res 67, 2187-2196). The present inventors observed a significant reduction in the migratory activity of the cells following in vitro treatment with Cyclopamine alone. However, presumably because the CSC population was never completely eliminated, the metastatic activity in vivo was only partially reduced by single-agent therapy with Cyclopamine While a stronger effect was reported (Feldmann et al., 2007, supra) it is important to note that these investigators used a model of orthotopic pancreatic cancer while the present inventors have used a systemic infusion model to primarily focus on the seeding capabilities of the cells following exposure to Cyclopamine. In the present experiments, only the combined pretreatment using Cyclopamine and Gemcitabine resulted in a significant reduction of the metastatic activity. Consistently, this combined treatment also resulted in the complete elimination of the CD133+CXCR4+ CSC, which the present inventors have defined as the metastasis-driving CSC population and, therefore, have also been termed migrating CSC.9 The above data suggest that Cyclopamine, unlike conventional chemotherapeutic agents, preferentially targets the migrating CSC subpopulation, which has been shown to be responsible for the metastatic spread of pancreatic cancer (Hermann et al., 2007, supra).

During extensive screening studies using a large variety of inhibitors targeting stem cell-relevant pathways, the present inventors also surprisingly identified the mTOR pathway as another promising candidate. As discussed above, mTOR, the target molecule of a complex signal transduction pathway, is a serine/threonine-kinase belonging to the PI(3)Kinase superfamily. The PI(3)K pathway is highly branched, but activates mTOR among other downstream effectors. (Inoki et al., 2005, Nat Genet 37, 19-24.) It was only recently shown that deletion of the signalling molecule Pten, which is localized upstream of mTOR, results in depletion of normal hematopoietic stem cells while promoting expansion of leukemia-initiating cells (Yilmaz et al., 2006, Nature 441, 475-482) Intriguingly, these effects were mostly mediated through mTOR as Rapamycin, the naturally occurring inhibitor of mTOR, not only depleted leukemia-initiating cells, but also restored normal hematopoietic stem cell function. Notably, mTOR signalling was also just recently confirmed to be critical for breast cancer stem cell survival and proliferation. (Zhou et al., 2007, Proc Natl Acad Sci USA 104, 16158-16163) Together these data support the notion that it may become possible to distinguish between the mechanisms regulating the maintenance of normal compared to cancer stem cells.

In line with these findings, Rapamycin alone already results in a significant decrease of CD133+ pancreatic CSC Immunohistochemistry for phospho-s6-ribosomal protein, which is located downstream of mTOR, confirmed that the mTOR pathway is only active in a small subset of cells including the CSC population and can be profoundly inhibited by Rapamycin. These data also provide a rationale for the rather modest anti-proliferative effect reported in previous studies, which have not been focusing on the CSC subpopulation. (Guba et al., 2002, Nat Med 8, 128-135) Despite a reduction in the CSC content to an even greater extent as compared to SHH inhibition, mTOR inhibition alone also did not translate into a reduced in vivo tumorigenicity underlining the importance of functional assays including in vivo tumorigenicity for this kind of investigation. Depending on the true CSC content of the CD133+ cell population, one would expect that only a very dramatic reduction of the CSC content would be capable of translating into a clinical benefit for the patient.

Since none of inhibitors tested for the purposes of the present invention achieved complete elimination of CD133⁺ cells on its own, successful targeted CSC elimination may indeed require the inhibition of multiple stemness pathways as a consequence of their redundancy and/or non-exclusiveness. Interestingly, a treatment regimen combining the inhibition of the SHH pathway and the mTOR pathway only modestly, but non-significantly reduced tumour take-rate. Only the addition of Gemcitabine, which on its own did not generate any effect with respect to reduction in tumorigenicity, surprisingly led to a complete elimination of the cells' tumorigenic capacity. Therefore, it is intriguing to speculate that the primary mechanism of action for this combination therapy may be loss of stemness.

Of course, there are some limitations inherent to the design of the present study. Xenograft models are not ideal to study tumour biology in vivo but are the only option for studying human pancreatic CSC. Since the utilized inhibitors for stem cell-related pathways are not specific for CSC, side effects on the normal stem cell compartment may occur. For Rapamycin, lymphatic complications resulting from a disruption of VEGF-mediated lymphangiogenesis and lymphatic recovery have also been described (Huber et al., 2007, supra) For SHH inhibitors, clinical phase I and II trials are still on-going but preliminary analyses suggest a reasonable safety profile. In order to minimize possible side effects, the present inventors administered the inhibitors for only two weeks. In their experiments, CRG treated mice showed no evidence for leucopenia as the most likely side effect via depression of the hematopoietic system. Some animals were lost due to repetitive infections. Thus, while two weeks of therapy were apparently sufficient to affect CSC in vivo, the present inventors observed only modest toxicity for the combination therapy at the given doses.

In conclusion, the above data are well in line with the CSC hypothesis as discussed initially implicating that tumours are generated and promoted by a small subset of undifferentiated cells with the ability to self-renew and differentiate into the bulk tumour cell population. Following standard chemotherapy, CSC still remain viable and maintain their tumorigenic capacity. Theoretically, even a single CSC should be able to reproduce an entire tumour (Zucchi et al., 2007, Proc Natl Acad Sci USA 104,10476-10481) emphasizing the definitive need for a therapy that truly eliminates all CSC as the suspected root of the disease. Here we now demonstrate that treatment with a combination of Cyclopamine, Rapamycin and Gemcitabine is indeed able to virtually eliminate pancreatic CSC both in vitro and in vivo. As Rapamycin has already been clinically approved for patients with kidney transplants and SHH inhibitors are presently under clinical investigation in other tumour entities, further preclinical investigation of this novel treatment modality for the treatment of pancreatic cancer is feasible and warranted.

### Example 3.7

### Experimental Procedures for Example 3 Above

Human pancreatic cancer cell line. The highly metastatic human pancreatic cell line L3.6pl was maintained in DMEM medium (Invitrogen, Karlsruhe, Germany) with 12% Fetal Calf Serum (Biochrom, Berlin, Germany), Glutamax (Invitrogen), non-essential amino acids, and vitamins (all from PAN, Aidenbach, Germany) (Hermann et al., 2007, supra). Cultures were kept no longer than 4 weeks after recovery from frozen stocks. CSC spheres were cultured in DMEM-F12 supplemented with B27 (Gibco, Karlsruhe, Germany) and FGF-2 (PeproTech EC, London, United Kingdom) (Hermann et al., 2007, supra).

Primary human pancreatic cancer cells. Human pancreatic cancers were obtained with written informed consent from all patients. Tissue fragments were minced with scissors into small (1-2 mm³) fragments. Enzymatic digestion was performed using a mixture of DMEM medium and collagenase (Stem Cell Technologies, Vancouver, Canada) for 90 min at 37° C. (Hermann et al., 2007, supra).

Cytometry. Pancreatic CSC were identified by CD133/1-APC or CD133-PE (Miltenyi, Bergisch-Gladbach, Germany) (Hermann et al., 2007, supra). Side population experiments were performed as described by Goodell et al. (1996) (see Goodell et al., 1996, J Exp Med 183, 1797-1806) and cells were identified by exclusion of the vital dye Hoechst 33342. To detect activation of the PI3K/Akt/mTOR pathway by means of the phosphorylation status of the s6 ribosomal protein (s6rp), cells cultivated with standard medium were stained with a rabbit anti-phospho-S6 ribosomal protein antibody (Cell Signalling Technology, Danvers, Mass.).

rtPCR. RNA was primed with oligo(dT) and reverse transcribed using the following primers: FSequence: CTTTCATCAACTCGCGATGC, RSequence: GCTCATGGTGCCAATGGAG (Operon Biotechnologies, Cologne, Germany). Semi-quantitative PCR for human Gli1 (40 cycles) and GAPDH (25 cycles) as housekeeping gene was done using a light cycler PCR system (Roche).

Transmigration assay. A total of 5×105 isolated tumour cells were resuspended in 250 μl DMEM containing 5% FCS and placed in the upper chamber of a modified Boyden chamber filled with Matrigel™ (BioCoat™, BD Biosciences, Heidelberg, Germany). The lower chamber contained the same medium supplemented with 100 ng/mL SDF-1 (R&D Systems, Minneapolis, Minn.). Transmigrated cells were counted after 24 hours (Hermann et al., 2007, supra).

Animals and Orthotopic Implantation of Tumour Cells. Single-cell suspensions were orthotopically implanted into the pancreas of female NMRI nu/nu mice (Janvier, Le Genest-Saint-Isle, France) (Hermann et al., 2007, supra). Size and weight of the pancreatic tumours were monitored.

In vitro treatment of pancreatic cancer cells. Pancreatic cancer cells were treated for up to 96 h with the following substances (single treatment or in combination): Gemcitabine (24 h) 100 ng/mL (Lilly, Muenster, Germany), Cyclopamine 10 μM (Biomol, Plymouth Meeting, Pa.), or Rapamycin 100 ng/mL (Wyeth, N.Y.). For the evaluation of the in vivo tumorigenicity, 106 L3.6pl cells were pretreated with the respective regimen and the remaining viable cells were orthotopically implanted into the pancreas of nude mice and evaluated on day 35. For the systemic infusion assay, cells were labelled with the Qtracker 800 labelling kit (Invitrogen) according to the manufacturer's instructions, and 5×10⁵ cells were injected intravenously into mice. After 4 weeks, explanted lungs were scanned for metastases with a near-infrared imaging platform (Odyssey, Li-cor, Lincoln, Nebr.). Positive signals were verified by histology (H&E staining).

In vivo treatment of established pancreatic cancers. Seven days after orthotopic implantation of L3.6pl cells, mice were randomized to the respective treatment groups. Gemcitabine was administered biweekly (125 mg/kg i.p.). Cyclopamine was used as previously described at 25 mg/kg by oral gavages twice daily (see Feldmann et al., 2007, Cancer Res 67, 2187-2196). Rapamycin (5 mg/kg/day; Wyeth, Madison, N.J.) was orally administered via the drinking water as reported previously (Huber et al., 2007, supra).

Positron Emission Tomography (PET). Twenty MBq of 2-Deoxy-2-[18F]-fluoro-d-glucose (Technical University, Munich, Germany) were systemically injected in a volume of 0.2 mL. After an uptake time of 60 minutes, images were acquired for a total acquisition time of 20 minutes using a Siemens Inveon P 120 scanner (Siemens, Erlangen, Germany).

Magnetic Resonance Tomography (MRT). Mice were analyzed with a 3-Tesla MRI system (Magnetom Tim Trio, Siemens, Erlangen, Germany) using a dedicated small animal coil and T2-weighted scanning.

Statistical analysis. Treatment groups were compared by independent samples t test. In case of non-normal distribution, the Mann-Whitney U test was used. Pair-wise multiple comparisons were performed with the one-way ANOVA (two-sided) with Bonferroni adjustment. All analyses were performed with SPSS 16.0 (SPSS Inc., Chicago, Ill.).

### Example 4

### Inhibition of the Nodal/Activin Signalling Pathway Using SB431542

### Example 4.1

### Expression of Activin and Nodal in Pancreatic Neoplasms

In order to investigate whether Activin A, Nodal and its co-receptor Cripto are expressed in pancreatic neoplasms, pancreatic cell lines L3.6 pl, MiaPaCa 2, BxPC3, CFPAC and Hs766T, as well as primary patient samples of healthy and malignant tissues were screened for their expression using rt-PCR. A strong expression of Nodal and marked expression of Activin A and Cripto was found in cell lines and tumors compared to healthy tissues.

To further assess the localization of the proteins, tissue sections of primary patient samples were investigated using immunofluorescence. It was found that Activin, Nodal, and Cripto are expressed in most malignant sections, while only modest or no expression was observed in healthy tissues.

### Example 4.2

### Inhibition of Activin/Nodal Signaling Targets the Tumorigenic Population in Pancreatic Cancer

Using SB431542, a specific inhibitor of the receptors for Nodal and Activin, ALK-4 and -7 respectively, it was investigated whether the content of the CD133(+) population is affected by inhibition of Activin/Nodal signaling. As mutations affecting the TGF-superfamily pathway are very common in pancreatic neoplasms, pancreatic cell lines harboring different mutations in this pathway were screened (Table 1).

TABLE 1
Mutations in components of the TGF-B superfamily
pathways in investigated cell lines

| Cell Line | Mutation in TGF-B pathway | Comment |
|---|---|---|
| L3.6pl | homozygous deletion of SMAD4 | cell line contains ALK-4/-7 |
| BxPC3 | homozygous deletion of SMAD4 | |
| AsPc1 | missense mutation | cell line contains ALK-4/-7 |
| MiaPaCa2 | deletion of TGF-B type II | cell line contains ALK-4/-7, |
| | receptor | cell line contains SMAD4 |

While L3.6pl and MiaPaca cells responded to treatment with a decrease in the CD133(+) sub-population as shown by flow cytometry, BxPC3 cells showed no response in terms of CD133 content. Addition of Gemcitabine to anti-Nodal/Activin treatment resulted in a nearly complete depletion of the tumorigenic CD133 positive cells in those cell lines that initially responded to treatment with SB431542, while no effect was observed in BxPC3 cells. These data suggest that the status of SMAD4 mutation is not functionally relevant for the treatment effect of SB431542, whereas the deletion of TGF-B type II receptor is apparently highly relevant. Without being bound thereto, this indicates a Nodal/Acticin mediated pathway being independent from SMAD4 as an underlying mechanism.

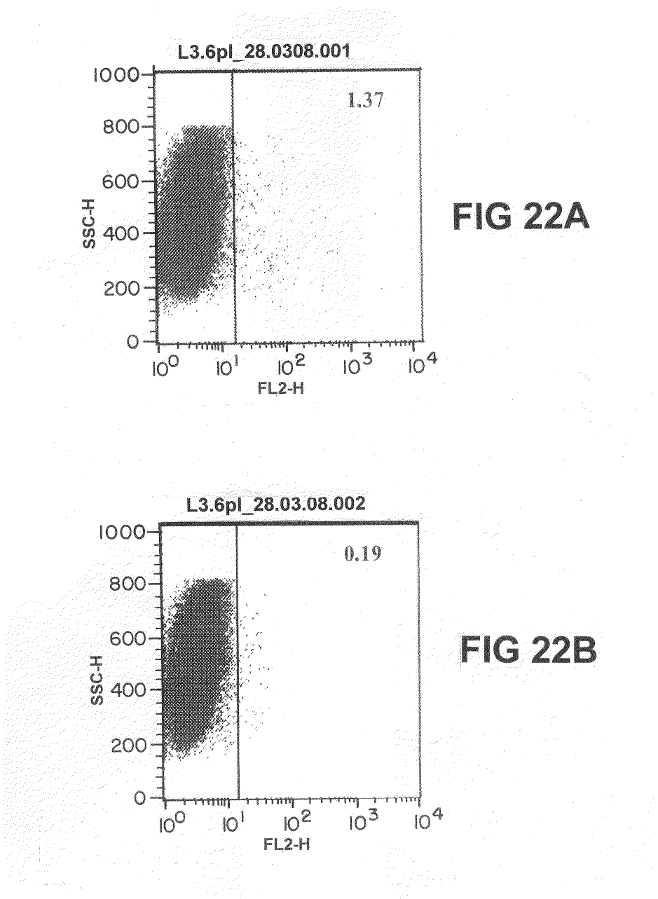

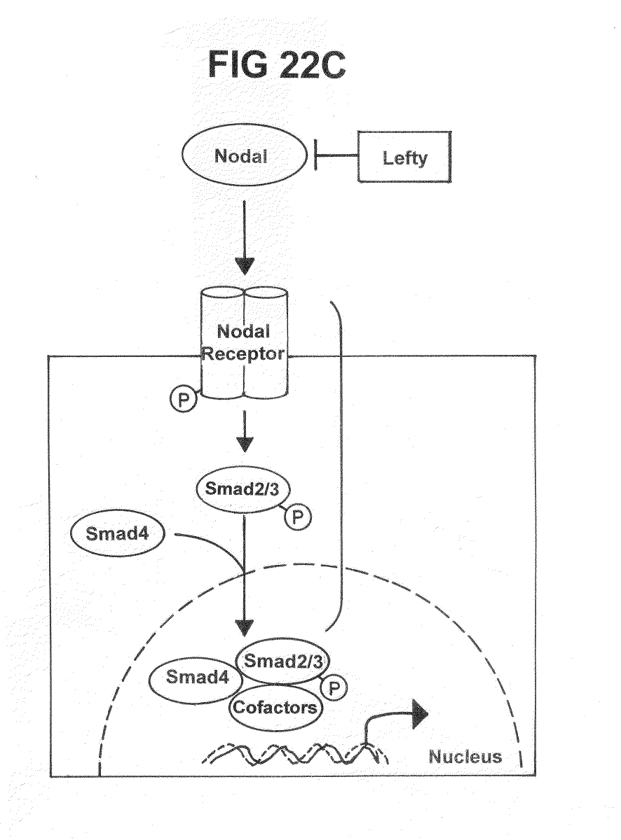

Furthermore, cells in culture were subjected to extracellular antagonists of Nodal (Lefty) and Activin A (Follistatin) to see whether both components are functionally relevant for the maintenance of the CD133 positive CSC compartment. Lefty decreased the CD133 positive population to a significant extent (see FIGS. 22A-22B).

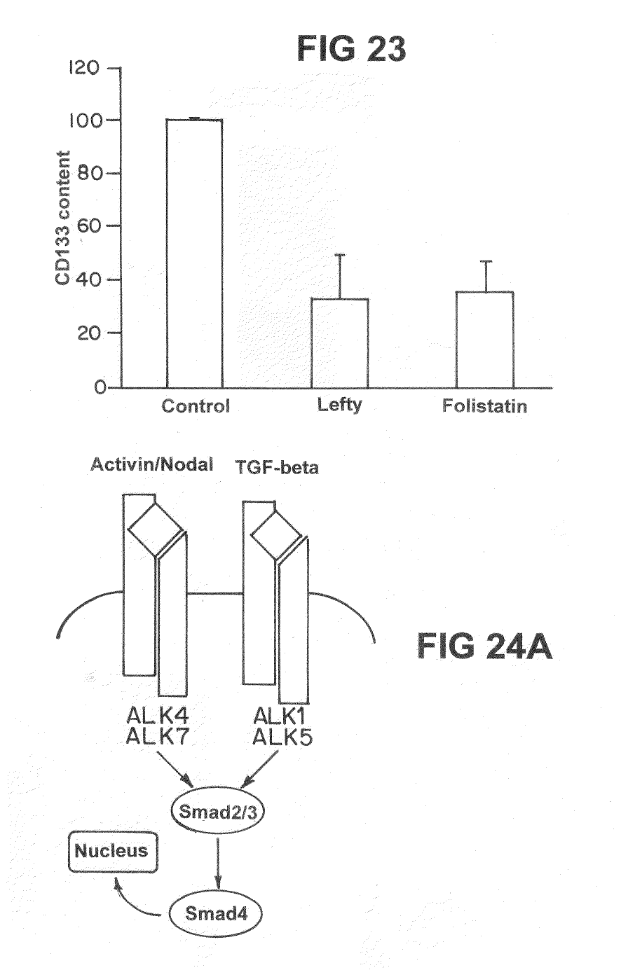

Intriguingly, it was found, that found that Folistatin as an inhibitor of Activin signaling resulted in a reduction of CD133 positive CSC that was similar to the reduction seen for Lefty (FIG. 23). Even more importantly, like treatment with SB431542, the treatment effect was by far more effective in depleting CSCs when cytostatic therapy was added.

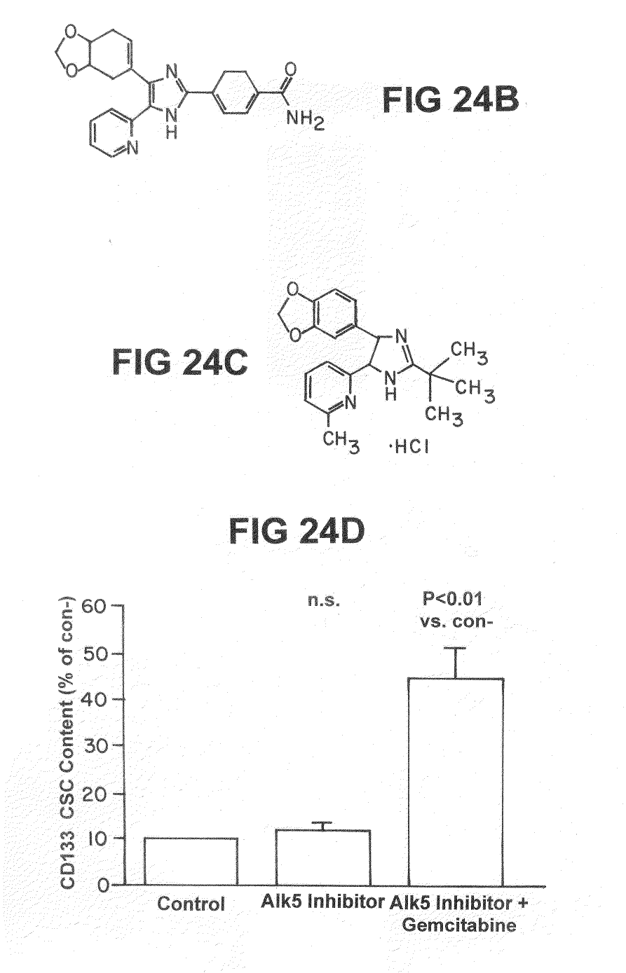

Additionally cells were treated with SB505124, a preferential inhibitor of ALK-5 with lower affinity to ALK-4 and ALK-7, the receptor for TGF-β to clarify whether there is a significant contribution of TGF-β to these effects. However, neither alone nor in combination with Gemcitabine an effect of SB505124 on the CD133 content of L3.6pl cells was observed (see FIG. 24D).

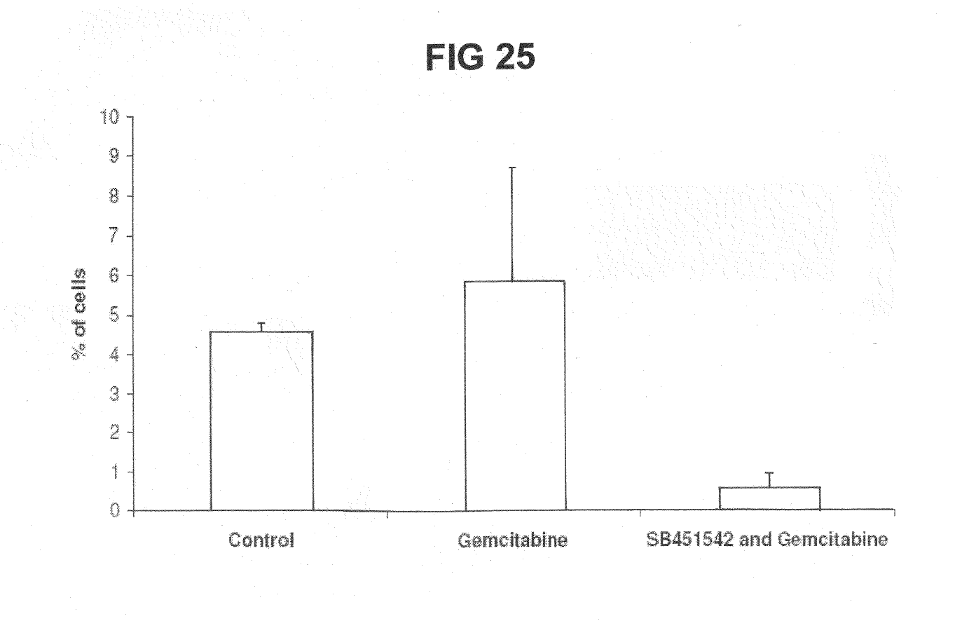

As Cancer Stem Cells (CSC) in Pancreatic Cancer have also been identified by combined expression of CD44 and CD24, the effect of treatment on this population using the AsPC 1 cell line was assessed (see FIG. 25) While this set of cells is also resistant towards Gemcitabine as witnessed by their relative increase in the overall population, addition of SB431542 to Gemcitabine also made these cells susceptible to treatment as shown by a drastic decrease of their relative number.

### Example 4.3

### Combined Therapy with SB431542 and Gemcitabine Permanently Affects Cancer Stem Cells

Since the use of SB431542 alone drastically reduced the CD133 content but did not affect the tumorigenicity after orthotopic implantation, the fate of cells after withdrawal of therapy was investigated. At 24 hours, the CD133 content of cells treated with the small molecule inhibitor alone already started to approach control level while it was not altered as compared to the base level in cells treated with Gemcitabine and SB431542. Even after 48 h no rebound in the CD133 content was observed after combined treatment, as opposed to SMI-treatment only which even resulted in an enrichment for CD133 compared to base level.

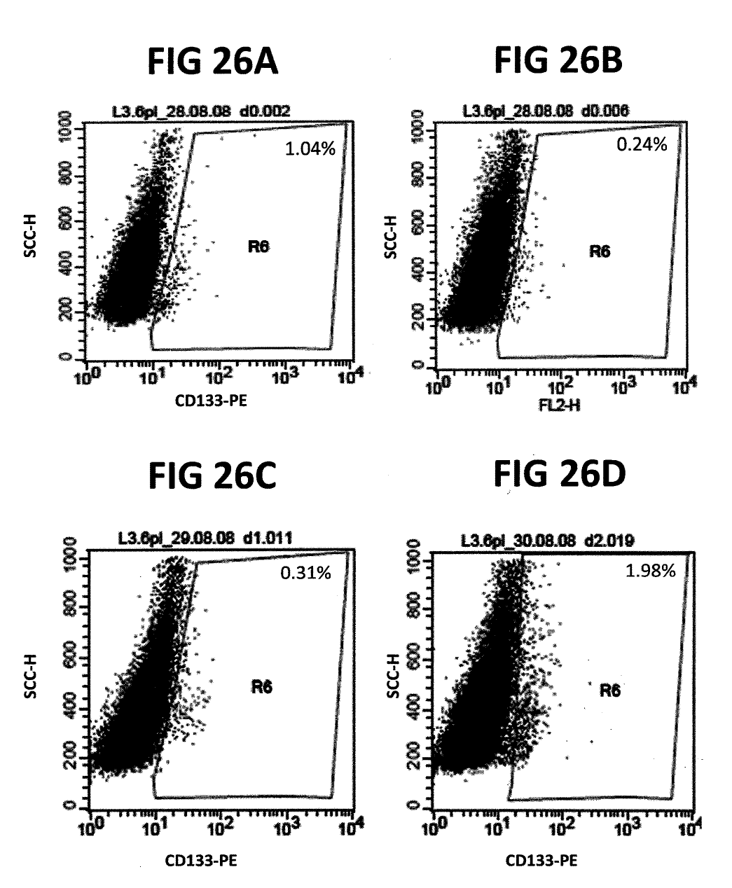

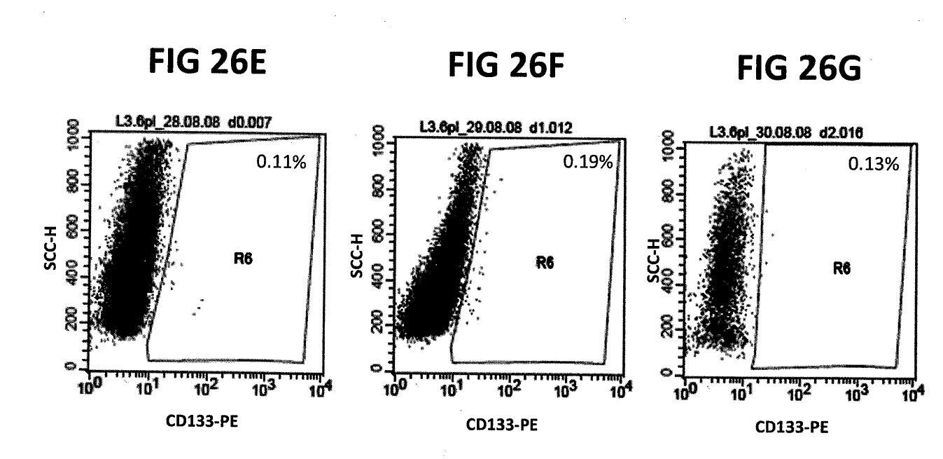

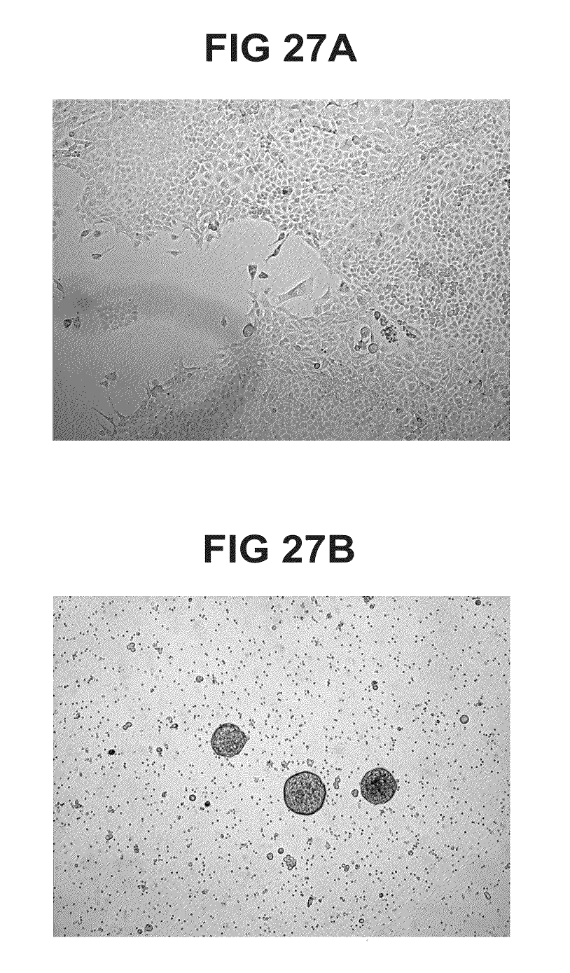

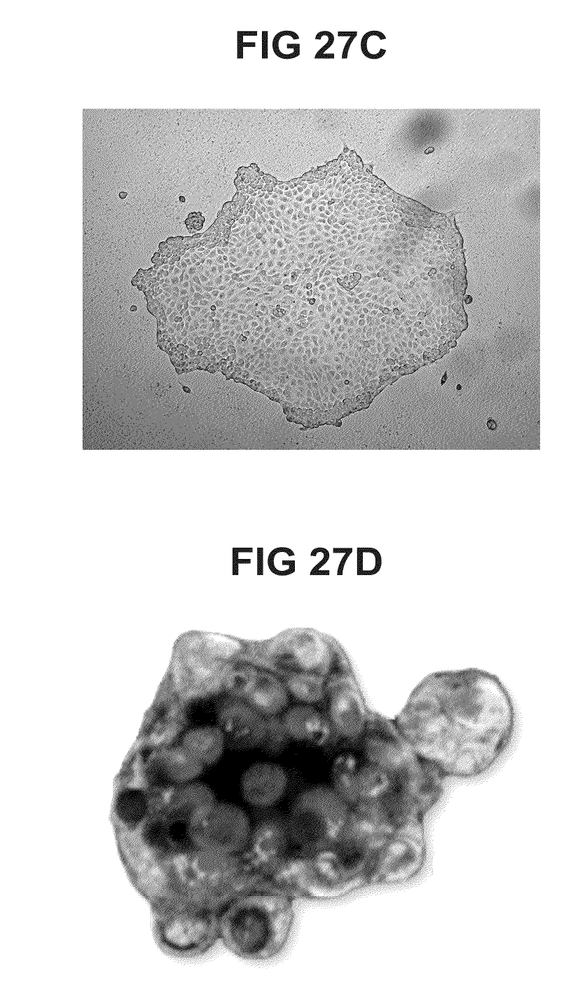

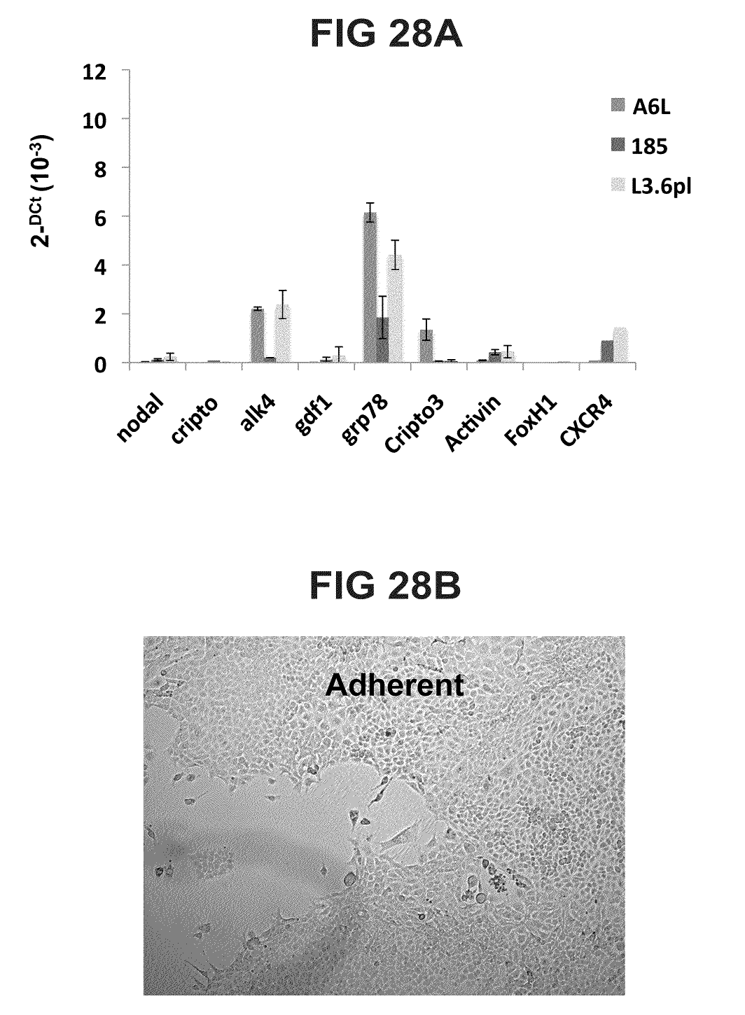

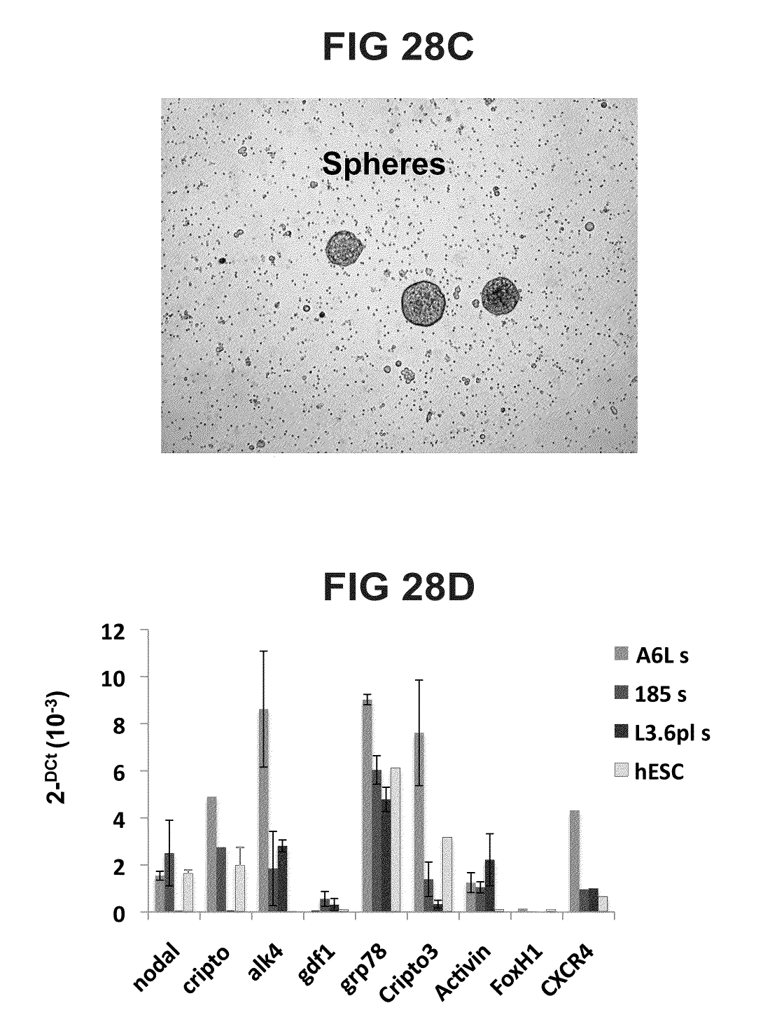

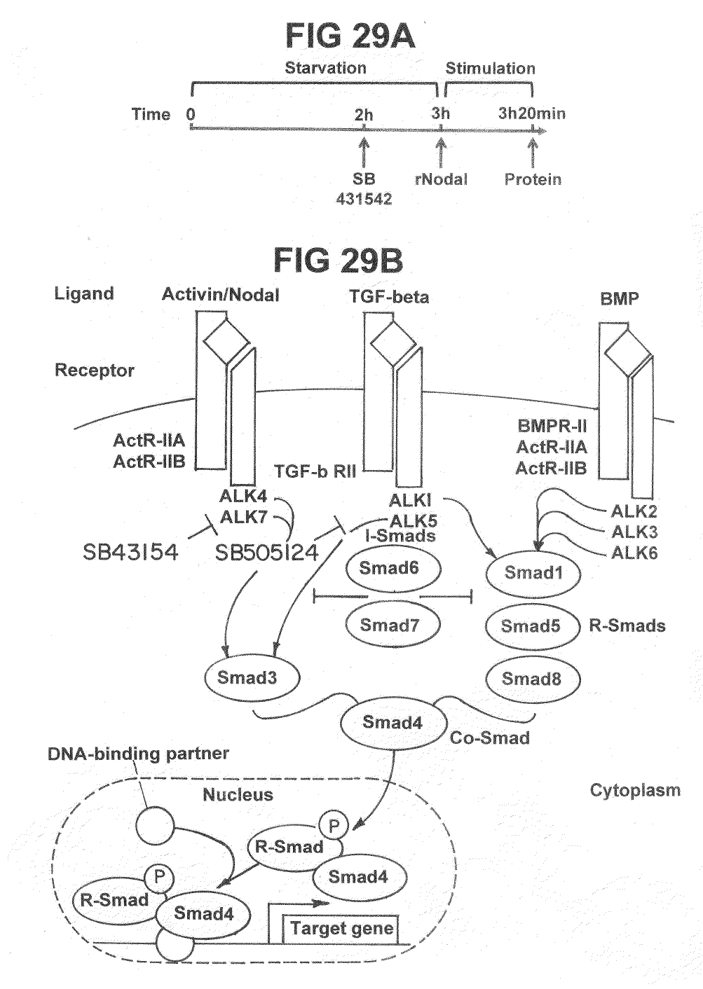

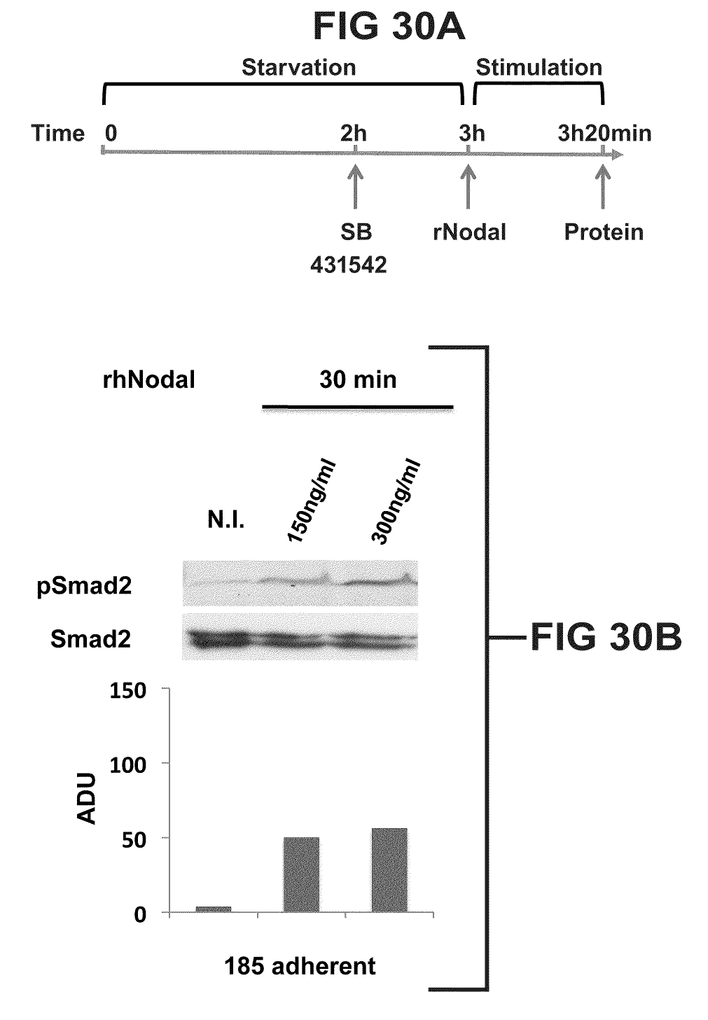

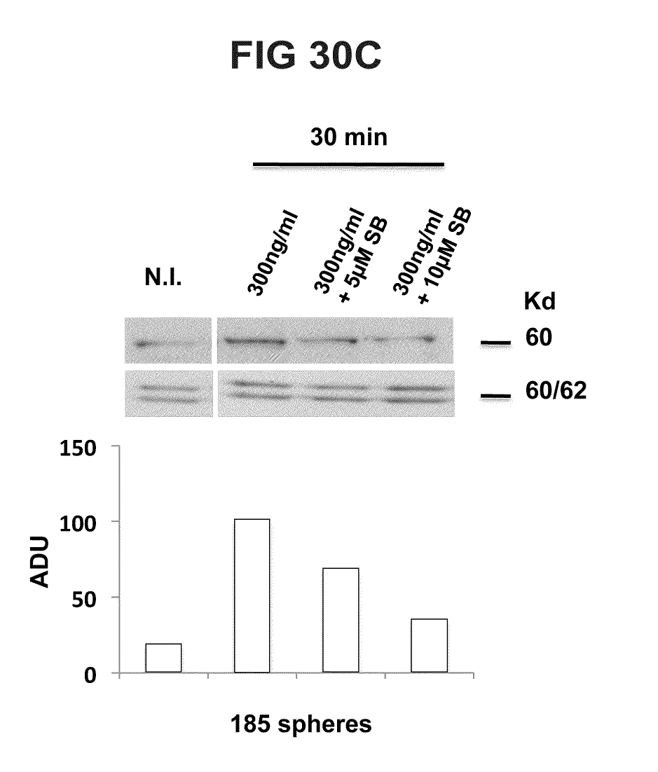

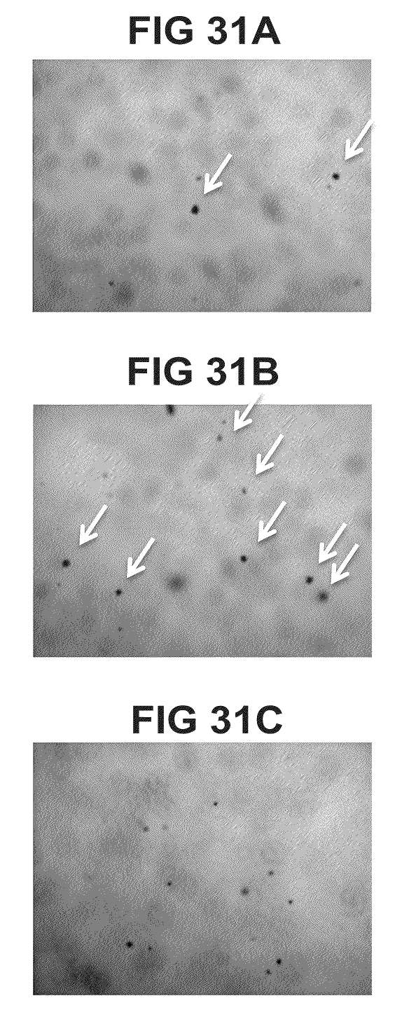

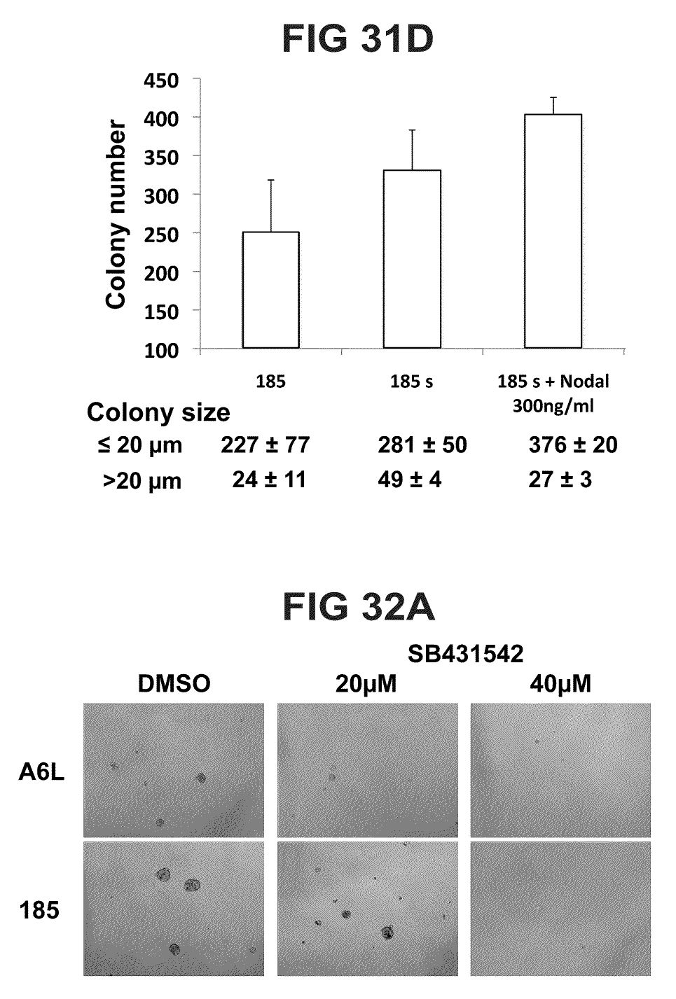

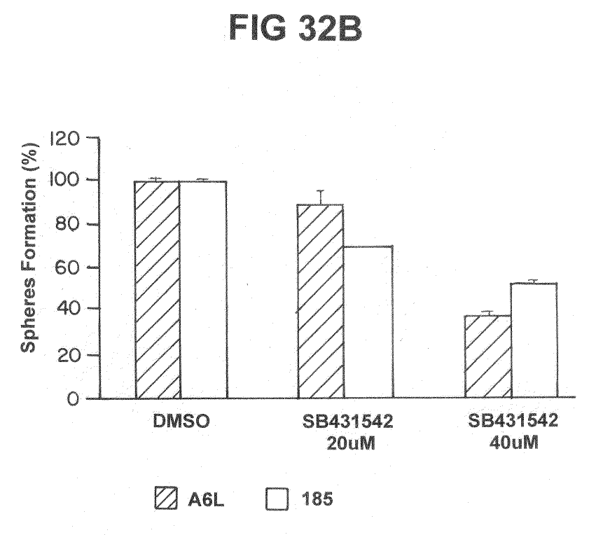

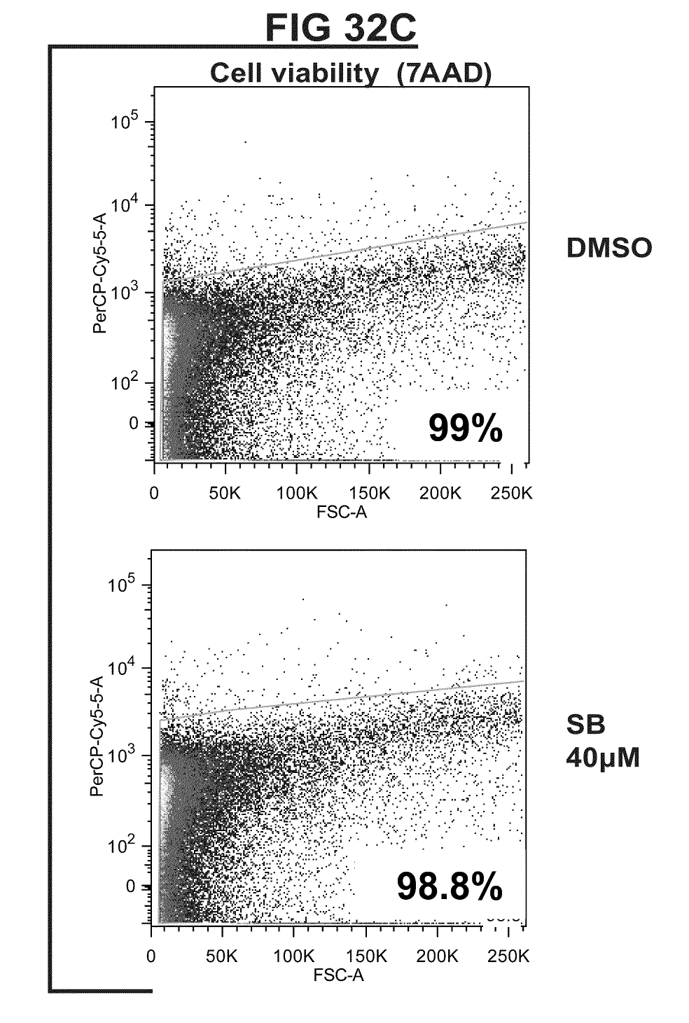

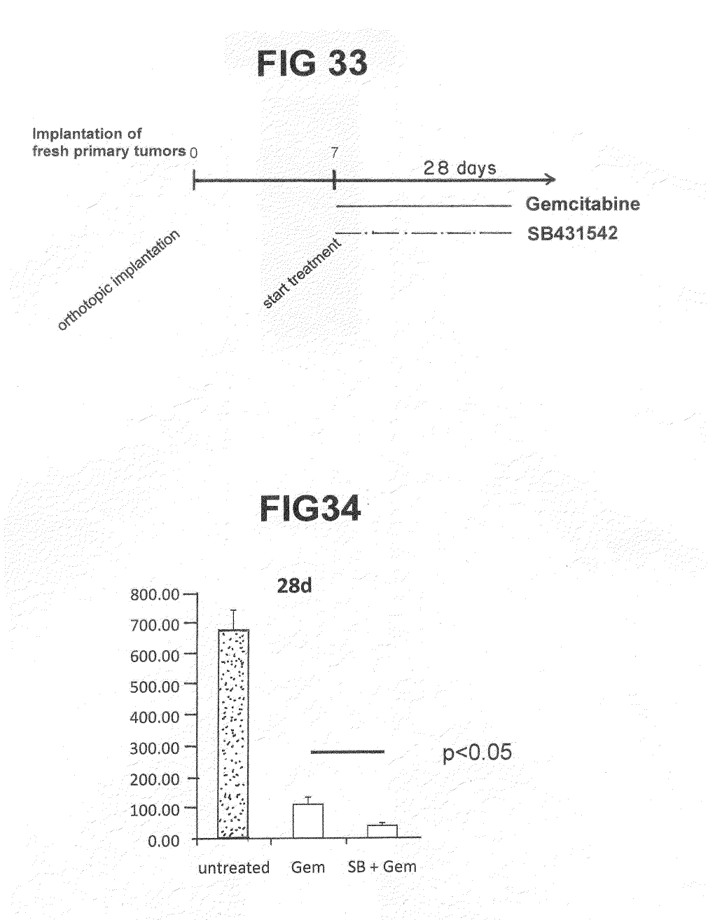

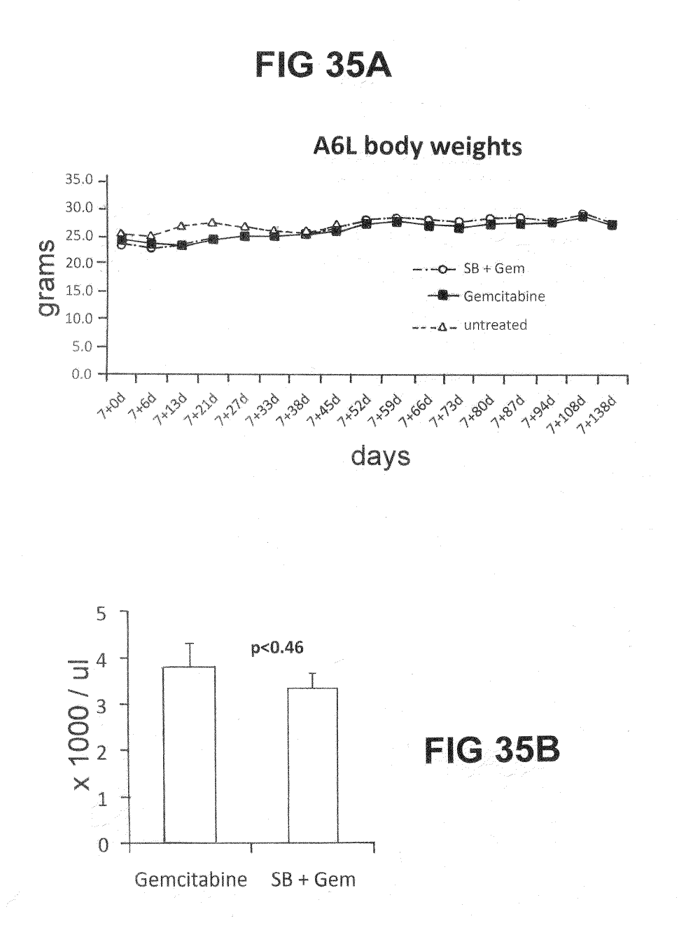

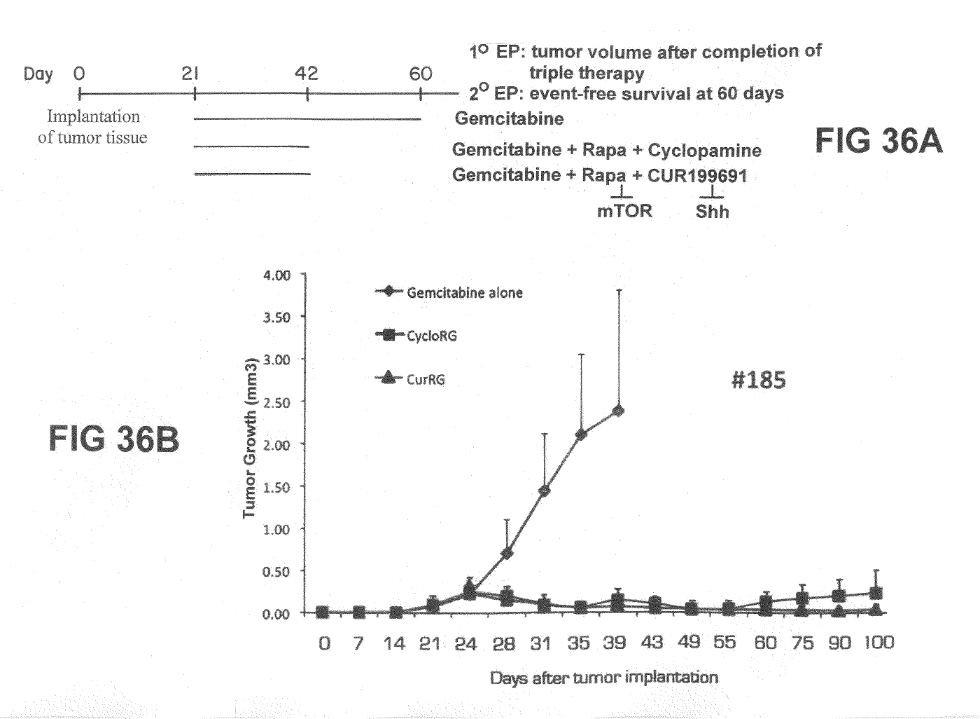

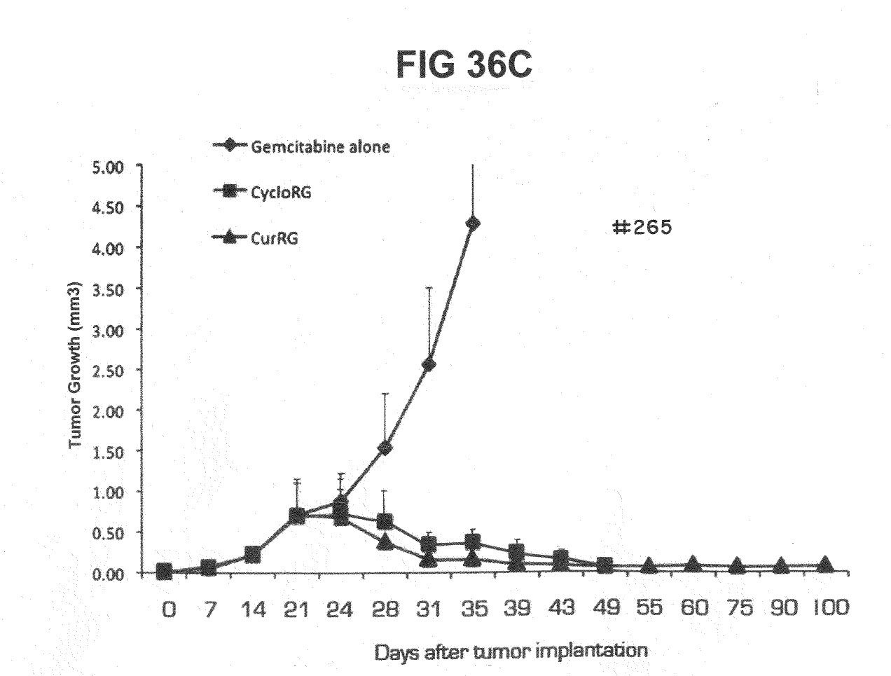

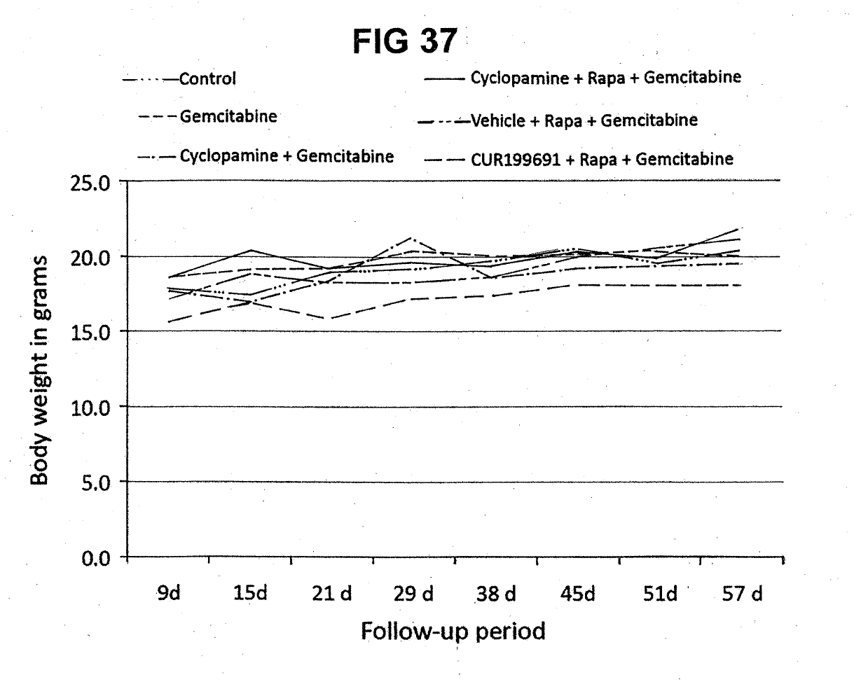

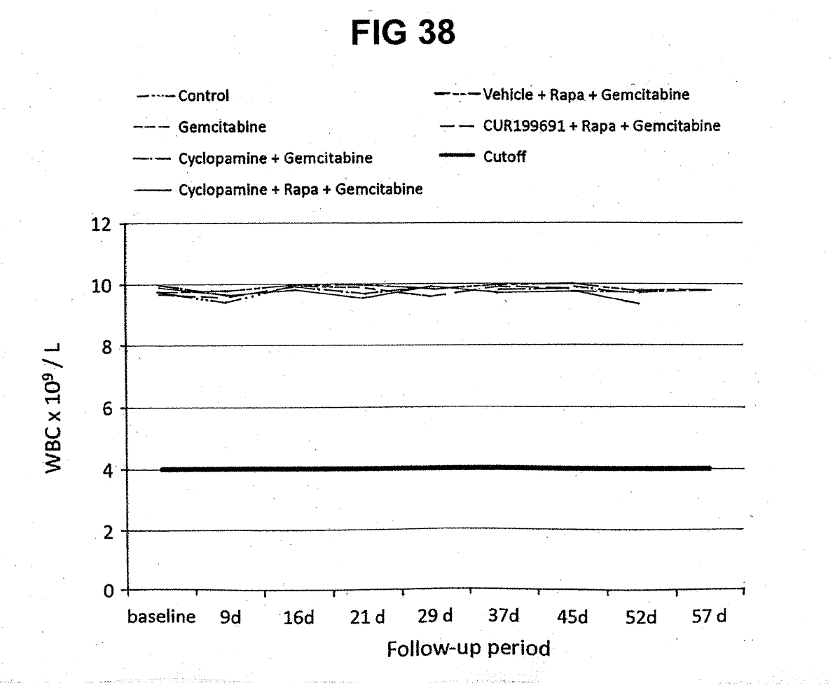

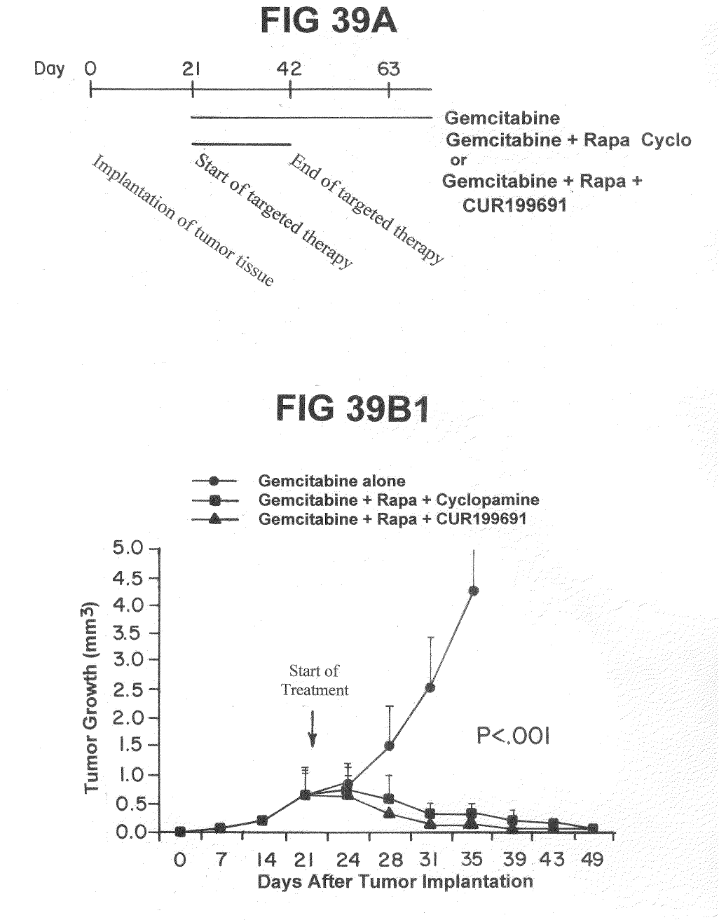

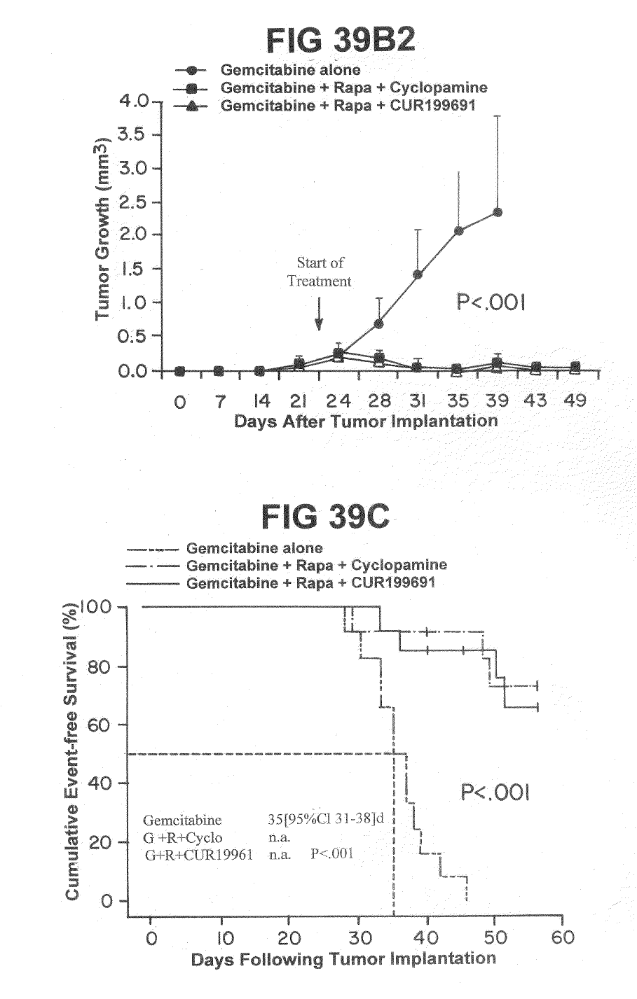

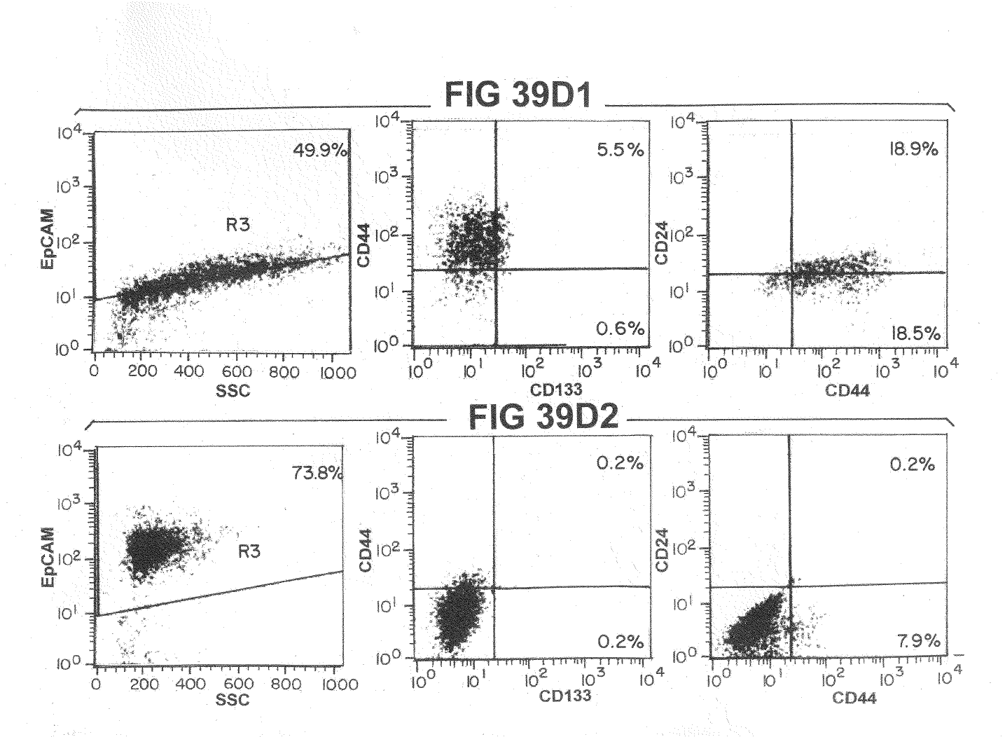

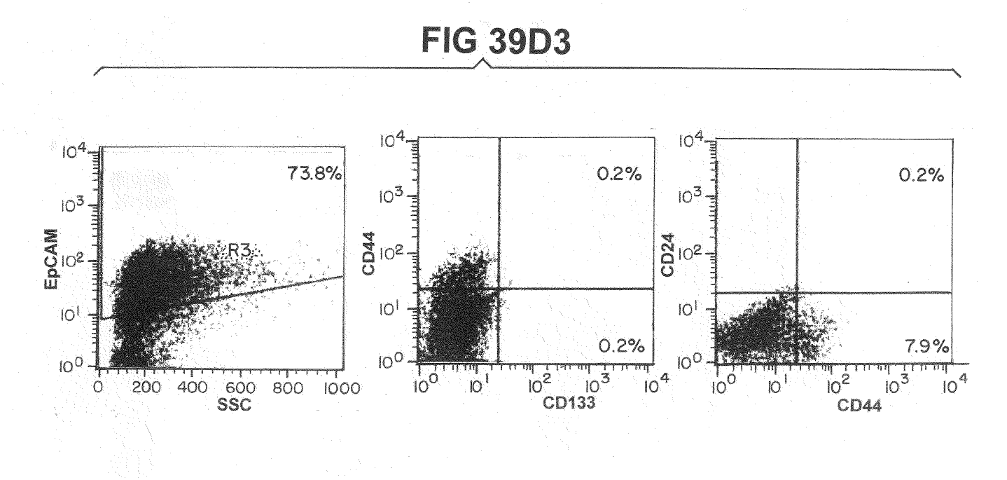

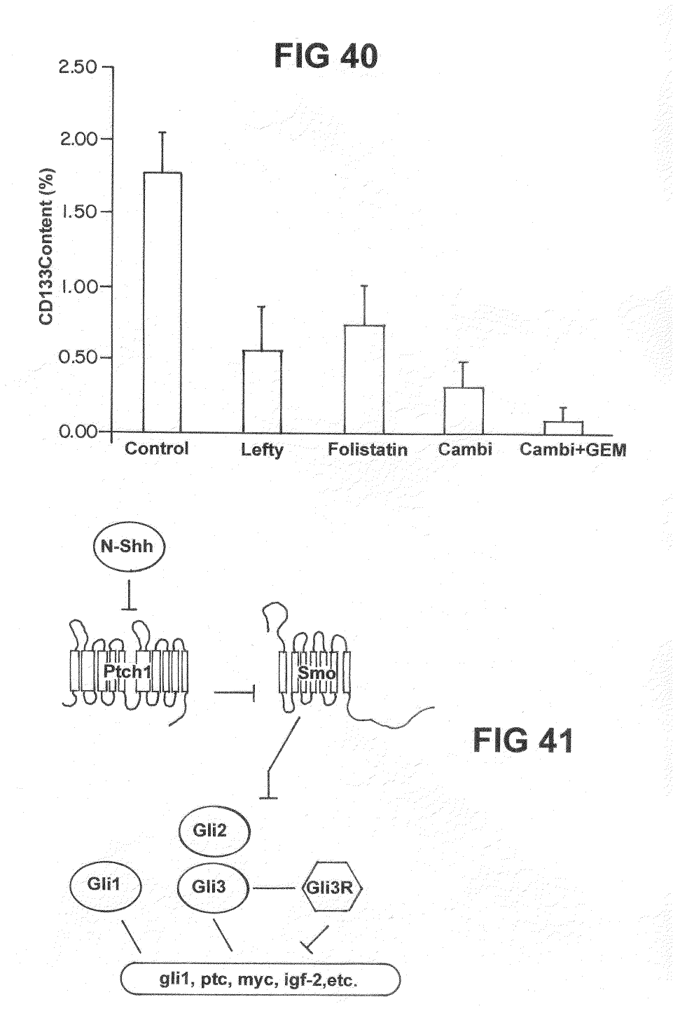

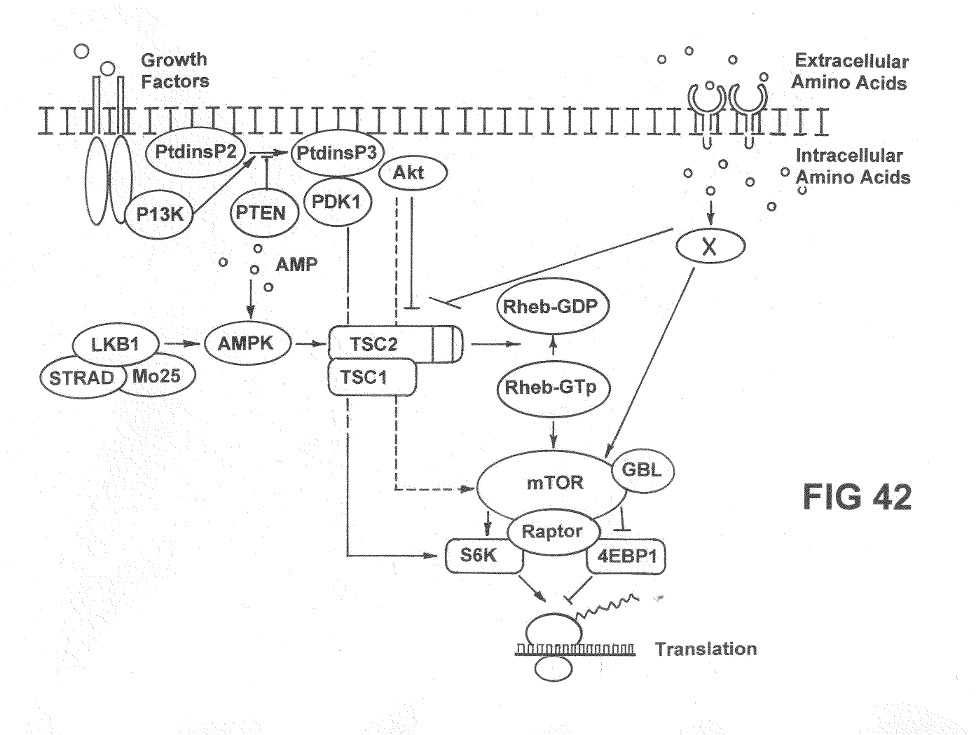

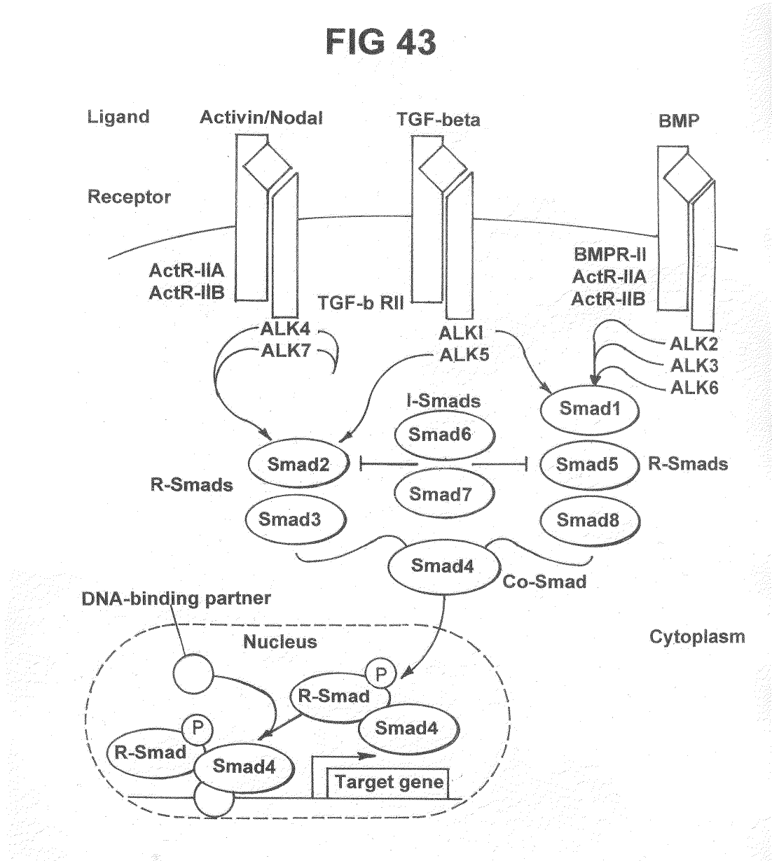

As the precise fate of CD133-positive cells during treatment with antagonists of Activin or Nodal was to be assessed, the cell cycle of these cells was investigated using a BrDU flow kit (see FIGS. 26A-26G). While treatment with SB431542 resulted only in a modest induction of apoptosis in this subset of cells, the combined treatment with Gemcitabine accounted for massive apoptosis. These findings strongly suggest that the effect on the tumorigenic population is largely mediated by induction of apoptosis in this subset of cells.

### Example 5

### In Vivo Treatment of Human Primary Pancreatic Tumor

The efficiency of double and triple active combinations according to the present invention on primary pancreatic tumors is further investigated. For this purpose, three groups of tumour-bearing mice (responders, intermediate and non-responders) obtainable by implanting tumors into the pancreas of these mice at day 0, receive a treatment using Gemcitabine (125 mg/kg biweekly for 6 weeks), Cyclopamine (2×25 mg/kg/day for 2 weeks), Rapamycin (5 mg/kg/day for 2 weeks) and the hedgehog antagonist CUR-0199691 (10 μM for 2 weeks) either with Gemcitabine alone, a combination of Gemcitabine, Rapamycin and Cyclopamine or a combination of Gemcitabine, Rapamycin and Cyclopamine and CUR-0199691. The Randomization and the start of the targeted therapy occur at day 21, the end of the targeted therapy is at day 35. The evaluation of the tumor volume occurs at day 63 and day 100, wherein during the 100 day follow-up a primary endpoint is set by an event-free survival of the mice, i.e., without death, aathia, cachexia, tumors >2 cm³ or infections), or a secondary endpoint set by tumor size or metastasis.

All publications, patents, and patent documents cited in the specification are incorporated by reference herein, as though individually incorporated by reference. In the case of any inconsistencies, the present disclosure, including any definitions therein will prevail.

1. An active substance combination comprising
(A) at least one nucleoside analog and/or a further anti-metabolitic agent capable to interrupt or interfere with DNA replication or synthesis
and
(B) either
(B1) at least one Nodal/Activin-Inhibitor
or
(B2) an active substance combination of
(B2a) at least one SHH inhibitor, and
(B2b) at least one mTOR inhibitor.

2. The active substance combination according to claim 1, wherein the nucleoside analog or the further anti-metabolitic agent is a
pyrimidine analogs;
purine analogs;
Purine antimetabolites;
Anthracyclines;
Folate analogs; or
Ribonucleotide reductase inhibitors.

3. The active substance combination according to claim 17, wherein the nucleoside analog is gemcitabine.

4. The active substance combination according to claim 1, wherein the Nodal/Activin-Inhibitor is SB431542, Coco-Protein, Nicalin-Protein, Nomo-Protein, Folistatin or Lefty.

5. The active substance combination according to claim 1, wherein the SHH-Inhibitor is Cyclopamine, Cyclopamine-KAAD, Jervine, CUR 61414, Forskolin, SANT-1, Arsenic Trioxide or CUR-0199691.

6. The active substance combination according to claim 1, wherein the mTOR-Inhibitor is selected from Rapamycin, Temsirolimus (CCI-779), Everolimus (RAD 001), Deforolimus (AP 23573) or TAFA 93.

7. The active substance combination according to claim 1, comprising
(A) Gemcitabine, and
(B) either
(B1) at least one Nodal/Activin-Inhibitor;
or
(B2) an active substance combination of
(B2a) at least one SHH inhibitor;
and
(B2b) at least one mTOR inhibitor.

8. The active substance combination (1) according to claim 1, comprising
(A) Gemcitabine, and
(B1) at least one Nodal/Activin-Inhibitor, wherein the Nodal/Activin-Inhibitor is SB431542, Coco-Protein, Nicalin-Protein or Nomo-Protein, Folistatin or Lefty.

9. The active substance combination (1) according to claim 8, wherein the combination is;
Gemcitabine and SB431542,
Gemcitabine and Coco-Protein,
Gemcitabine and Nicalin-Protein,
Gemcitabine and Nomo-Protein,
Gemcitabine and Folistatin, or
Gemcitabine and Lefty.

10. The active substance combination (1) according to claim 8, wherein the molecular ratio of nucleosid analog: (B1) Nodal/Activin-Inhibitor is about 1:0.0001-1.0.

11. The active substance combination (2) according to claim 1, comprising
(A) Gemcitabine,
(B2a) at least one SHH inhibitor, wherein the SHH inhibitor is Cyclopamine, Cyclopamine-KAAD, Jervine, CUR 61414, Forskolin, SANT-1, Arsenic Trioxide or CUR-0199691; and
(B2b) at least one mTOR inhibitor, wherein the mTOR inhibitor is Rapamycin, Temsirolimus (CCI-779), Everolimus (RAD 001), Deforolimus (AP 23573) or TAFA 93.

12. The active substance combination (1) according to claim 11, wherein the combination is;
Gemcitabine, Rapamycin and Cyclopamine,
Gemcitabine, Rapamycin and Cyclopamine-KAAD,
Gemcitabine, Rapamycin and Jervine,
Gemcitabine, Rapamycin and CUR 61414,
Gemcitabine, Rapamycin and Forskolin,
Gemcitabine, Rapamycin and SANT-1,
Gemcitabine, Rapamycin and Arsenic Trioxide,
Gemcitabine, Rapamycin and CUR-0199691,
Gemcitabine, Temsirolimus (CCI-779) and Cyclopamine,
Gemcitabine, Temsirolimus (CCI-779) and Cyclopamine-KAAD,
Gemcitabine, Temsirolimus (CCI-779) and Jervine,
Gemcitabine, Temsirolimus (CCI-779) and CUR 61414,
Gemcitabine, Temsirolimus (CCI-779) and Forskolin,
Gemcitabine, Temsirolimus (CCI-779) and SANT-1,
Gemcitabine, Temsirolimus (CCI-779) and Arsenic Trioxide,
Gemcitabine, Temsirolimus (CCI-779) and CUR-0199691,
Gemcitabine, Everolimus (RAD 001) and Cyclopamine,
Gemcitabine, Everolimus (RAD 001) and Cyclopamine-KAAD,
Gemcitabine, Everolimus (RAD 001) and Jervine,
Gemcitabine, Everolimus (RAD 001) and CUR 61414,
Gemcitabine, Everolimus (RAD 001) and Forskolin,
Gemcitabine, Everolimus (RAD 001) and SANT-1,
Gemcitabine, Everolimus (RAD 001) and Arsenic Trioxide,
Gemcitabine, Everolimus (RAD 001) and CUR-0199691,
Gemcitabine, Deforolimus (AP 23573) and Cyclopamine,
Gemcitabine, Deforolimus (AP 23573) and Cyclopamine-KAAD,
Gemcitabine, Deforolimus (AP 23573) and Jervine,
Gemcitabine, Deforolimus (AP 23573) and CUR 61414,
Gemcitabine, Deforolimus (AP 23573) and Forskolin,
Gemcitabine, Deforolimus (AP 23573) and SANT-1,
Gemcitabine, Deforolimus (AP 23573) and Arsenic Trioxide,
Gemcitabine, Deforolimus (AP 23573) and CUR-0199691,
Gemcitabine, TAFA 93 and Cyclopamine,
Gemcitabine, TAFA 93 and Cyclopamine-KAAD,
Gemcitabine, TAFA 93 and Jervine,
Gemcitabine, TAFA 93 and CUR 61414,
Gemcitabine, TAFA 93 and Forskolin,
Gemcitabine, TAFA 93 and SANT-1,
Gemcitabine, TAFA 93 and Arsenic Trioxide, or
Gemcitabine, TAFA 93 and CUR-0199691.

13. The active substance combination (2) according to claim 11, in which the molecular ratio of nucleoside analog: (B2a) SHH-Inhibitor: (B2b) mTor-Inhibitor is about 1:0.001-1.0:0.0001-0.01.

14. A pharmaceutical composition comprising an active substance combination according to claim 1 and optionally at least one or more physiologically acceptable auxiliary materials or additives.

15. A method for the treatment of cancer, comprising administering an active substance combination according to claim 1, for the treatment of epithelial tumours, pancreatic cancer, ovarian cancer, bladder cancer, colon cancer, breast cancer, leukemia, lung cancer, or brain tumour.

16. The method according to claim 15 wherein the treatment is epithelial cancer, pancreatic cancer, colon cancer, breast cancer, leukemia, or non small cell lung cancer (adeno carcinoma).

17. The active substance combination according to claim 2 wherein the pyrimidine analog is , including, gemcitabine, 5-Fluoruracil, Capecitabine, Cytarabine (Ara-C), and Floxuridine.

18. The active substance combination according to claim 2 wherein the purine analogs, is azathioprine, 6-mercaptopurine, 6-thioguanine, Fludarabine, or Pentostatin.

19. The active substance combination according to claim 2 wherein the purine antimetabolite is Fludarabine.

20. The active substance combination according to claim 2 wherein the Anthracycline is Daunorubicin, Doxorubicin (Adriamycin), Epirubicin, and Idarubicin.

21. The active substance combination according to claim 2 wherein the folate analog is methothrexate.

22. The active substance combination according to claim 2 wherein the Ribonucleotide reductase inhibitor is hydroxyurea.

23. The active substance combination according to claim 7, wherein the Nodal/Activin-Inhibitor is SB431542, Coco-Protein, Nicalin-Protein, Nomo-Protein, Folistatin or Lefty

24. The active substance combination according to claim 23, wherein the Nodal/Activin-Inhibitor is SB431542.

25. The active substance combination according to claim 7, wherein the SHH inhibitor is Cyclopamine, Cyclopamine-KAAD, Jervine, CUR 61414, Forskolin, SANT-1, Arsenic Trioxide or CUR-0199691.

26. The active substance combination according to claim 25, wherein the SHH inhibitor is Cyclopamine.

27. The active substance combination according to claim 7, wherein the mTOR inhibitor is Rapamycin, Temsirolimus (CCI-779), Everolimus (RAD 001), Deforolimus (AP 23573) or TAFA 93.

28. The active substance combination according to claim 27, wherein the mTOR inhibitor is Rapamycin.
